(19) United States
(12) Patent Application Publication
(10) Pub. No.: US 2025/0145558 A1
(43) Pub. Date: May 8, 2025

(54) SERINOL DERIVATIVES FOR CAPTURING ACID GASES AND METHODS OF MAKING AND USE THEREOF

(71) Applicant: The Board of Trustees of The University of Alabama, Tuscaloosa, AL (US)

(72) Inventor: Jason E. Bara, Tuscaloosa, AL (US)

(21) Appl. No.: 18/814,951

(22) Filed: Aug. 26, 2024

Related U.S. Application Data
(60) Provisional application No. 63/595,369, filed on Nov. 2, 2023.

(51) Int. Cl. C07C 211/08; B01D 53/14

(52) U.S. Cl. CPC C07C 211/08; B01D 53/1456; B01D 2252/20415; B01D 2257/504

(57) ABSTRACT

Disclosed herein are serinol derivatives for capturing acid gases and methods of making and use thereof.

### CROSS-REFERENCE TO RELATED APPLICATIONS

This application claims the benefit of priority to U.S. Provisional Application No. 63/595,369 filed Nov. 2, 2023, which is hereby incorporated herein by reference in its entirety.

### STATEMENT OF GOVERNMENT SUPPORT

This invention was made with government support under Grant No. 2132133 awarded by the National Science Foundation. The government has certain rights in the invention.

### BACKGROUND

Improved compounds and methods for capturing acid gases, such as CO₂, are needed. The compositions and methods discussed herein address these and other needs.

### SUMMARY

In accordance with the purposes of the disclosed compositions and methods as embodied and broadly described herein, the disclosed subject matter relates to serinol derivatives for capturing acid gases and methods of making and use thereof.

For example, disclosed herein are compounds for capturing an acid gas from a fluid stream, wherein the compounds are defined by Formula I:

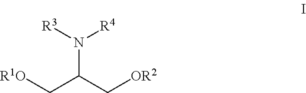

wherein R¹ and R² are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy; and R³ and R⁴ are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy, or wherein, as valence permits, R³ and R⁴, together with the atoms to which they are attached, form a 3-10 membered substituted or unsubstituted cyclic moiety, optionally including 1 to 3 additional heteroatoms; with the proviso that at least one of R¹-R⁴ is not H.

Also disclosed herein are solvent systems for capturing an acid gas from a fluid stream, wherein the solvent system comprises any of the compounds disclosed herein.

Also disclosed herein are methods of use of any of the compounds or any of the solvent systems disclosed herein for capturing an acid gas from a fluid steam, the methods comprising contacting the compound or solvent system with the fluid stream, wherein the fluid stream comprises the acid gas, thereby capturing the acid gas from the fluid stream.

Additional advantages of the disclosed compositions and methods will be set forth in part in the description which follows, and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. Schematic diagram of CO₂ capture process.

FIG. 2. Glycerol and associated “activated” forms (top). Transformation to green chemical platforms (bottom) through which the pace of decarbonization CO₂ capture can be enhanced.

FIG. 3. General relationship between CO₂ absorption capacity and partial pressure for chemical (red) and physical (blue) solvents.

### DETAILED DESCRIPTION

The compositions and methods described herein may be understood more readily by reference to the following detailed description of specific aspects of the disclosed subject matter and the Examples included therein.

Before the present compositions and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

Values can be expressed herein as an “average” value. “Average” generally refers to the statistical mean value.

By “substantially” is meant within 5%, e.g., within 4%, 3%, 2%, or 1%.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, “molecular weight” refers to number average molecular weight as measured by ¹H NMR spectroscopy, unless indicated otherwise.

As used herein, a “fluid” includes a liquid, a gas, a supercritical fluid, or a combination thereof.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure-CH═CH₂; 1-propenyl refers to a group with the structure —CH—CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some examples, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O) H. Throughout this specification “C(O)” is a shorthand notation for C═O.

The term “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

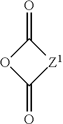

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O.

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

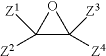

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein comprises the formula —S—.

The term “thiol” as used herein is represented by the formula —SH.

“R¹,” “R²,” “R³,” “R^{a}”,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amino group, an alkyl group, a halide, and the like. Depending upon the groups that are selected, a first group can be incorporated within a second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As used herein, Me refers to a methyl group; OMe refers to a methoxy group; and i-Pr refers to an isopropyl group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Compounds

Disclosed herein are compounds and methods of making and use thereof. For example, disclosed herein are compounds for capturing an acid gas from a fluid stream.

The term “acid gas” as used herein refers to chemical compounds that are capable of vaporizing to a significant amount or that exist as a gas at ambient conditions with significant quantities of one or more gases that can be considered a Lewis Acid. A compound is a Lewis Acid if the compound can act as an electron pair acceptor. The “acid gases” described herein are found in fluid streams, such as natural gas feeds. Examples of acid gases include CO₂, CO, COS, H₂S, SO₂, NO, N₂O, mercaptans, Cl₂, volatile organic compounds, and mixtures of these.

By “capture” or other forms of the word, such as “capturing” or “captured,” is meant lowering of a characteristic (e.g., lowering the amount of an acid gas in a fluid stream). For example, “captures CO₂” means an amount of CO₂ is removed from a fluid stream relative. As used herein, capture can include complete removal. In the disclosed methods, capture can refer to a 10 mol %, 20 mol %, 30 mol %, 40 mol %, 50 mol %, 60 mol %, 70 mol %, 80 mol %, 90 mol %, or 100 mol % decrease as compared to the standard or a control. It is understood that the terms “sequester,” “remove,” and “separation” are used synonymously with the term “capture.”

For example, disclosed herein are compounds for capturing an acid gas from a fluid stream, wherein the compounds are defined by Formula I:

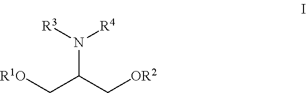

wherein
R¹ and R² are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy; and
R³ and R⁴ are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy, or wherein, as valence permits, R³ and R⁴, together with the atoms to which they are attached, form a 3-10 membered substituted or unsubstituted cyclic moiety, optionally including 1 to 3 additional heteroatoms;
with the proviso that at least one of R¹-R⁴ is not H (e.g., the compound is not serinol).

In some examples of Formula I, R³ and R⁴ are the same. In some examples of Formula I, R³ and R⁴ are different.

In some examples of Formula I, R³ and R⁴ are each independently H, substituted or unsubstituted C₁₋₂₀ alkyl, or substituted or unsubstituted C₁-C₂₀ alkoxy, or wherein, as valence permits, R³ and R⁴, together with the atoms to which they are attached, form a 3-10 membered substituted or unsubstituted cyclic moiety, optionally including 1 to 3 additional heteroatoms.

In some examples of Formula I, R³ and R⁴ are each independently H, substituted or unsubstituted C₁₋₆ alkyl, or substituted or unsubstituted C₁-C₆ alkoxy, or wherein, as valence permits, R³ and R⁴, together with the atoms to which they are attached, form a 6 membered substituted or unsubstituted cyclic moiety, optionally including 1 additional heteroatom.

In some examples of Formula I, R³ and R⁴ are each independently H, methyl, ethyl, isopropyl, or —CH₂CH₂OCH₃, or R³ and R⁴, together with the atoms to which they are attached, form a piperazine.

In some examples of Formula I, R⁴ is hydrogen.

In some examples of Formula I, R³ is methyl, ethyl, isopropyl, or —CH₂CH₂OCH₃.

In some examples of Formula I, R⁴ is hydrogen and R³ is not hydrogen.

In some examples of Formula I, R⁴ is hydrogen and R³ is methyl, ethyl, isopropyl, or —CH₂CH₂OCH₃.

In some examples of Formula I, R³ and R⁴ are each hydrogen.

In some examples of Formula I, neither R³ nor R⁴ is hydrogen.

In some examples of Formula I, R³ and R⁴ are each independently substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy, or wherein, as valence permits, R³ and R⁴, together with the atoms to which they are attached, form a 3-10 membered substituted or unsubstituted cyclic moiety, optionally including 1 to 3 additional heteroatoms.

In some examples of Formula I, R³ and R⁴ are each independently substituted or unsubstituted C₁₋₂₀ alkyl, or substituted or unsubstituted C₁-C₂₀ alkoxy, or wherein, as valence permits, R³ and R⁴, together with the atoms to which they are attached, form a 3-10 membered substituted or unsubstituted cyclic moiety, optionally including 1 to 3 additional heteroatoms.

In some examples of Formula I, wherein R³ and R⁴ are each independently substituted or unsubstituted C₁₋₆ alkyl, or substituted or unsubstituted C₁-C₆ alkoxy, or wherein, as valence permits, R³ and R⁴, together with the atoms to which they are attached, form a 6 membered substituted or unsubstituted cyclic moiety, optionally including 1 additional heteroatom.

In some examples of Formula I, R³ and R⁴ are each independently methyl, ethyl, isopropyl, or —CH₂CH₂OCH₃, or R³ and R⁴, together with the atoms to which they are attached, form a piperazine.

In some examples of Formula I, R³ and R⁴ are each independently methyl, ethyl, isopropyl, or —CH₂CH₂OCH₃.

In some examples of Formula I, R³ and R⁴ together with the atoms to which they are attached, form a piperazine.

In some examples of Formula I, R¹ and R² are the same. In some examples of Formula I, R¹ and R² are different.

In some examples of Formula I, R¹ and R² are each independently substituted or unsubstituted C₁₋₂₀ alkyl, or substituted or unsubstituted C₁-C₂₀ alkoxy.

In some examples of Formula I, R¹ and R² are each independently substituted or unsubstituted C₁₋₆ alkyl, or substituted or unsubstituted C₁-C₆ alkoxy.

In some examples of Formula I, R¹ and R² are each independently C₁₋₆ alkyl optionally substituted with one or more substituents selected from the group consisting of ether, C₁-C₅ alkyl, halogen, amine, and C₁-C₅ alkoxy.

In some examples of Formula I, R¹ and R² are each independently C₁₋₆ alkyl optionally substituted with halogen (e.g., C₁-C₆ fluoroalkyl, such as trifluoromethyl terminated alkyl). In some examples of Formula I, R¹ and R² are each independently methyl, ethyl, isopropyl, —CH₂CF₃, —CH₂CH₂OCH₃.

In some examples of Formula I, R¹ and R² are each independently substituted or unsubstituted C₁₋₂₀ alkyl, or substituted or unsubstituted C₁-C₂₀ alkoxy; and R³ and R⁴ are each independently H, substituted or unsubstituted C₁₋₂₀ alkyl, or substituted or unsubstituted C₁-C₂₀ alkoxy, or wherein, as valence permits, R³ and R⁴, together with the atoms to which they are attached, form a 3-10 membered substituted or unsubstituted cyclic moiety, optionally including 1 to 3 additional heteroatoms.

In some examples of Formula I, R¹ and R² are each independently substituted or unsubstituted C₁₋₆ alkyl, or substituted or unsubstituted C₁-C₆ alkoxy; and R³ and R⁴ are each independently H, substituted or unsubstituted C₁₋₆ alkyl, or substituted or unsubstituted C₁-C₆ alkoxy, or wherein, as valence permits, R³ and R⁴, together with the atoms to which they are attached, form a 6 membered substituted or unsubstituted cyclic moiety, optionally including 1 additional heteroatom.

In some examples of Formula I, R¹ and R² are each independently methyl, ethyl, isopropyl, —CH₂CF₃, —CH₂CH₂OCH₃; and R³ and R⁴ are each independently H, methyl, ethyl, isopropyl, or —CH₂CH₂OCH₃, or R³ and R⁴, together with the atoms to which they are attached, form a piperazine.

In some examples of Formula I, the compound is selected from the group consisting of:

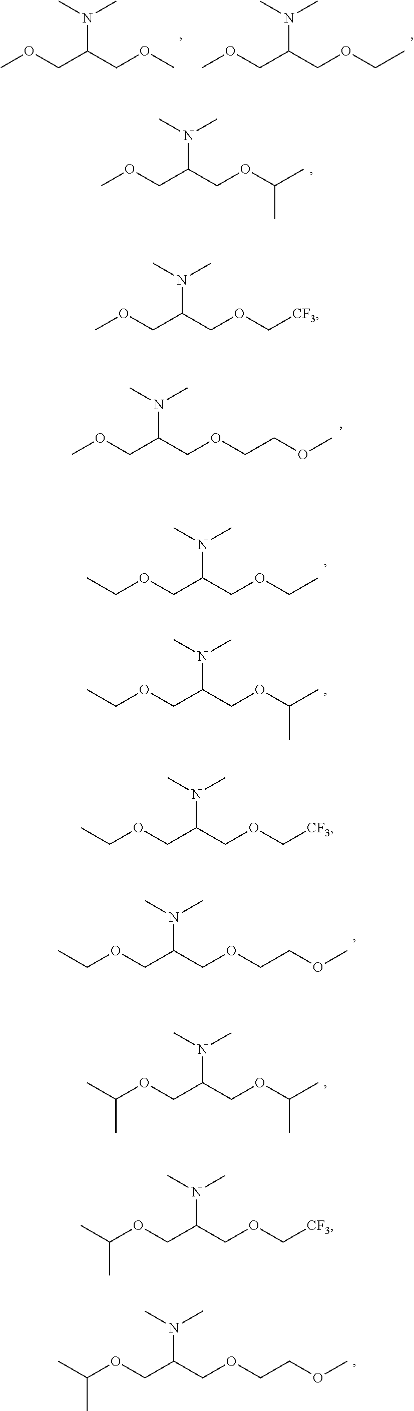

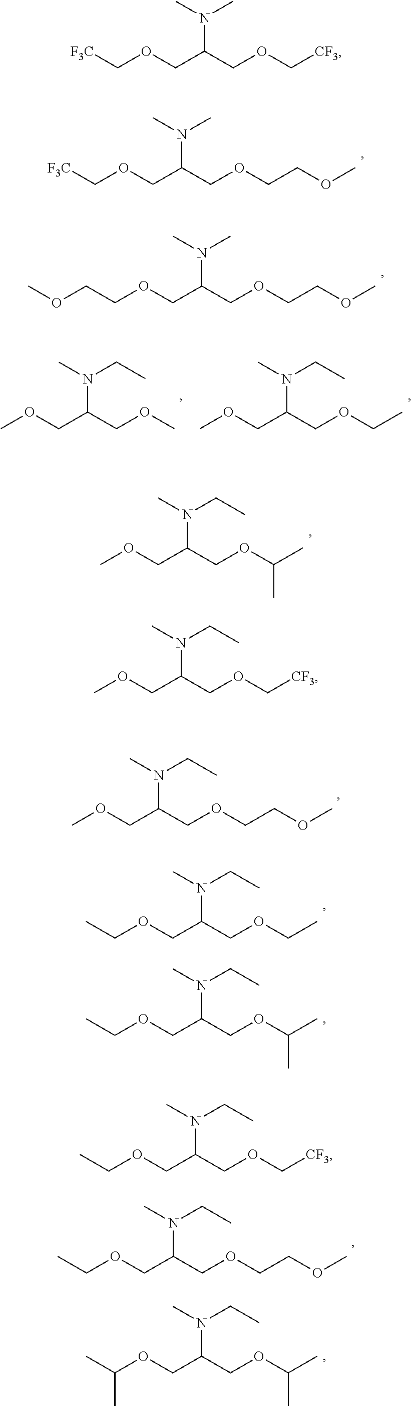

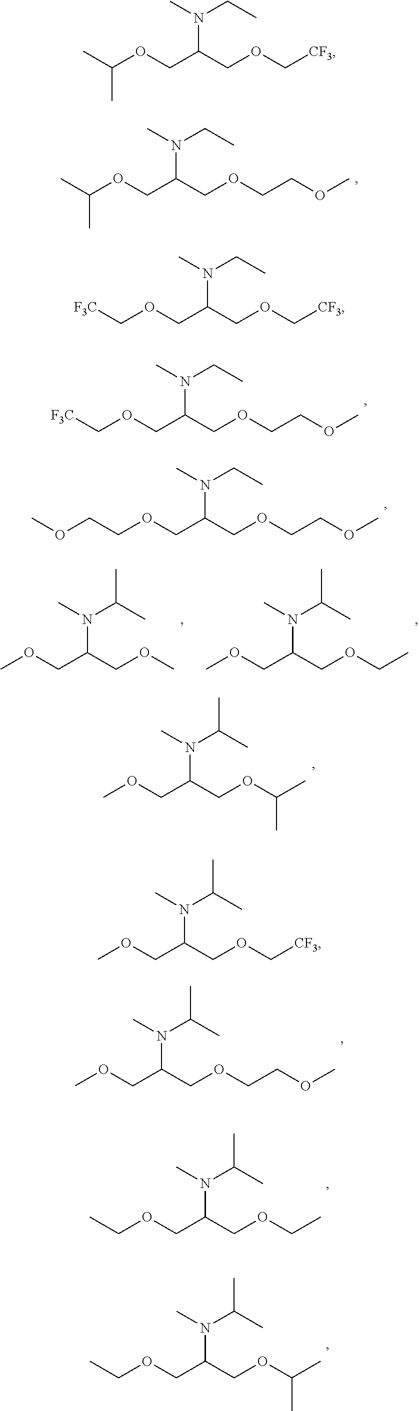

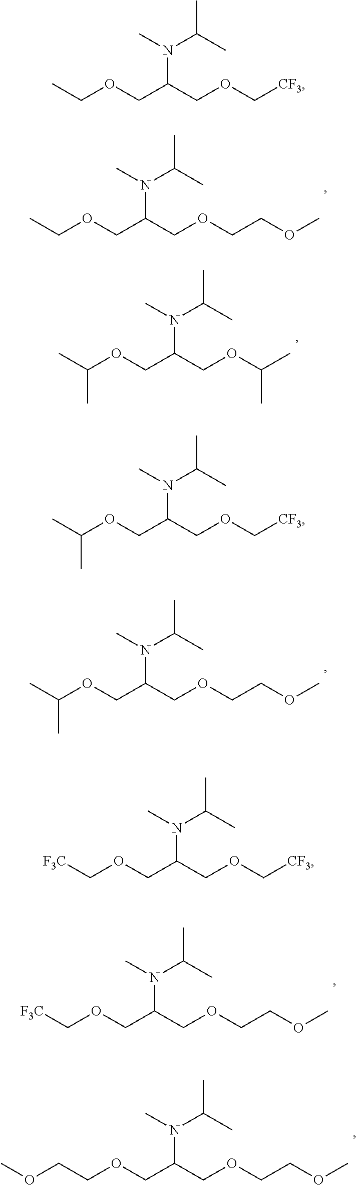

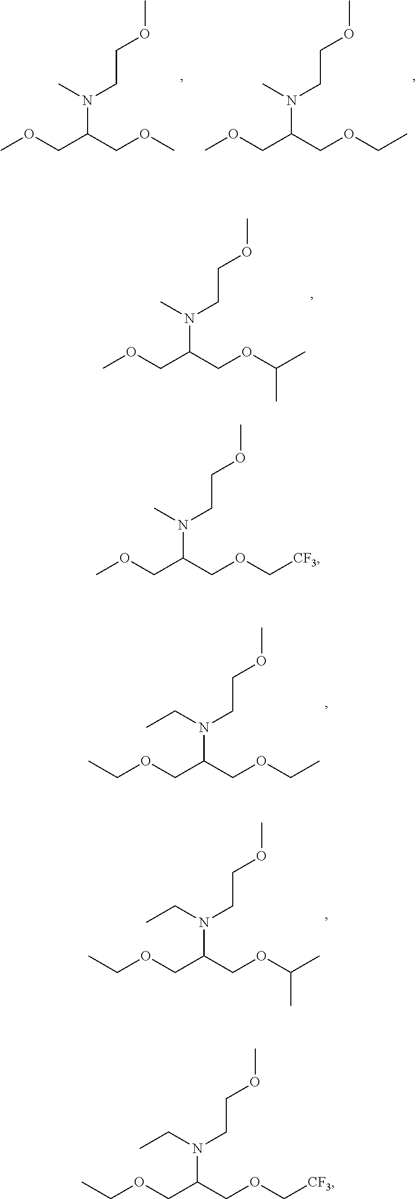

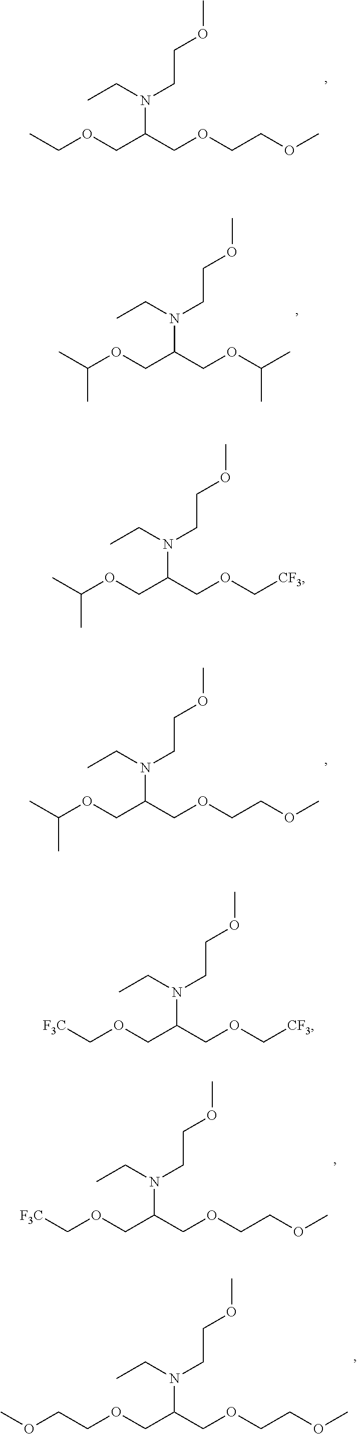

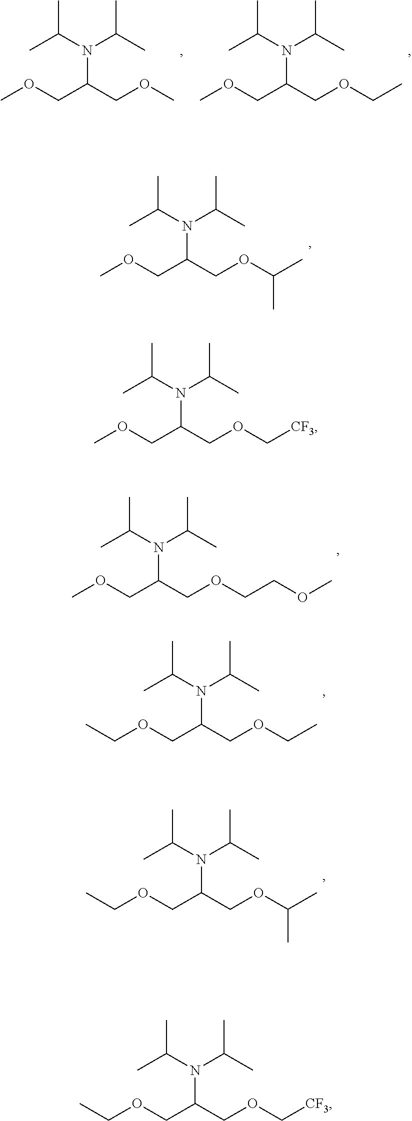

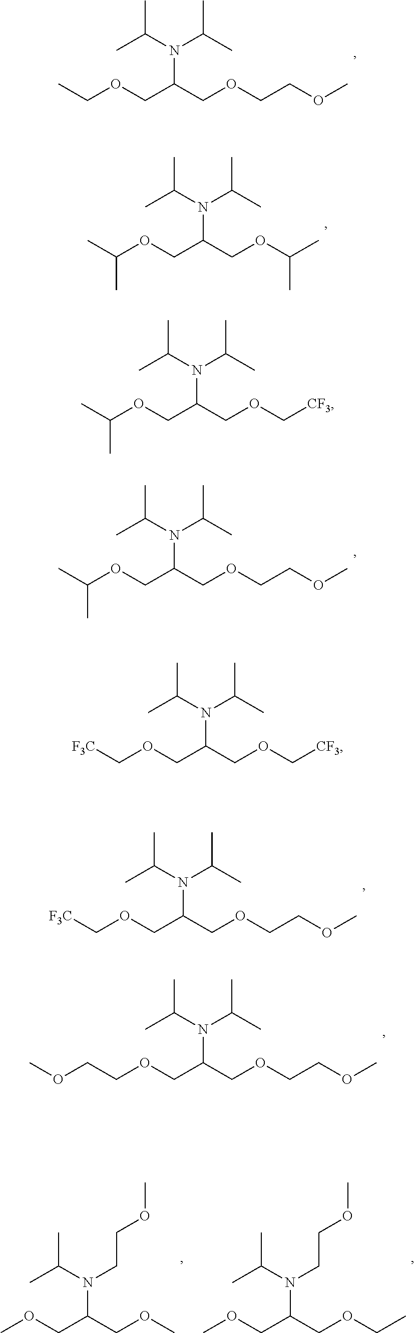

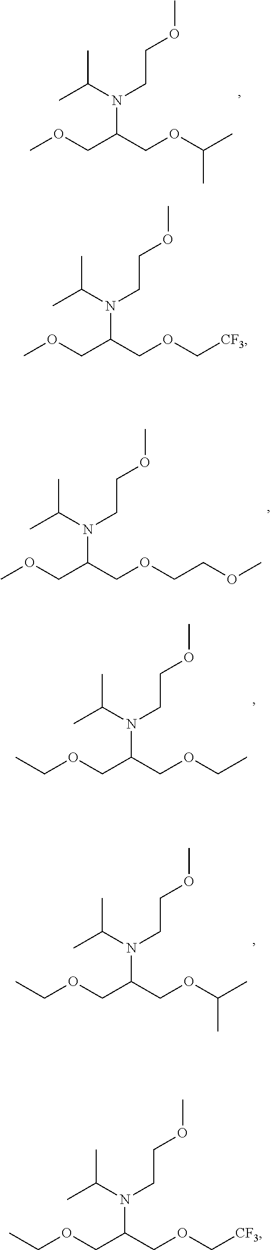

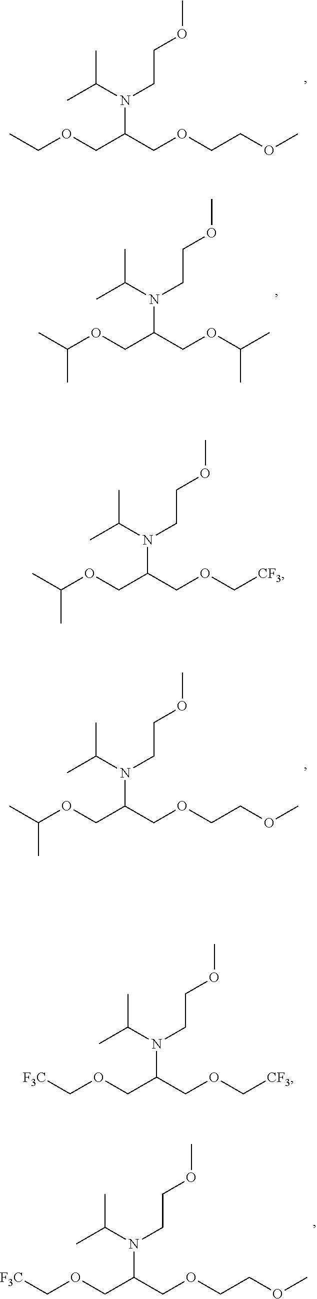

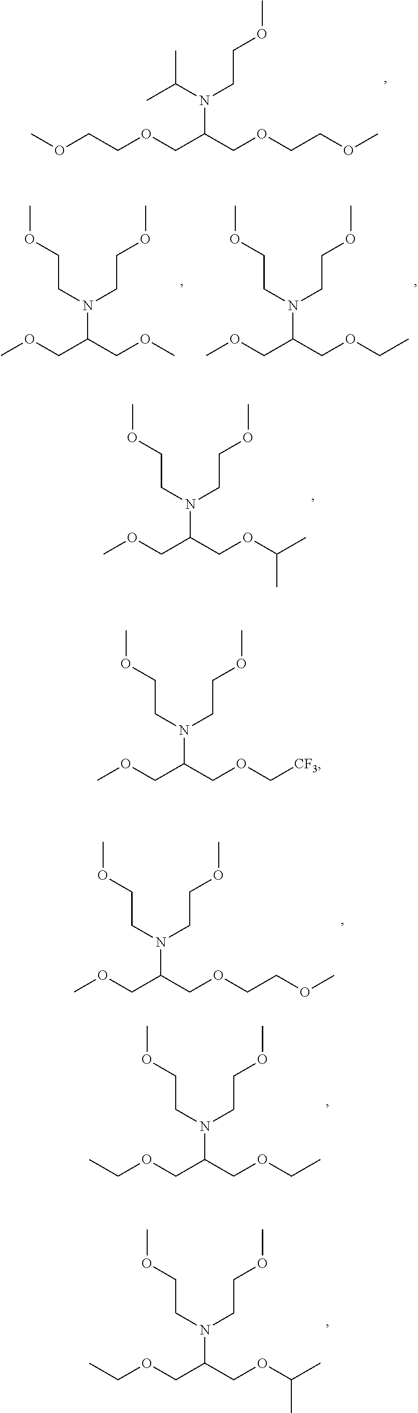

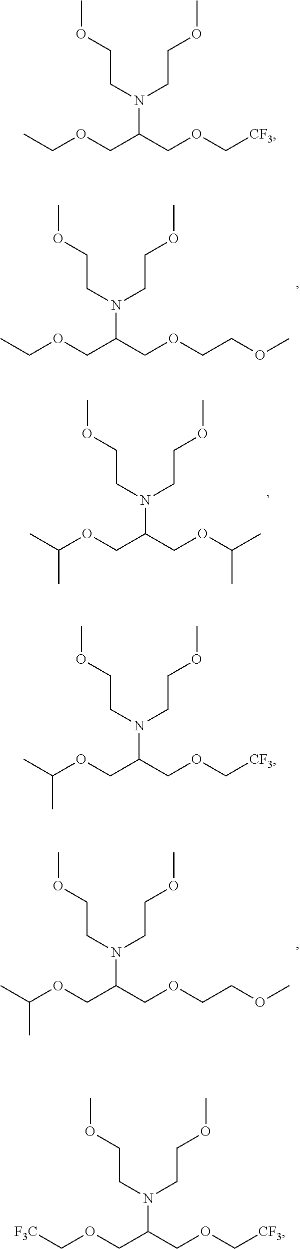

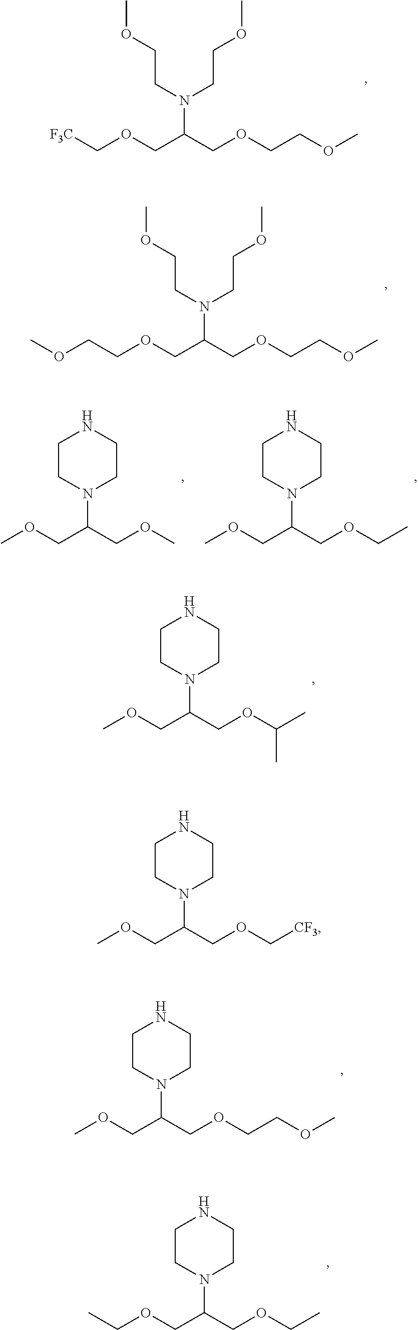

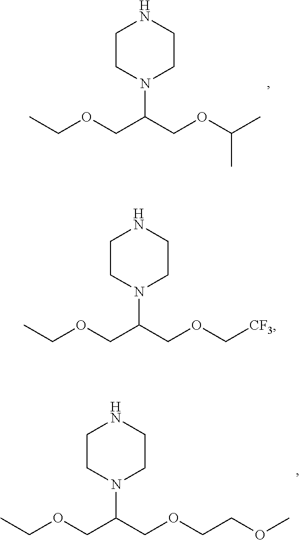

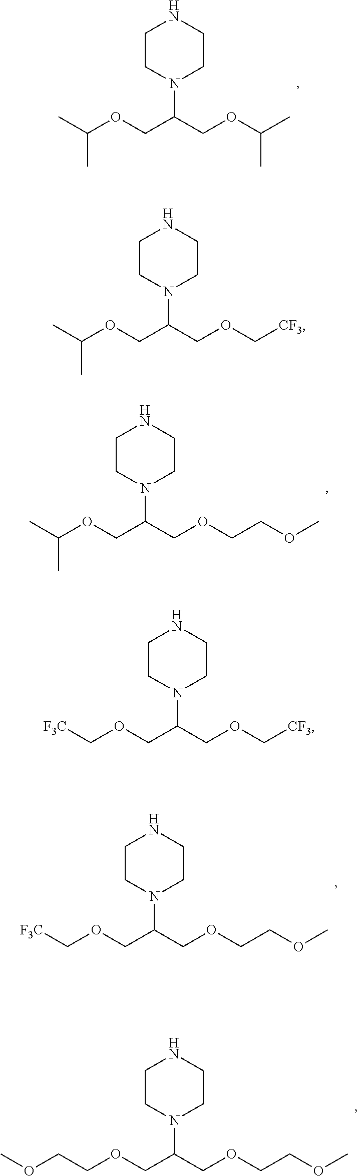

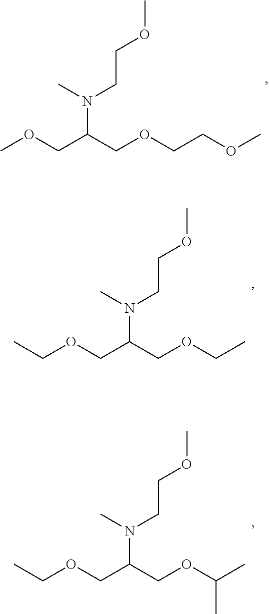

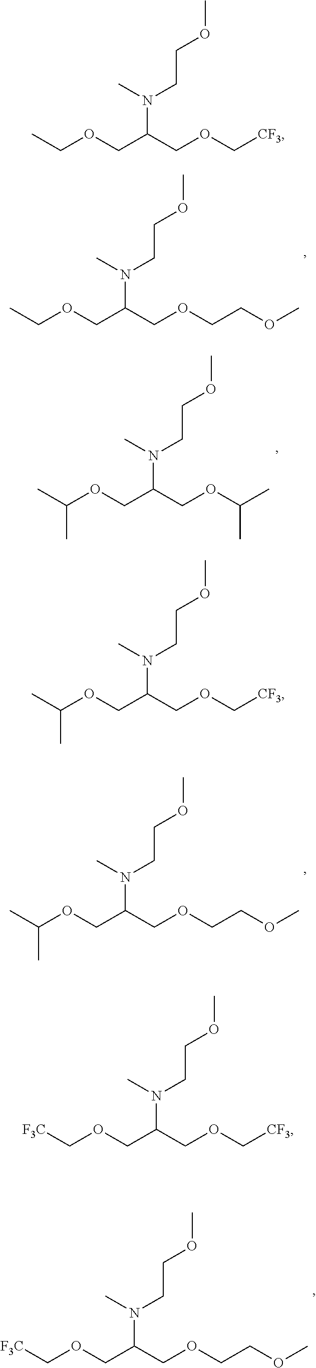

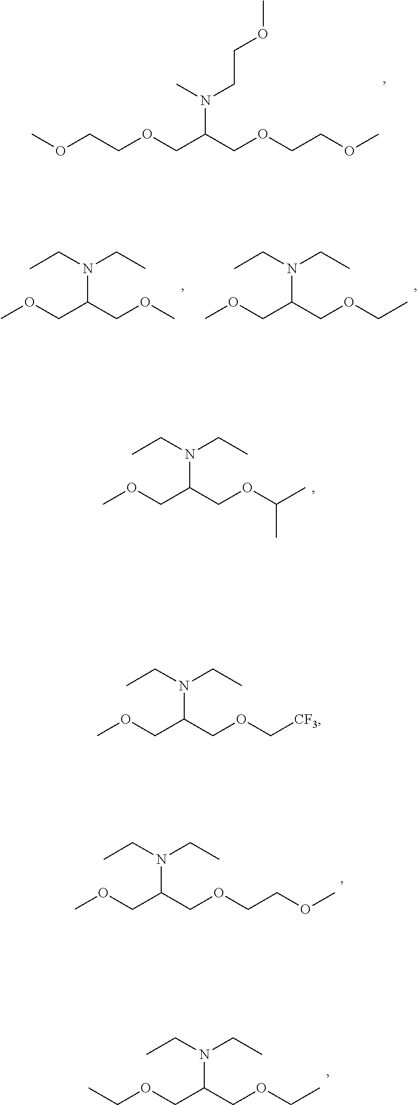

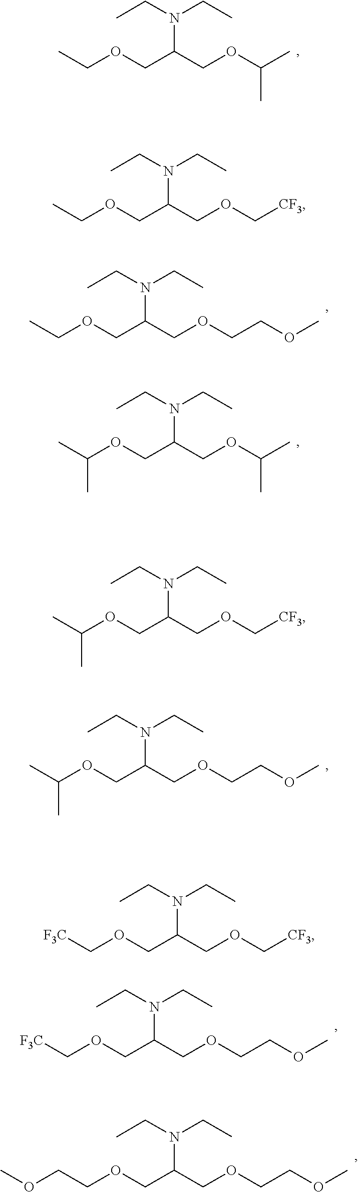

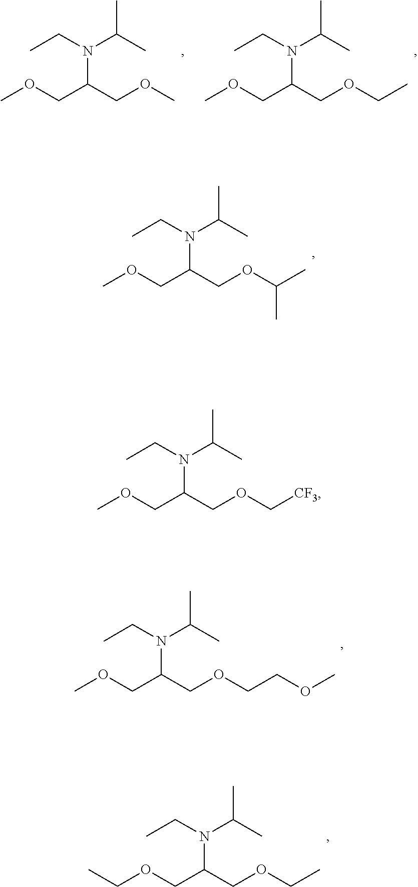

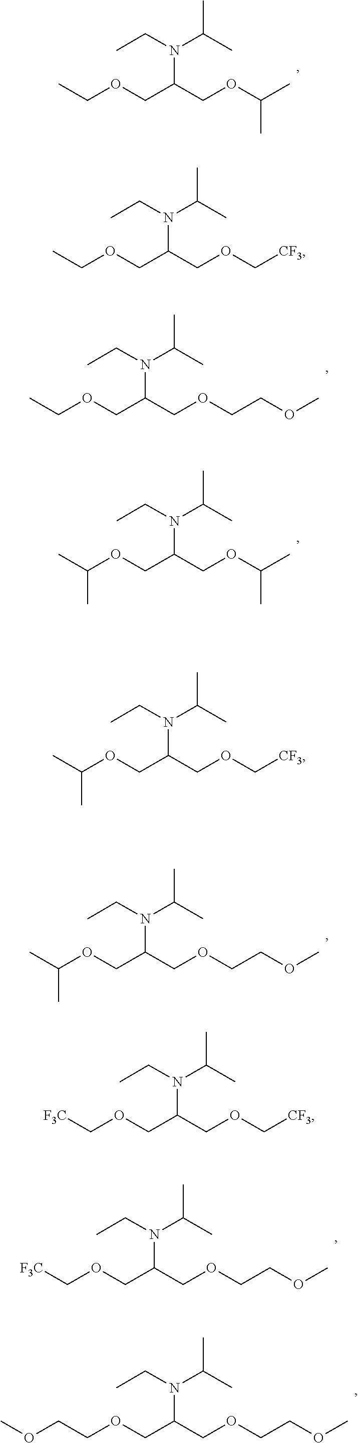

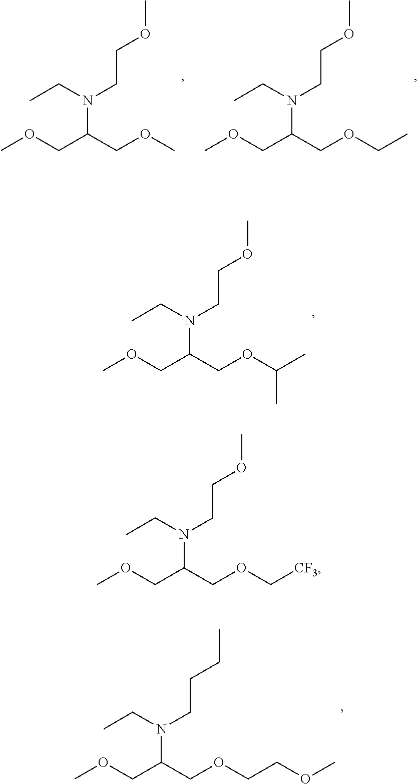

and combinations thereof.

In some examples of Formula I, the compound is selected from the group consisting of:

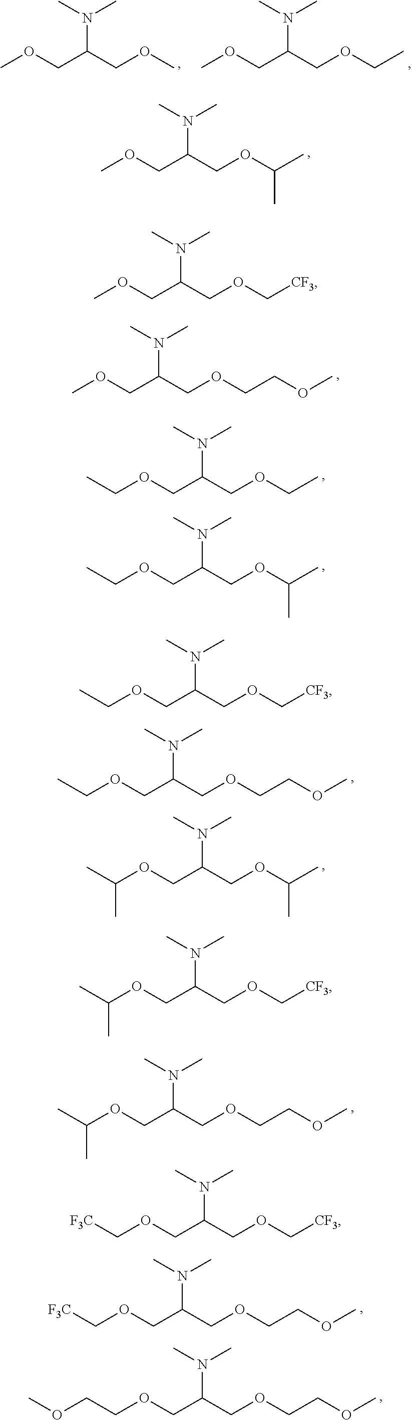

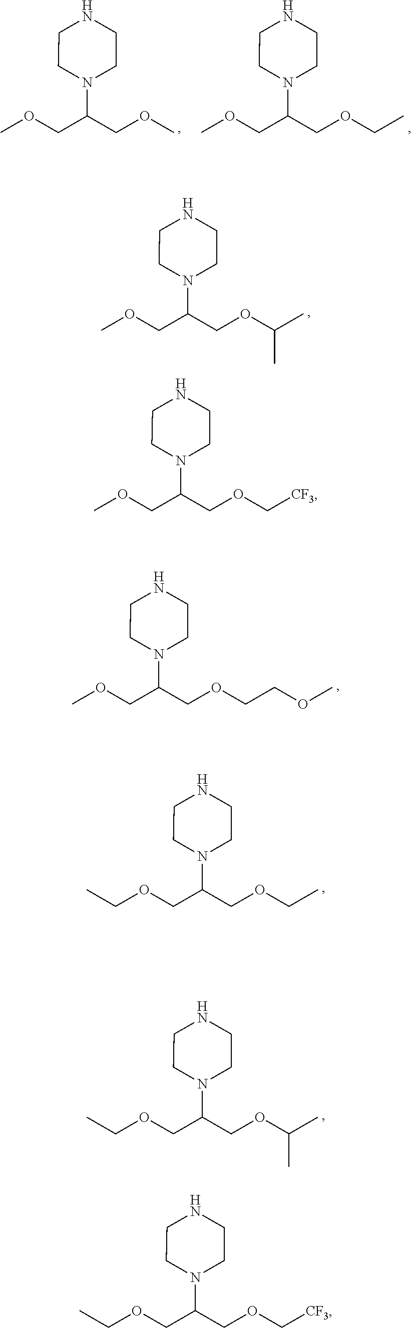

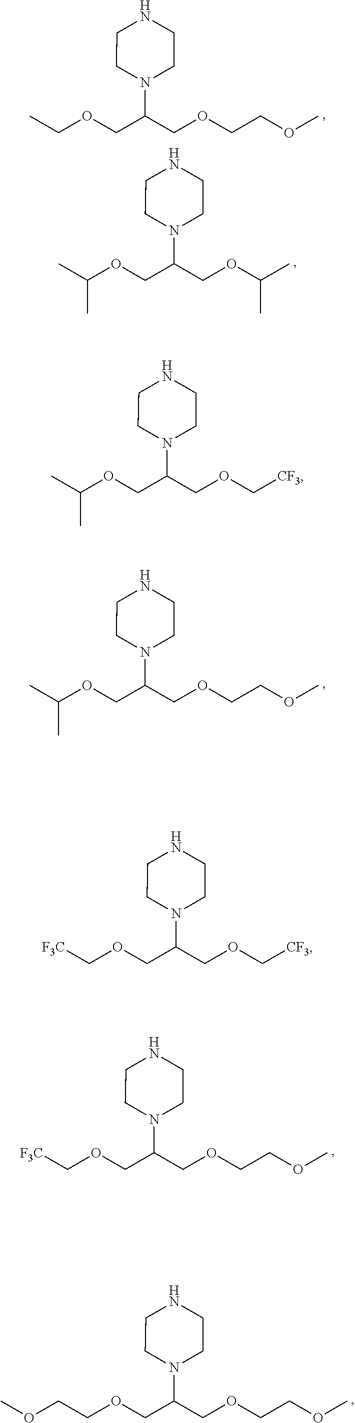

and combinations thereof.

In some examples of Formula I, the compound is selected from the group consisting of:

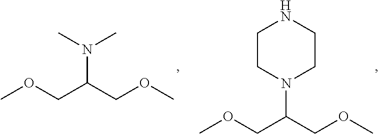

and combinations thereof.

In some examples, the compound is defined by Formula II:

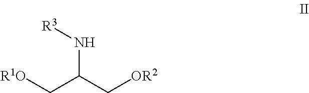

wherein
R¹ and R² are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy; and
R³ is H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy, or wherein, as valence permits, R³ and R⁴, together with the atoms to which they are attached, form a 3-10 membered substituted or unsubstituted cyclic moiety, optionally including 1 to 3 additional heteroatoms;
with the proviso that at least one of R¹-R³ is not H.

In some examples of Formula II, R³ is H. In some examples of Formula II, R³ is not H.

In some examples of Formula II, R³ is substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy.

In some examples of Formula II, R³ is H, substituted or unsubstituted C₁₋₂₀ alkyl, or substituted or unsubstituted C₁-C₂₀ alkoxy.

In some examples of Formula II, R³ is substituted or unsubstituted C₁₋₂₀ alkyl, or substituted or unsubstituted C₁-C₂₀ alkoxy.

In some examples of Formula II, R³ is H, substituted or unsubstituted C₁₋₆ alkyl, or substituted or unsubstituted C₁-C₆ alkoxy.

In some examples of Formula II, R³ is substituted or unsubstituted C₁₋₆ alkyl, or substituted or unsubstituted C₁-C₆ alkoxy.

In some examples of Formula II, R³ is H, methyl, ethyl, isopropyl, or —CH₂CH₂OCH₃.

In some examples of Formula II, R³ is methyl, ethyl, isopropyl, or —CH₂CH₂OCH₃.

In some examples of Formula II, R¹ and R² are the same. In some examples of Formula II, R¹ and R² are different.

In some examples of Formula II, R¹ and R² are each independently substituted or unsubstituted C₁₋₂₀ alkyl, or substituted or unsubstituted C₁-C₂₀ alkoxy.

In some examples of Formula II, R¹ and R² are each independently substituted or unsubstituted C₁₋₆ alkyl, or substituted or unsubstituted C₁-C₆ alkoxy.

In some examples of Formula II, R¹ and R² are each independently C₁₋₆ alkyl optionally substituted with one or more substituents selected from the group consisting of ether, C₁-C₅ alkyl, halogen, amine, and C₁-C₈ alkoxy.

In some examples of Formula II, R¹ and R² are each independently C₁₋₆ alkyl optionally substituted with halogen (e.g., C₁-C₆ fluoroalkyl, such as trifluoromethyl terminated alkyl).

In some examples of Formula II, R¹ and R² are each independently methyl, ethyl, isopropyl, —CH₂CF₃, —CH₂CH₂OCH₃.

In some examples of Formula II, R¹ and R² are each independently substituted or unsubstituted C₁₋₂₀ alkyl, or substituted or unsubstituted C₁-C₂₀ alkoxy; and R³ is H, substituted or unsubstituted C₁₋₂₀ alkyl, or substituted or unsubstituted C₁-C₂₀ alkoxy.

In some examples of Formula II, R¹ and R² are each independently substituted or unsubstituted C₁₋₆ alkyl, or substituted or unsubstituted C₁-C₆ alkoxy; and R³ is H, substituted or unsubstituted C₁₋₆ alkyl, or substituted or unsubstituted C₁-C₆ alkoxy.

In some examples of Formula II, R¹ and R² are each independently methyl, ethyl, isopropyl, —CH₂CF₃, —CH₂CH₂OCH₃; and R³ is H, methyl, ethyl, isopropyl, or —CH₂CH₂OCH₃.

In some examples of Formula I or Formula II, the compound is selected from the group consisting of:

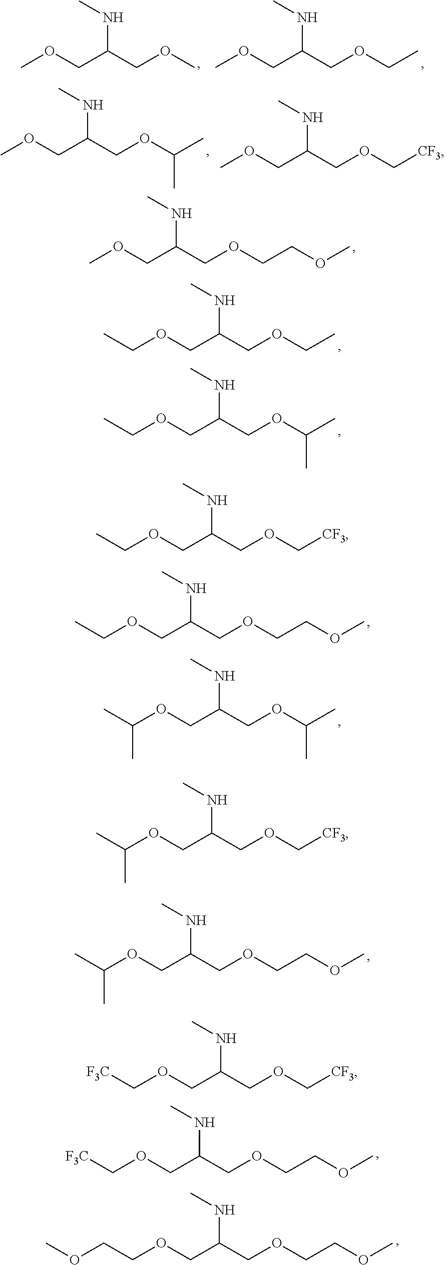

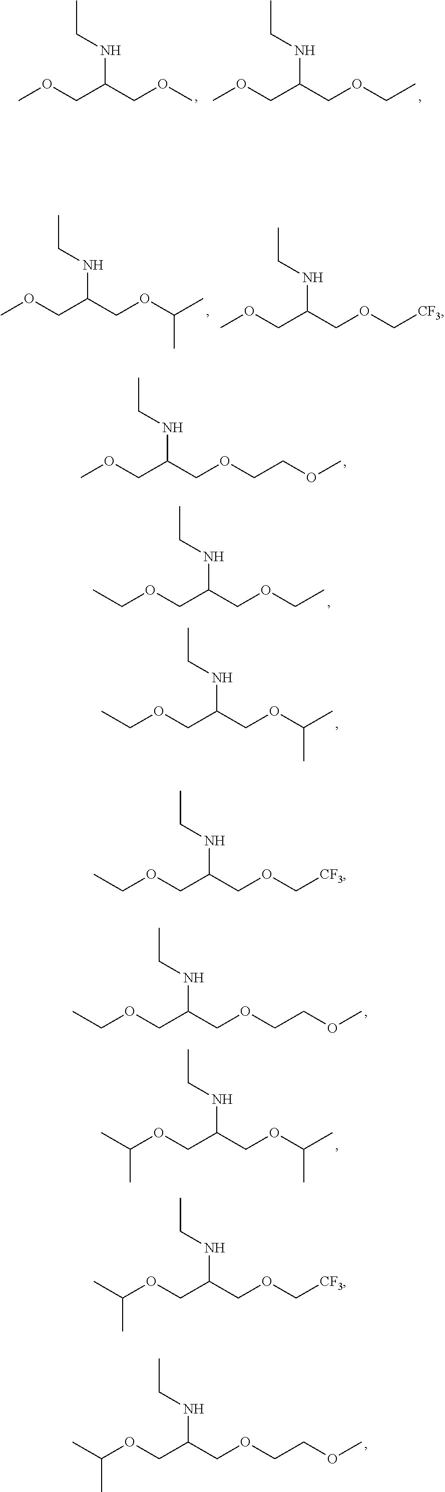

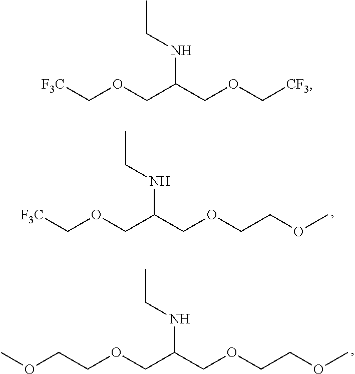

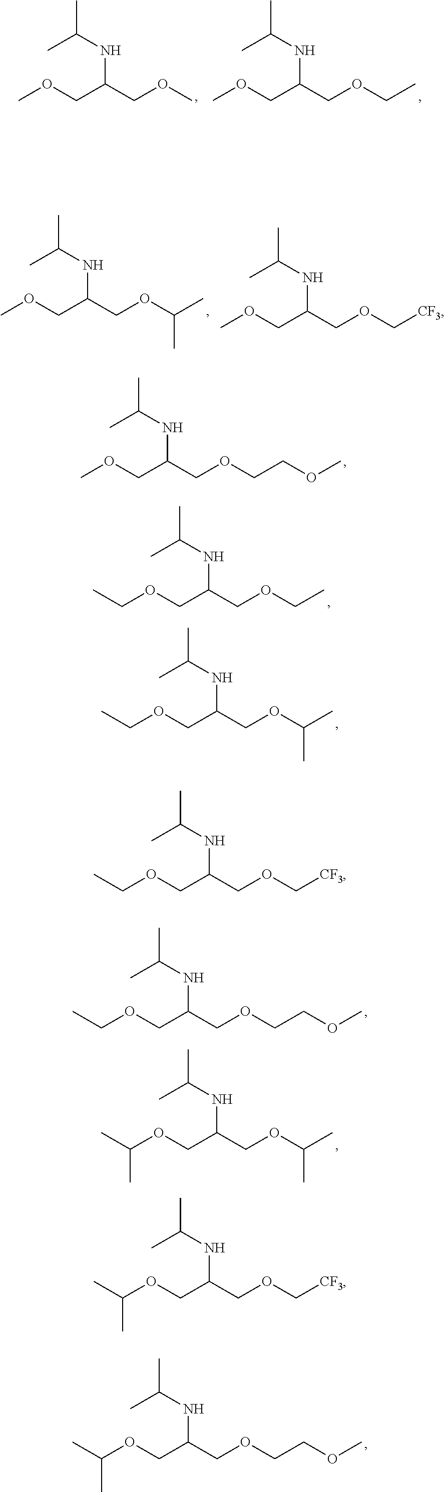

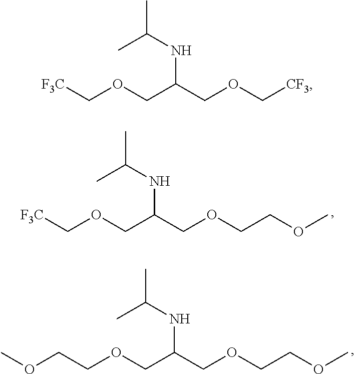

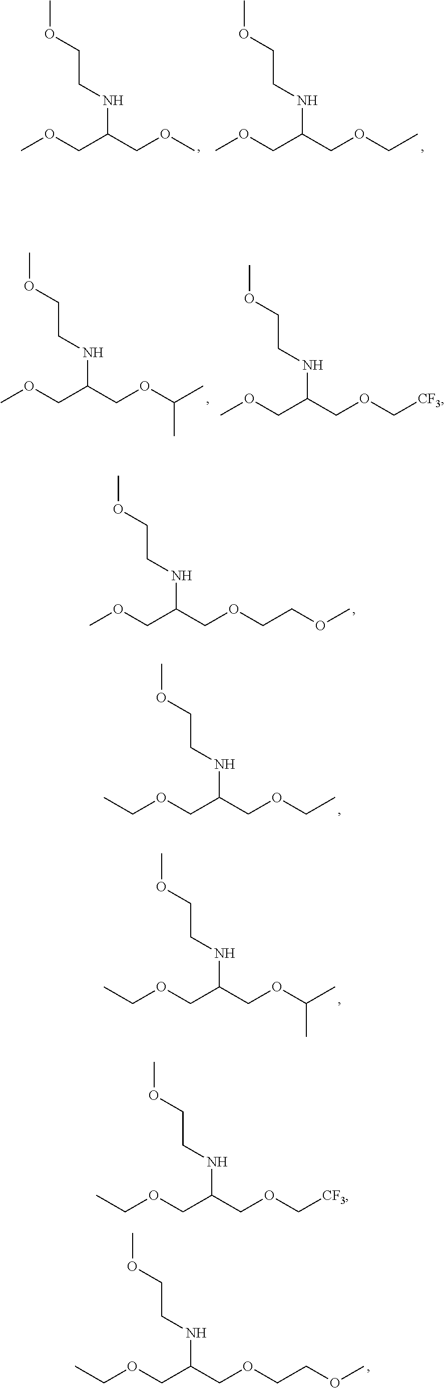

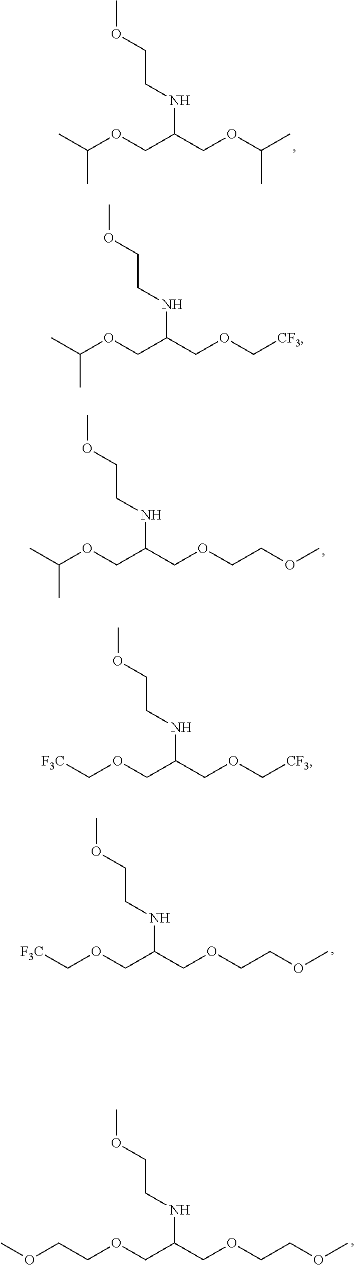

and combinations thereof.

In some examples of Formula I or Formula II, the compound is selected from the group consisting of:

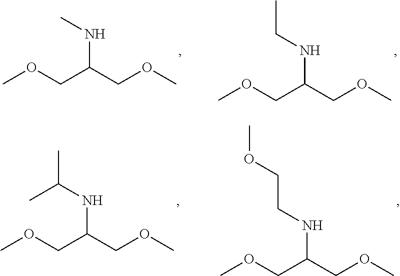

and combinations thereof.

In some examples, the compound is defined by Formula III:

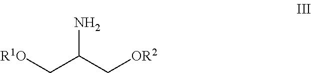

wherein
R¹ and R² are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy;
with the proviso that at least one of R¹-R² is not H.

In some examples of Formula III, R¹ and R² are the same. In some examples of Formula III, R¹ and R² are different.

In some examples, neither R¹ nor R² is hydrogen.

In some examples of Formula III, R¹ and R² are each independently substituted or unsubstituted C₁₋₂₀ alkyl, or substituted or unsubstituted C₁-C₂₀ alkoxy.

In some examples of Formula III, R¹ and R² are each independently substituted or unsubstituted C₁₋₆ alkyl, or substituted or unsubstituted C₁-C₆ alkoxy.

In some examples of Formula III, R¹ and R² are each independently C₁₋₆ alkyl optionally substituted with one or more substituents selected from the group consisting of ether, C₁-C₅ alkyl, halogen, amine, and C₁-C₈ alkoxy.

In some examples of Formula III, R¹ and R² are each independently C₁₋₆ alkyl optionally substituted with halogen (e.g., C₁-C₆ fluoroalkyl, such as trifluoromethyl terminated alkyl).

In some examples of Formula III, R¹ and R² are each independently methyl, ethyl, isopropyl, —CH₂CF₃, —CH₂CH₂OCH₃.

In some examples of Formula I or Formula III, the compound is selected from the group consisting of:

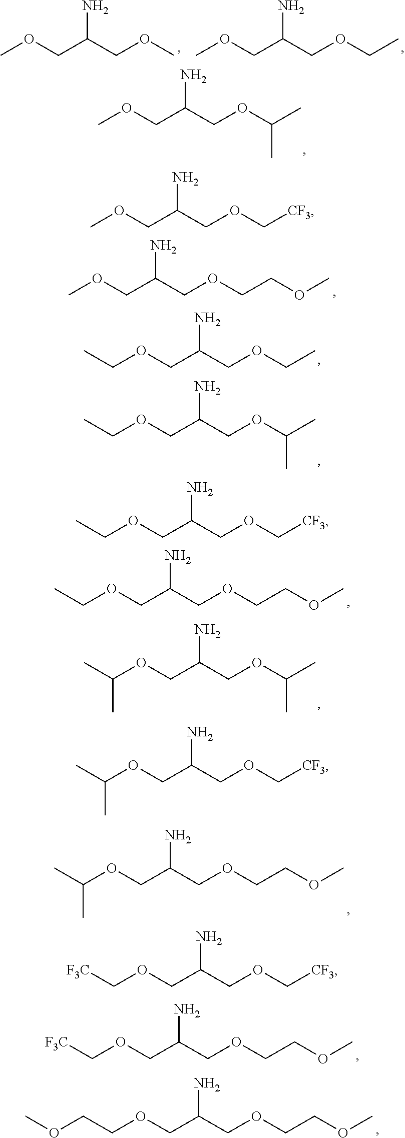

and combinations thereof.

In some examples of Formula I or Formula III, the compound is selected from the group consisting of:

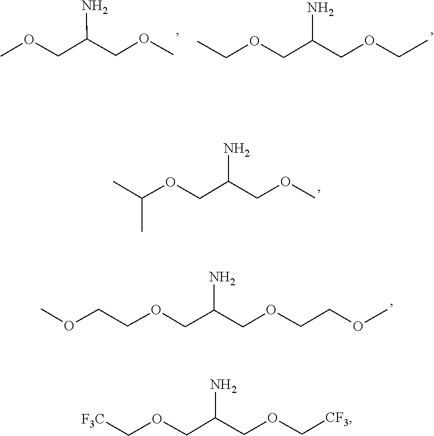

and combinations thereof.

In some examples, the compound is selected from the group consisting of:

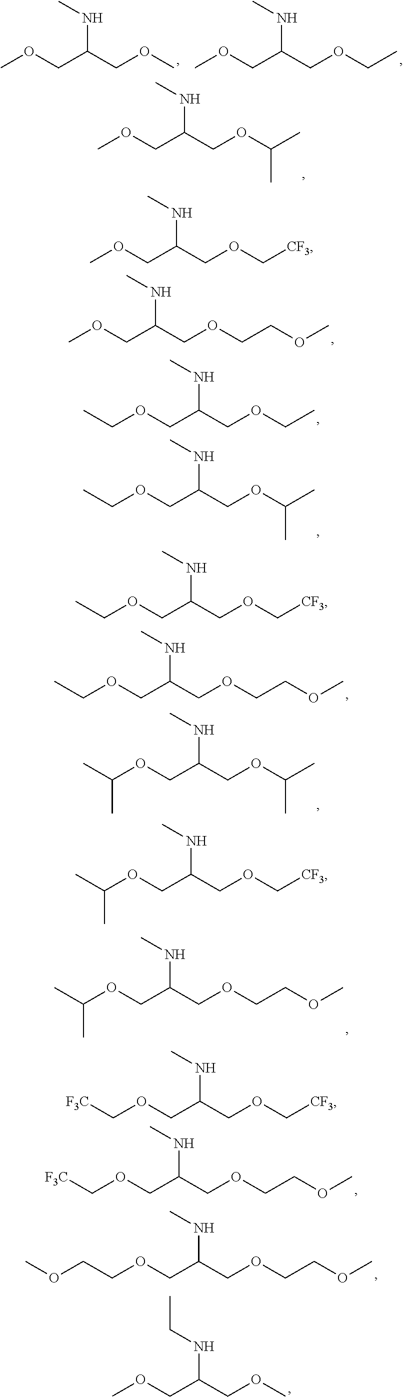

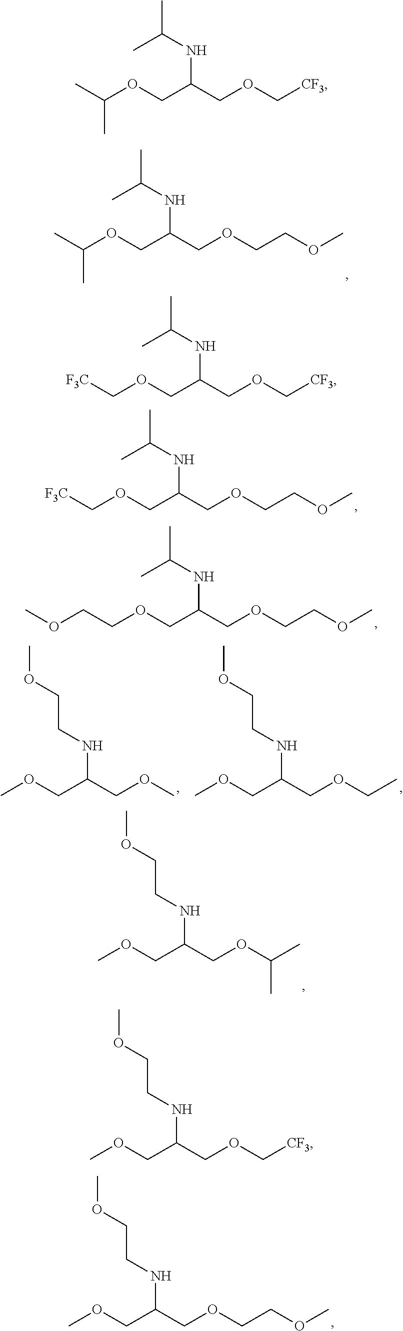

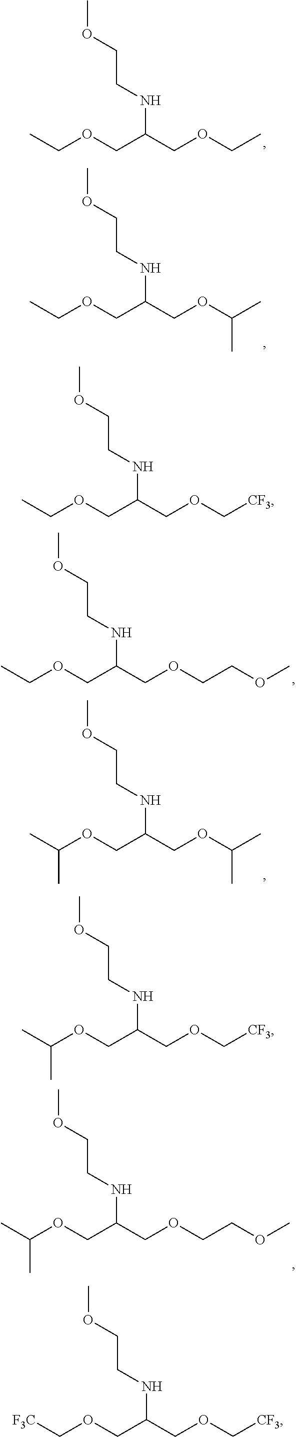

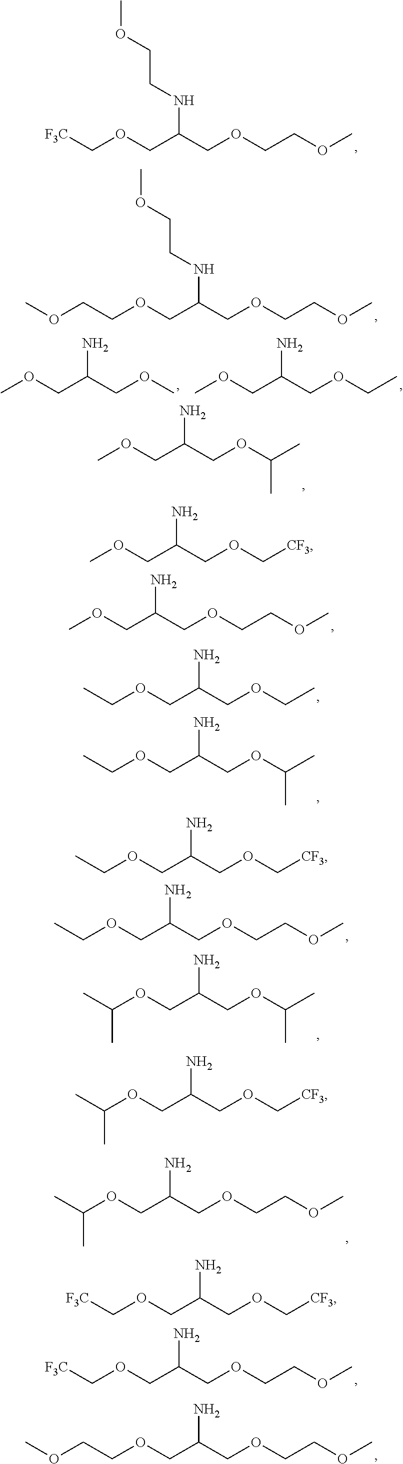

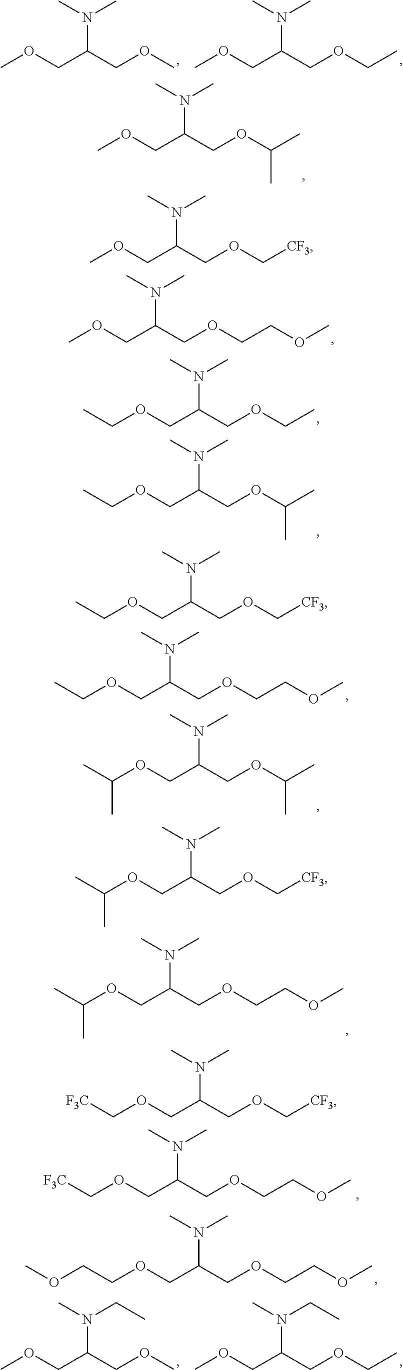

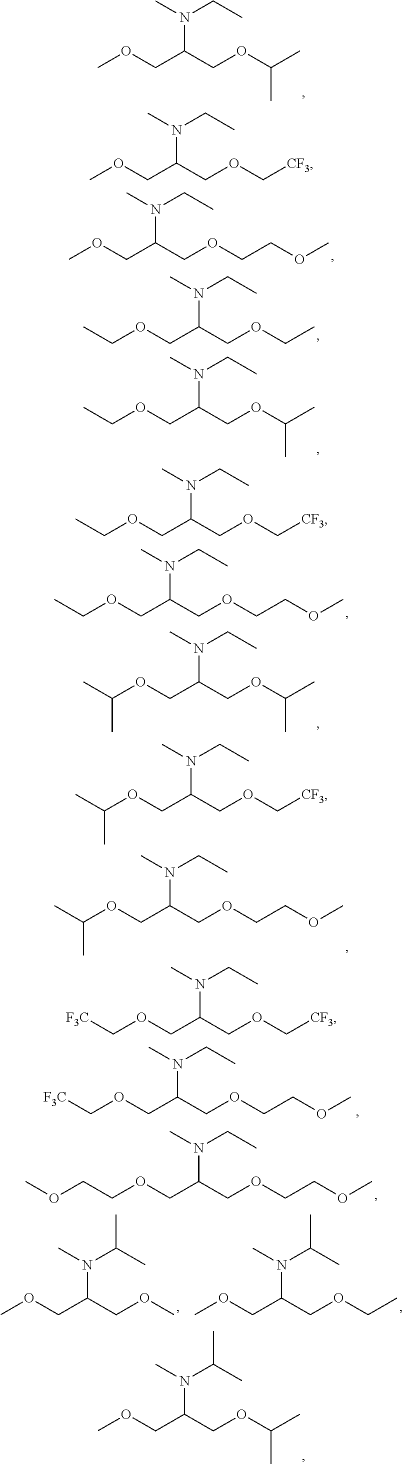

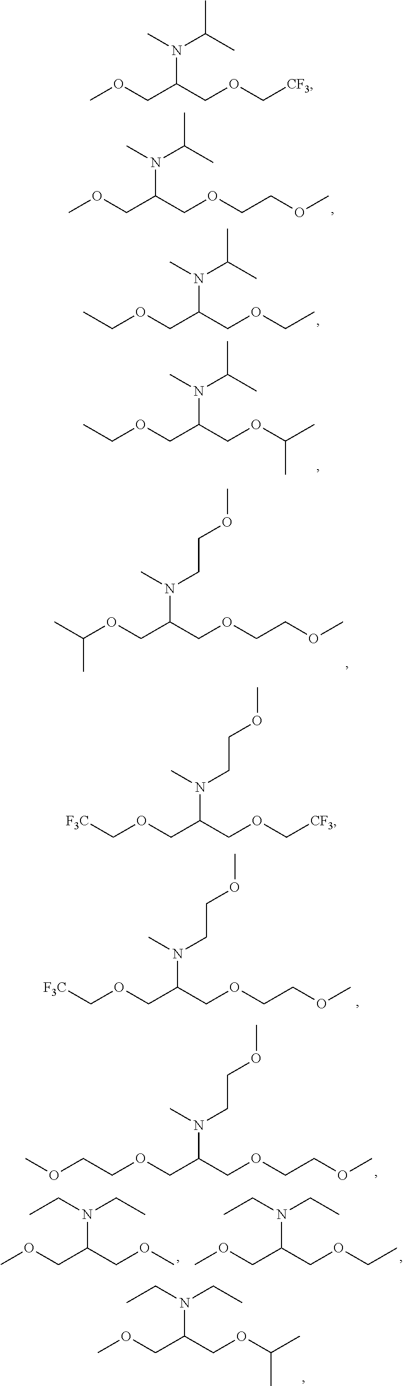

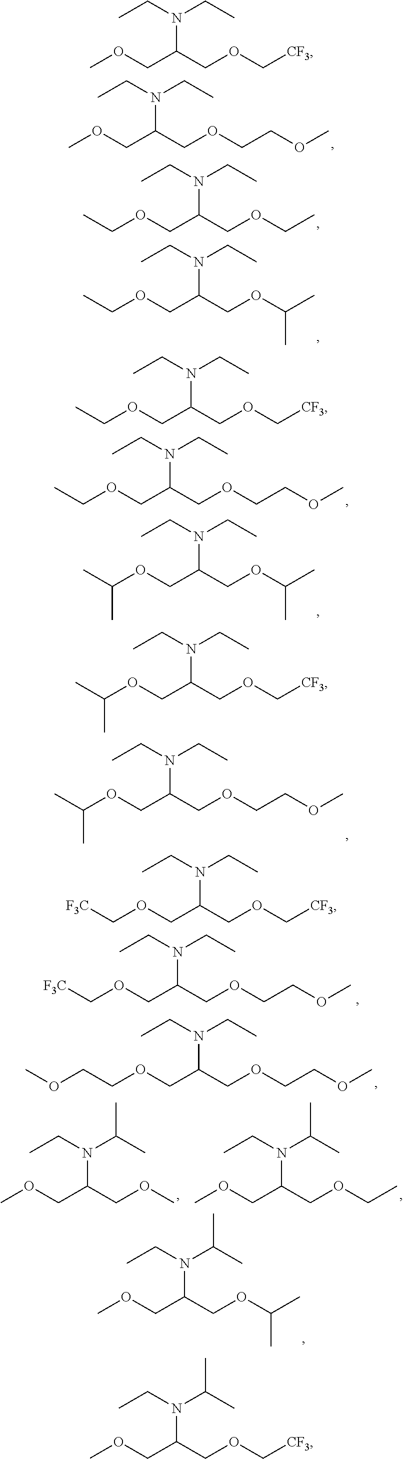

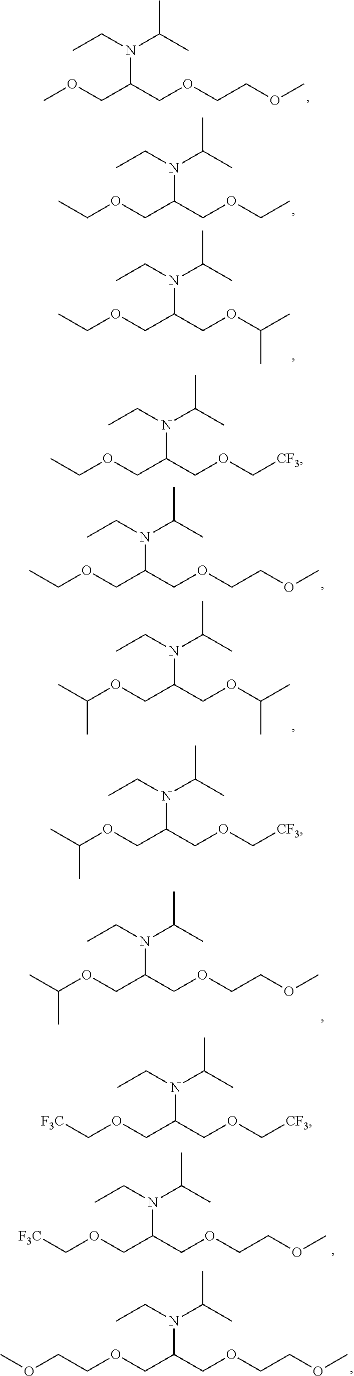

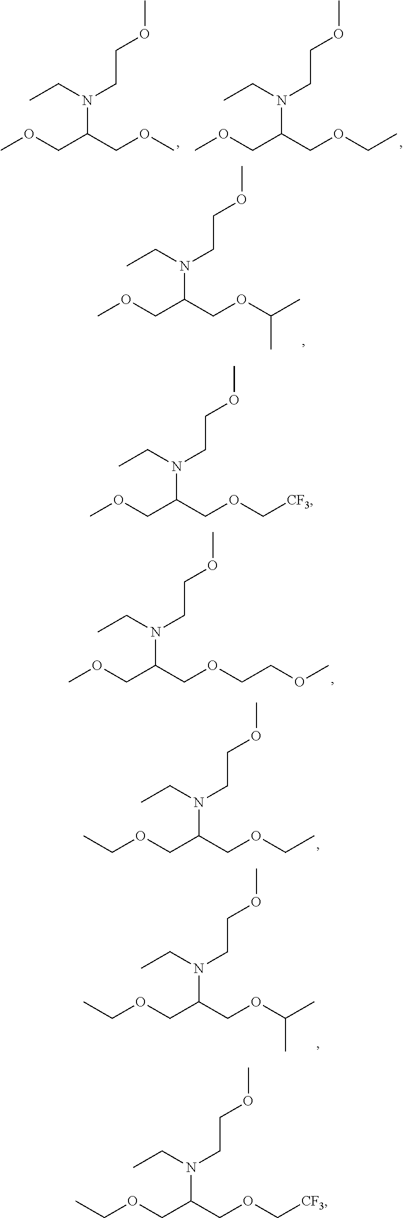

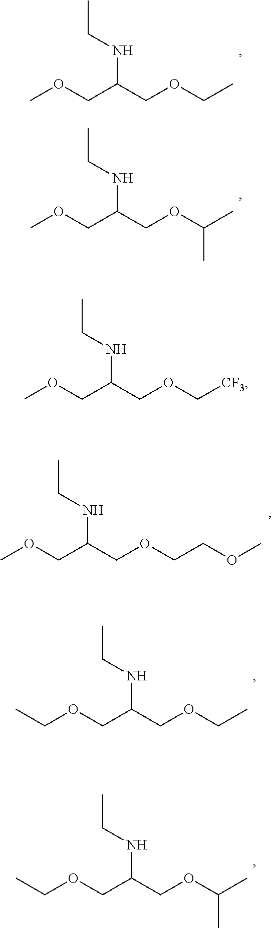

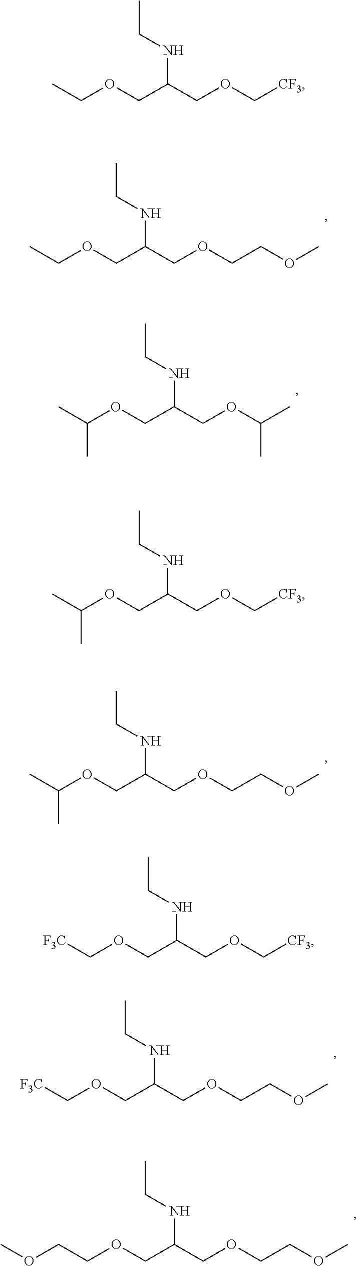

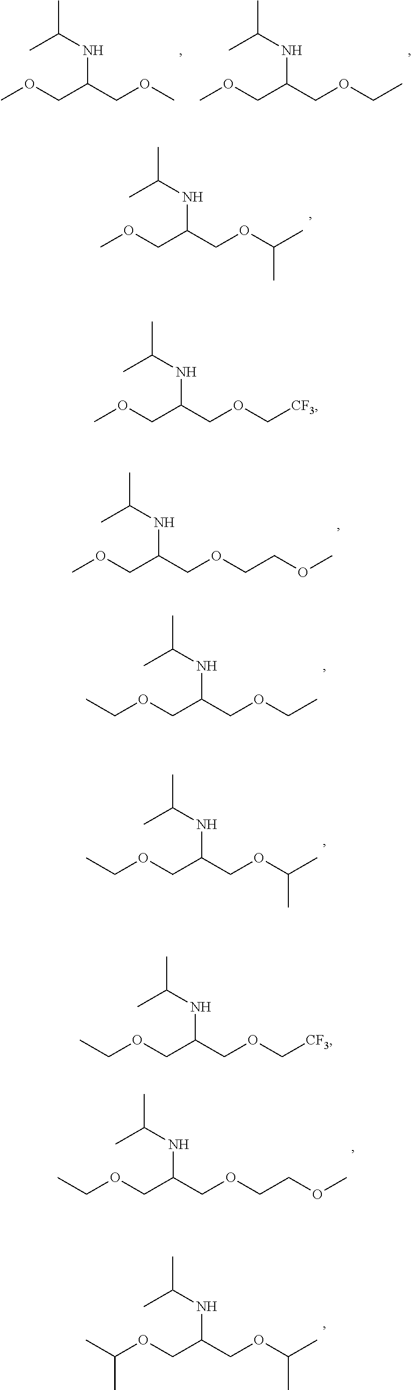

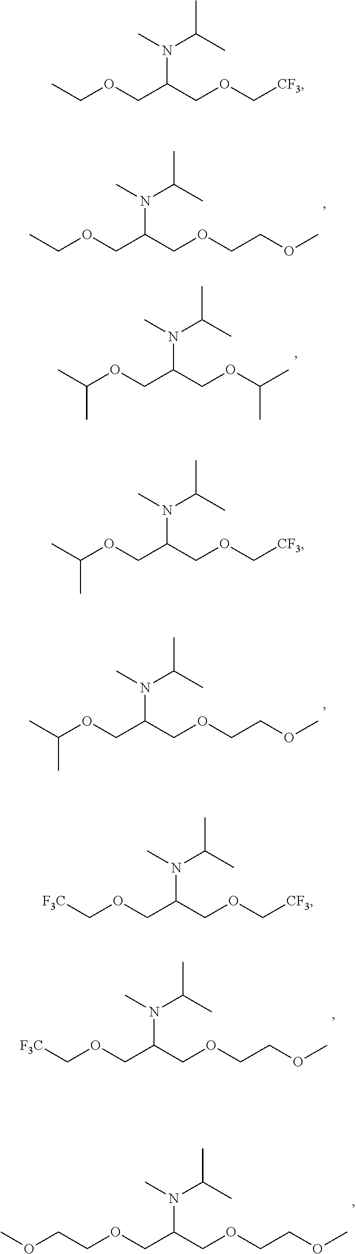

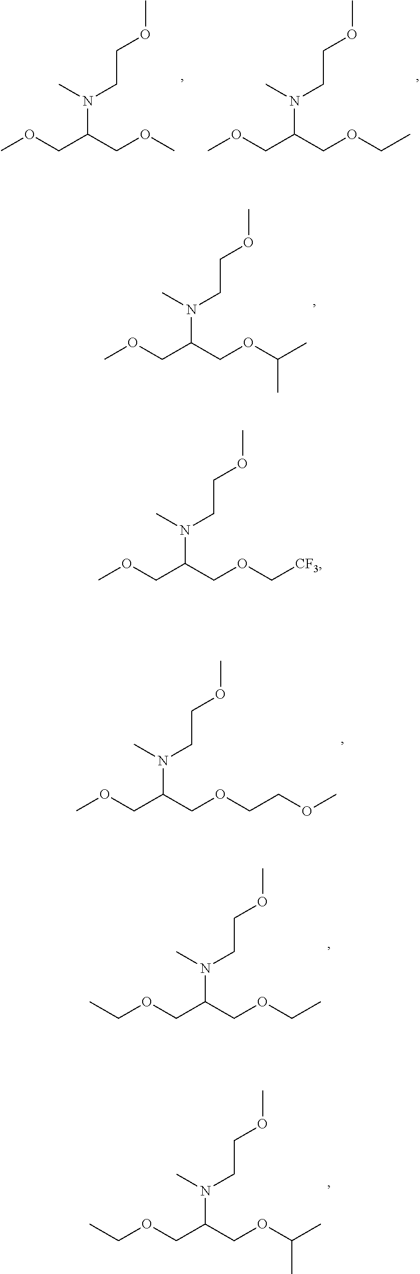

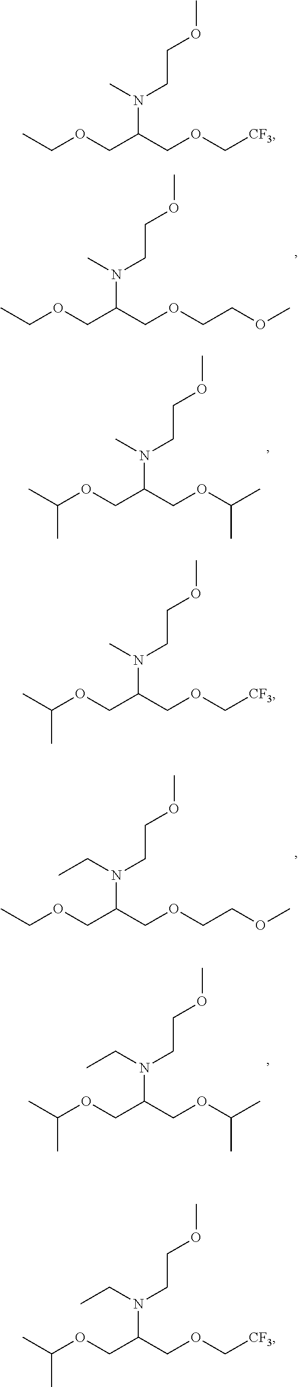

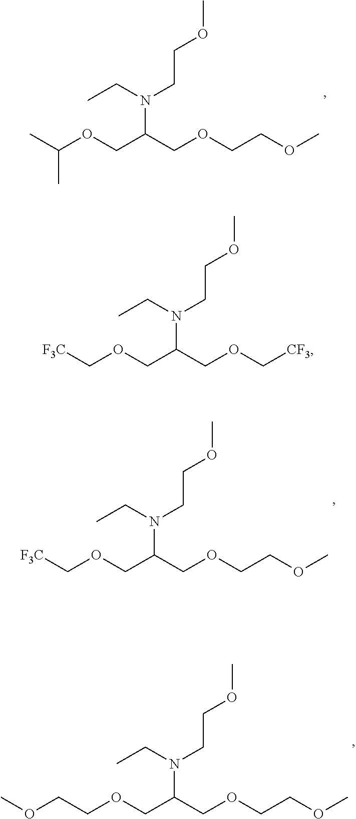

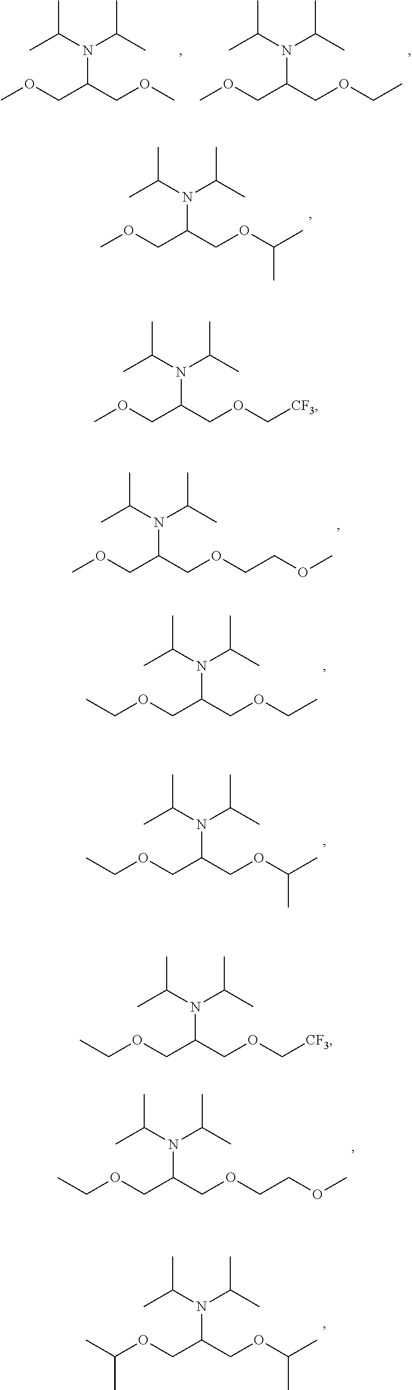

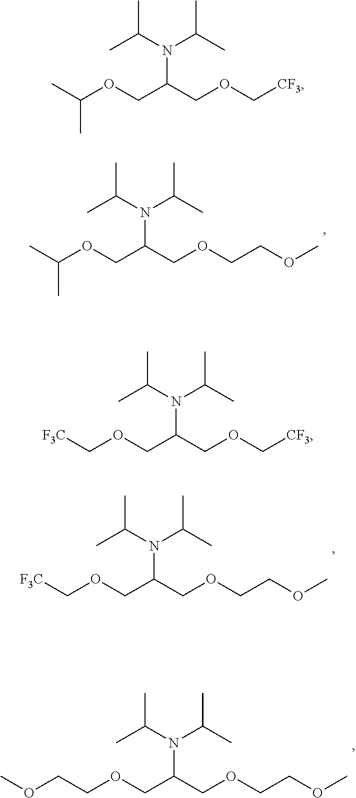

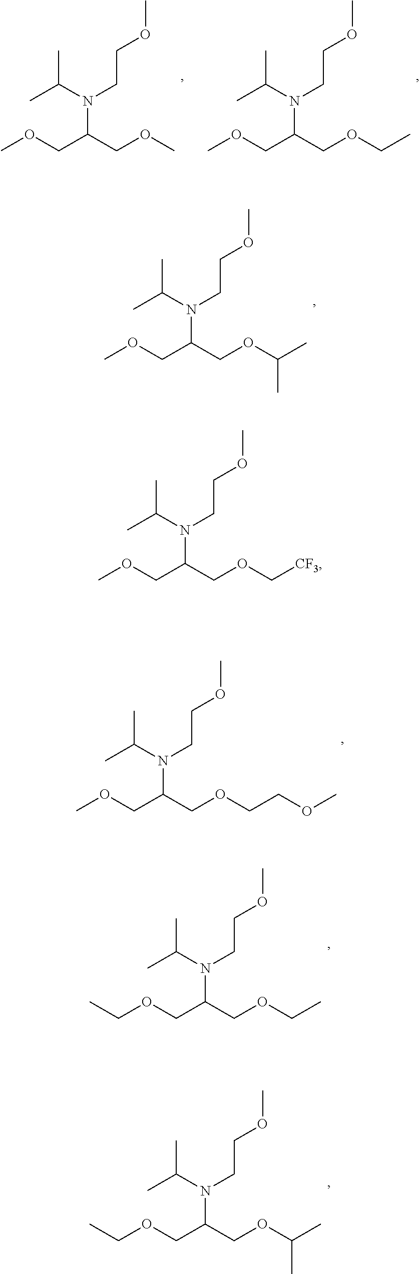

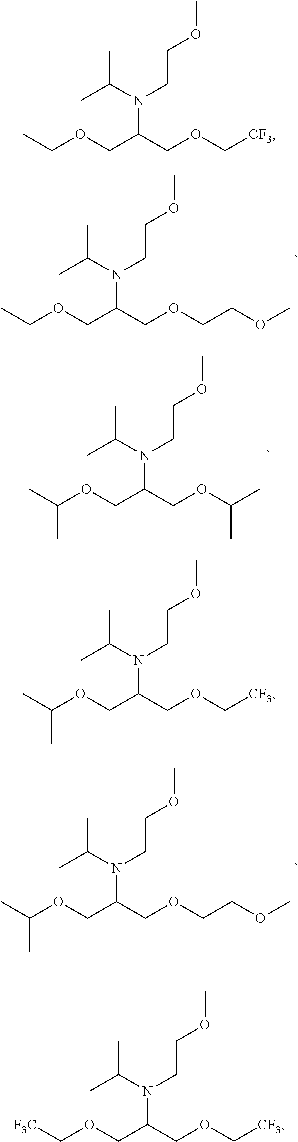

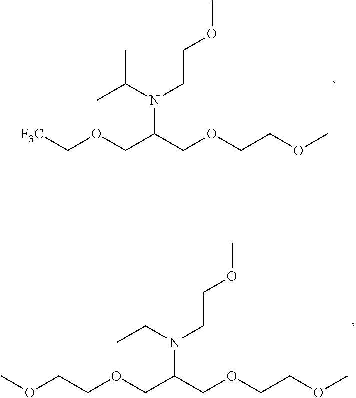

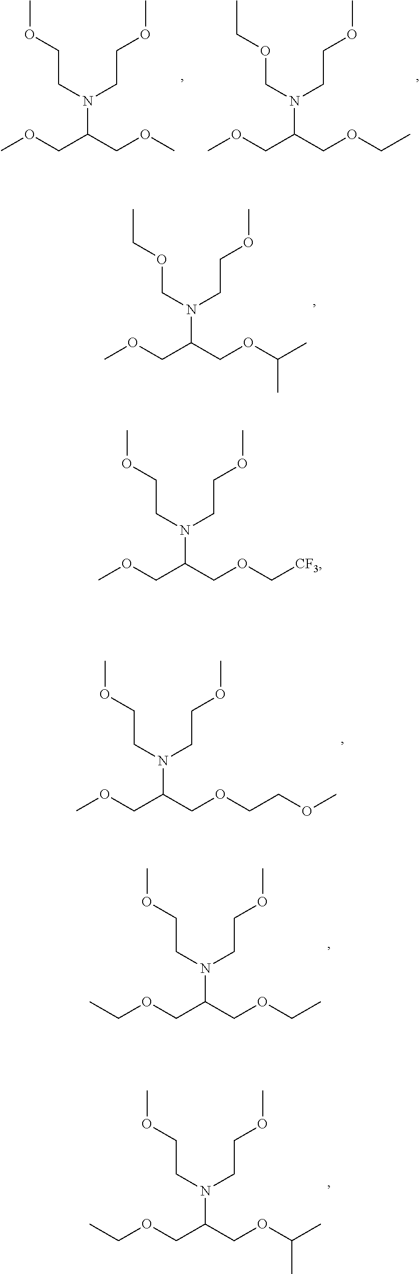

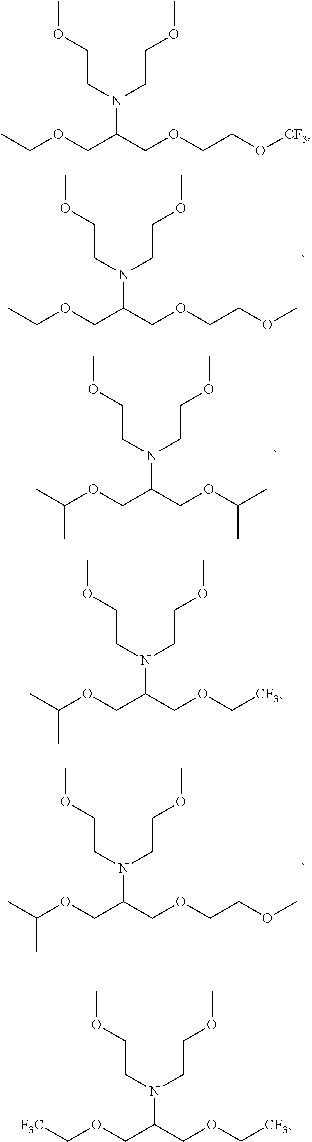

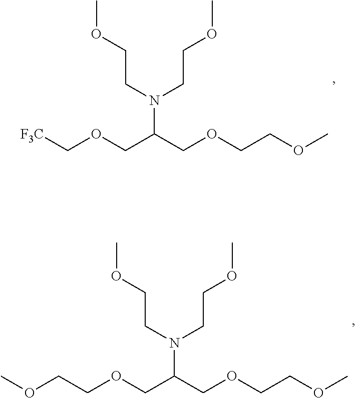

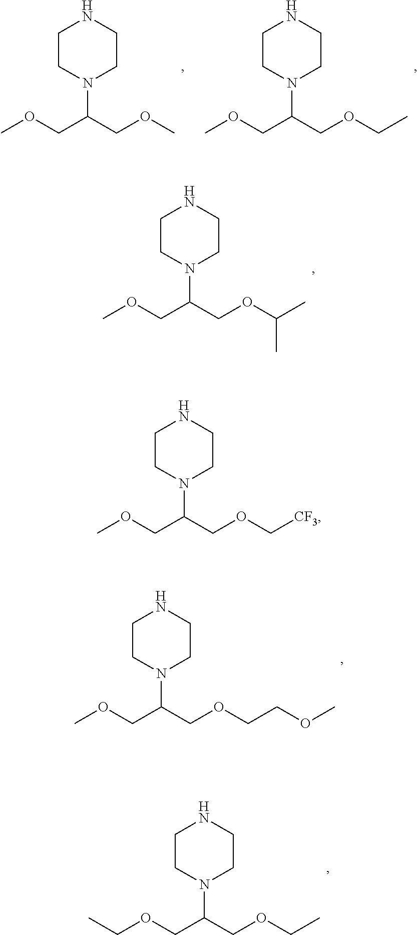

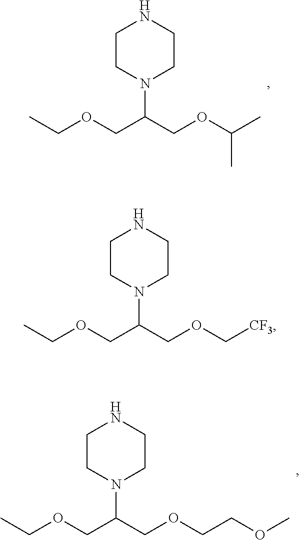

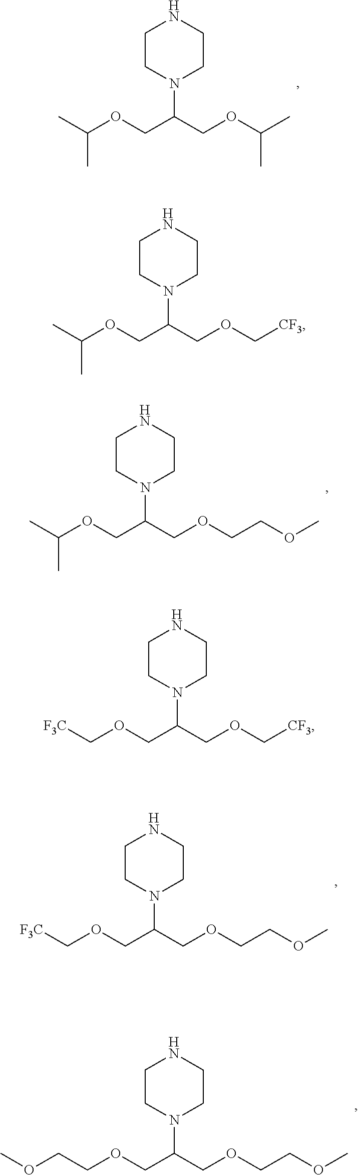

and combinations thereof.

In some examples, the compound is selected from the group consisting of:

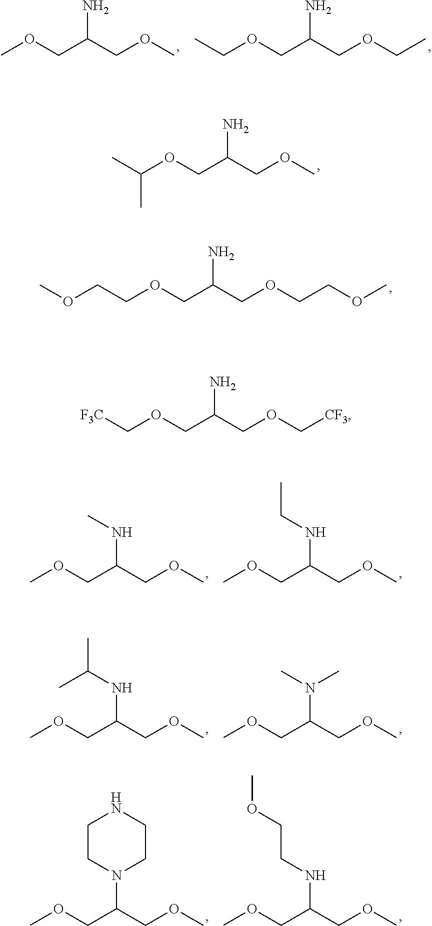

and combinations thereof.

In some examples, the compound has a viscosity of 20 cP or less at 25° C. (e.g., 24 cP or less, 23 cP or less, 22 cP or less, 21 cP or less, 20 cP or less, 19 cP or less, 18 cP or less, 17 cP or less, 16 cP or less, 15 cP or less, 14 cP or less, 13 cP or less, 12 cP or less, 11 cP or less, 10 cP or less, 9 cP or less, 8 cP or less, 7 cP or less, 6 cP or less, 5 cP or less, 4 cP or less, 3 cP or less, 2 cP or less, or 1 cP or less). In some examples, the compound has a viscosity of 5 cP or less at 25° C. (e.g., 4 cP or less, 3 cP or less, 2 cP or less, or 1 cP or less). Viscosity can be measured using methods known in the art, such as with a viscometer or rheometer.

### Solvent System

Also disclosed herein are solvent systems for capturing an acid gas from a fluid stream, the solvent systems comprising any of the compounds disclosed herein (e.g., of Formula I-Formula III).

In some examples, the solvent system further comprises an additional solvent, such as water. In some examples, the solvent system further comprises water in an amount of greater than 0 wt. % (e.g., 1 wt. % or more, 5 wt. % or more, 10 wt. % or more, 15 wt. % or more, 20 wt. % or more, 25 wt. % or more, 30 wt. % or more, 35 wt. % or more, 40 wt. % or more, 45 wt. % or more, 50 wt. % or more, 55 wt. % or more, 60 wt. % or more, 65 wt. % or more, 70 wt. % or more, 75 wt. % or more, 80 wt. % or more, 85 wt. % or more, 90 wt. % or more, or 95 wt. % or more). In some examples, the solvent system further comprises water in an amount of less than 100 wt. % (e.g., 95 wt. % or less, 90 wt. % or less, 85 wt. % or less, 80 wt. % or less, 75 wt. % or less, 70 wt. % or less, 65 wt. % or less, 60 wt. % or less, 55 wt. % or less, 50 wt. % or less, 45 wt. % or less, 40 wt. % or less, 35 wt. % or less, 30 wt. % or less, 25 wt. % or less, 20 wt. % or less, 15 wt. % or less, 10 wt. % or less, 5 wt. % or less, or 1 wt. % or less). The amount of water in the solvent system can range from any of the minimum values described above to any of the maximum values described above. For example, the solvent system can further comprise water in an amount of from greater than 0 wt. % to less than 100 wt. % (e.g., from greater than 0 wt. % to 50 wt. %, from 50 wt. % to less than 100 wt. %, from greater than 0 wt. % to 20 wt. %, from 20 wt. % to 40 wt. %, from 40 wt. % to 60 wt. %, from 60 wt. % to 80 wt. %, from 80 wt. % to less than 100 wt. %, from greater than 0 wt. % to 80 wt. %, from greater than 0 wt. % to 60 wt. %, from greater than 0 wt. % to 40 wt. %, from greater than 0 wt. % to 30 wt. %, from greater than 0 wt. % to 25 wt. %, from greater than 0 wt. % to 10 wt. %, from greater than 0 wt. % to 5 wt. %, from 1 wt. % to less than 100 wt. %, from 5 wt. % to less than 100 wt. %, from 10 wt. % to less than 100 wt. %, from 15 wt. % to less than 100 wt. %, from 20 wt. % to less than 100 wt. %, from 25 wt. % to less than 100 wt. %, from 30 wt. % to less than 100 wt. %, from 40 wt. % to less than 100 wt. %, from 60 wt. % to less than 100 wt. %, from 70 wt. % to less than 100 wt. %, from 90 wt. % to less than 100 wt. %, from 1 wt. % to 99 wt. %, from 5 wt. % to 95 wt. %, from 10 wt. % to 90 wt. %, or from 25 wt. % to 75 wt. %).

In some examples, the solvent system comprises an aqueous solution comprising greater than 25 wt. % of water (e.g., 30 wt. % or more, 35 wt. % or more, 40 wt. % or more, 45 wt. % or more, 50 wt. % or more, 55 wt. % or more, 60 wt. % or more, 65 wt. % or more, 70 wt. % or more, 75 wt. % or more, 80 wt. % or more, 85 wt. % or more, 90 wt. % or more, or 95 wt. % or more). In some examples, the solvent system comprises an aqueous solution comprising less than 100 wt. % of water (e.g., 95 wt. % or less, 90 wt. % or less, 85 wt. % or less, 80 wt. % or less, 75 wt. % or less, 70 wt. % or less, 65 wt. % or less, 60 wt. % or less, 55 wt. % or less, 50 wt. % or less, 45 wt. % or less, 40 wt. % or less, 35 wt. % or less, or 30 wt. % or less). The amount of water in the aqueous solution solvent system can range from any of the minimum values described above to any of the maximum values described above. For example, solvent system comprises an aqueous solution comprising from greater than 25 wt. % to less than 100 wt. % (e.g., from greater than 25 wt. % to 37.5 wt. %, from 37.5 wt. % to less than 100 wt. %, from greater than 25 wt. % to 50 wt. %, from 50 wt. % to 75 wt. %, from 75 wt. % to less than 100 wt. %, from greater than 25 wt. % to 80 wt. %, from greater than 25 wt. % to 60 wt. %, from greater than 25 wt. % to 40 wt. %, from 26 wt. % to less than 100 wt. %, from 30 wt. % to less than 100 wt. %, from 40 wt. % to less than 100 wt. %, from 60 wt. % to less than 100 wt. %, from 70 wt. % to less than 100 wt. %, from 90 wt. % to less than 100 wt. %, from 26 wt. % to 99 wt. %, from 30 wt. % to 95 wt. %, from 40 wt. % to 90 wt. %, or from 50 wt. % to 80 wt. %).

In some examples, the solvent system is water-lean. As used herein, “water-lean” means that the solvent system comprises 25 wt. % or less of water (e.g., 24 wt. % or less, 23 wt. % or less, 22 wt. % or less, 21 wt. % or less, 20 wt. % or less, 19 wt. % or less, 18 wt. % or less, 17 wt. % or less, 16 wt. % or less, 15 wt. % or less, 14 wt. % or less, 13 wt. % or less, 12 wt. % or less, 11 wt. % or less, 10 wt. % or less, 9 wt. % or less, 8 wt. % or less, 7 wt. % or less, 6 wt. % or less, 5 wt. % or less, 4 wt. % or less, 3 wt. % or less, 2 wt. % or less, or 1 wt. % or less). For example, a water-lean solvent system can comprise from 0 wt. % to 25 wt. % water (e.g., from 0 wt. % to 12.5 wt. %, from 12.5 wt. % to 25 wt. %, from 0 wt. % to 20 wt. %, from 0 wt. % to 15 wt. %, from 0 wt. % to 10 wt. %, from 0 wt. % to 5 wt. %, or from 0 wt. % to 1 wt. %). In some examples, the solvent system is substantially free of water.

In some examples, the solvent system comprises 5% or more of the compound by volume (e.g., 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more). In some examples, the solvent system comprises 100% or less of the compound by volume (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, or 10% or less). The amount of the compound in the solvent system can range from any of the minimum values described above to any of the maximum values described above. For example, the solvent system can comprise from 5% to 100% of the compound by volume (e.g., from 5% to 50%, from 50% to 100%, from 5% to 20%, from 20% to 40%, from 40% to 60%, from 60% to 80%, from 80% to 100%, from 5% to 90%, from 5% to 80%, from 5% to 60%, from 5% to 30%, from 5% to 10%, from 10% to 100%, from 20% to 100%, from 30% to 100%, from 40% to 100%, from 60% to 100%, from 70% to 100%, from 10% to 90%, or from 20% to 80%).

In some examples, the solvent system has a viscosity of 20 cP or less at 25° C. (e.g., 24 cP or less, 23 cP or less, 22 cP or less, 21 cP or less, 20 cP or less, 19 cP or less, 18 cP or less, 17 cP or less, 16 cP or less, 15 cP or less, 14 cP or less, 13 cP or less, 12 cP or less, 11 cP or less, 10 cP or less, 9 cP or less, 8 cP or less, 7 cP or less, 6 cP or less, 5 cP or less, 4 cP or less, 3 cP or less, 2 cP or less, or 1 cP or less). In some examples, the solvent system has a viscosity of 5 cP or less at 25° C. (e.g., 4 cP or less, 3 cP or less, 2 cP or less, or 1 cP or less). Viscosity can be measured using methods known in the art, such as with a viscometer or rheometer.

### Methods of Use

Also disclosed herein are methods of use of any of the compounds or solvent systems disclosed herein.

For example, also disclosed herein are methods of using any of the compounds or solvent systems disclosed herein for capturing an acid gas from a fluid steam. In some examples, the methods comprise contacting the compound or solvent system with the fluid stream, wherein the fluid stream comprises the acid gas, thereby capturing the acid gas from the fluid stream.

In some examples, the acid gas is captured by the compound by reacting with the compound to form a product. In some examples, the product has a viscosity of 20 cP or less at 25° C. (e.g., 19 cP or less, 18 cP or less, 17 cP or less, 16 cP or less, 15 cP or less, 14 cP or less, 13 cP or less, 12 cP or less, 11 cP or less, 10 cP or less, 9 cP or less, 8 cP or less, 7 cP or less, 6 cP or less, 5 cP or less, 4 cP or less, 3 cP or less, 2 cP or less, or 1 cP or less). Viscosity can be measured using methods known in the art, such as with a viscometer or rheometer.

In some examples, the methods can further comprise separating the product from the fluid stream, wherein the fluid stream has a reduced amount of the acid gas relative to before the fluid stream was contacted with the compound or the solvent system.

In some examples, the method further comprises regenerating the compound. Regenerating the compound can, for example, comprise exposing the product to steam and/or vacuum to strip the acid gas from the compound. In some examples, the methods can further comprise collecting the acid gas after it has been stripped from the compound.

In some examples, the acid gas comprises carbon dioxide, sulfur dioxide, hydrogen sulfide, or a combination thereof. In some examples, the acid gas comprises CO₂, hydrogen sulfide, or a combination thereof. In some examples, the acid gas comprises CO₂.

In some examples, the fluid stream comprises an industrial gas stream such as a power plant emission (e.g., flue gas), ambient air, or a combination thereof. In some examples, the fluid stream comprises ambient air.

In some examples, the fluid stream comprises ambient air and the acid gas comprises carbon dioxide (e.g., the method comprises direct air capture).

In some examples, the fluid stream comprises natural gas, byproducts of a chemical reaction, post-combustion flue gas, or a combination thereof.

### Methods of Making

Also disclosed herein are methods of making any of the compounds or compositions disclosed herein.

In some examples, the compound is derived at least in part from natural products (e.g., biosourced).

The compounds described herein can be prepared in a variety of ways known to one skilled in the art of organic synthesis or variations thereon as appreciated by those skilled in the art. The compounds described herein can be prepared from readily available starting materials.

Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Katchem (Prague, Czech Republic), Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical excipients disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Serinol Derivatives for CO₂ Capture

Described herein are a new family of molecules that reversibly react with CO₂, hydrogen sulfide, or other acid gases. The molecular structures of this new family of molecules are derived from glycerol, more specifically serinol. The compounds are derived partially from renewable starting materials (e.g., glycerol is abundant and can be biosourced) and are low cost and scalable. The compounds have low viscosity, low vapor pressure, and high CO₂ capacity. The compounds can function with or without added water.

Serinol (Scheme 1) is an amino alcohol analogue of the amino serine. Serinol is identical to glycerol (Scheme 2), except that the central-OH has been replaced by —NH₂. Serinol has limited use in CO₂ capture applications as it is a solid and must be dissolved in water.

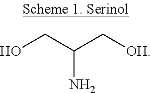

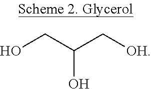

Serinol derivatives (e.g., diethers) can be produced inexpensively via ring opening of epichlorohydrin or glycidyl ethers with alcohol in presence of base, followed by chlorination and reaction with amine (Scheme 3). There is great tunability of physical and chemical properties.

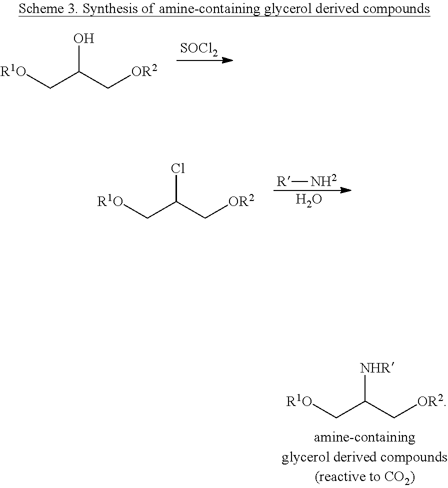

The synthetic approach shown in Scheme 3 enables broad access to new solvents; the R groups give variability. Example serinol derivatives with varying R groups (symmetric or asymmetric) are shown in Scheme 4; tuning these R groups can tune the physical properties of the solvents. Example serinol derivatives with varying amine groups are shown in Scheme 5; turning the amine group can tune the chemical properties of the solvents.

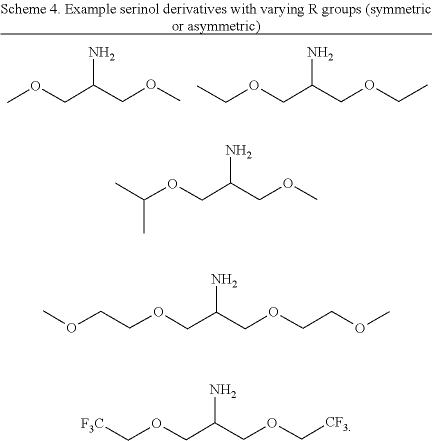

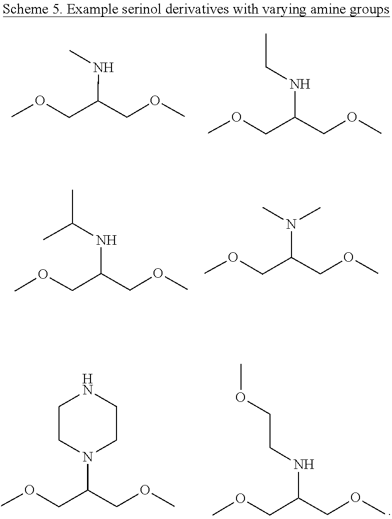

These new compounds are attractive for “water-lean” CO₂ capture solvents. The interest in “water-lean” CO₂ capture solvents is growing. For example, there has been extensive work recently on a highly promising single component water-lean solvent, N-(2-ethoxyethyl)-3-morpholinopropan-1-amine (EEMPA).

The new compounds can provide improved combinations of properties for water lean CO₂ capture solvents. The new compounds are inexpensive (raw materials are low cost) and the synthesis is scalable (e.g., could move to multi-gallon pilot testing).

Structure-property relationships can be investigated. Based on this, candidates can be identified for further scale-up.

U.S. Pat. No. 11,452,969 describes non-reactive solvents for CO₂ capture applications (e.g., there is no chemical reaction between the solvent and CO₂, only a physical interaction). Meanwhile, the compounds described herein are reactive solvents for CO₂ capture applications. The compounds described herein can reversibly react with CO₂, hydrogen sulfide, or other acid gases. For example, a reactive solvent is needed when the concentration (or partial pressure) of CO₂ (or other acid gas) is low, such as in power plant emissions and/or for direct air capture.

Data herein suggests that the new compounds described herein will be superior to other CO₂ capture compounds described elsewhere.

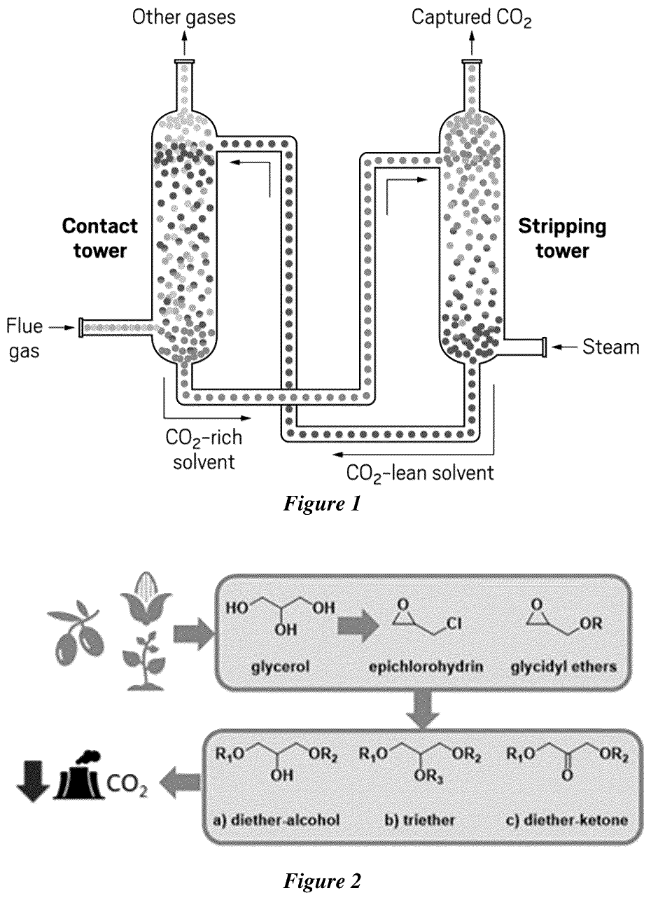

An example schematic of CO₂ capture process operation is shown in FIG. 1. A CO₂ gas (such as a flue gas) enters the system as shown. In the contact tower, there is a counter current flow of the solvent. Contact between the solvent and the CO₂ in the capture tower leads to capture of the CO₂ and the CO₂-rich solvent exits to the stripping tower, while other gases escape from the contact tower. In the stripping tower, CO₂ is stripped from the solvent, for example using steam and/or vacuum. The captured CO₂ can then exit from the stripping tower, while the solvent can be recycled back to the contact tower. Improved CO₂ capture solvents allow for better capture efficiency and therefore a smaller footprint of the CO₂ capture process.

### Example 2—Serinol Derivatives for CO₂ Capture

Described herein are new compounds derived from glycerol (or precursors with similar structures, such as serinol) that contain amine functional groups that can react with CO₂, and can therefore be used to capture CO₂ from industrial gas streams. In some examples, the new compounds are derived (at least in part) from natural products. They can operate with or without added water or co-solvent.

For example, the compounds can be used to capture CO₂ from power plants, industrial gas streams, and even ambient air. They can also have use as “switchable solvents”, which will be beneficial for the processing of biomass and other chemicals.

### Example 3

CO₂ Capture: One of the greatest current challenges is mitigating climate change caused by greenhouse gas (CO₂, CH₄, etc.) emissions. Combustion of coal and fossil fuels has led to anthropogenic CO₂ emissions of >37 GT/y and has resulted in high CO₂ levels in the atmosphere (>410 ppm; partial pressure of ˜4×10⁻⁴ bar as of 2019) with corresponding increases in ocean acidity.

Absorptive technologies (i.e., solvents) are the most widely used and mature processes for CO₂ removal from industrial gas streams, with documented use over 90+ years. Well before the relatively recent thrusts to capture CO₂ from power plant emissions and direct air capture, removal of CO₂ from raw natural gas to levels below 2 vol % has been necessary to limit pipeline corrosion and maintain heating value. Separation of CO₂ is also a key component of water-gas shift reactions which produce H₂ and CO₂ from CO and H₂O, which is important to refineries and NH₃ manufacture, and electric power generation via integrated gasification combined cycle (IGCC) processes.

The selection of an appropriate solvent for CO₂ removal is related to the inlet partial pressure and outlet purity specification (Tennyson R N et al. Oil & Gas Journal 1977, 75 (2), 78-86). When the inlet pressure is high and only “bulk” (˜90%) removal of CO₂ is required, a non-reactive “physical” solvent is used (Tennyson R N et al. Oil & Gas Journal 1977, 75 (2), 78-86). Physical solvents are typically polar organic solvents with low viscosity, low vapor pressure, and thermal stability (Bucklin R W et al. Energy Progress 1984, 4 (3), 137-142; Lin H et al. Journal of Molecular Structure 2005, 739 (1-3), 57-74). The amount of CO₂ absorbed by a physical solvent is related to the partial pressure of CO₂ in the gas stream, typically a Henry's Law relationship. Because there are no chemical bonds are formed/broken, physical solvents generally require only mild heating and/or vacuum (flash) stripping to release CO₂ (i.e., relatively low energy requirements).

Among physical solvent processes, Selexol® is perhaps the most widely used. Selexol utilizes dimethyl ethers of poly(ethylene glycol) (DMPEG) as the working fluid, as a high boiling point (275° C.) and moderate viscosity (5.8 cP at 20° C.). DMPEG is produced from the polymerization of (petroleum-derived) ethylene oxide (EO). EO is itself produced on massive scales (>20 million metric tons (t)/yr) (Bhown A S et al. Environ. Sci. Technol. 2011, 45 (20), 8624-8632). The average number of —(CH₂CH₂O)— repeat units in the linear DMPEG molecules is ˜6, (where a distribution of species with 4-10 units is present). Glycerol-derived compounds (FIG. 2) are chemically similar to DMPEG, and thus glycerol-derived compounds that perform as least as well as DMPEG will be more sustainable and could be “drop-in” (i.e., requiring little/no process modification) replacements for DMPEG in CO₂ capture processes (Qian S et al. Fluid Phase Equilib. 2020, 521, 112718; Qian S et al. Industrial & Engineering Chemistry Research 2020, 59 (45), 20190-20200; Flowers B S et al. ACS Sustainable Chemistry & Engineering 2017, 5 (1), 911-921). The triether species (FIG. 2) have recently been added to GSK's “Solvent Sustainability Guide” (Alder C M et al. Green Chemistry 2016, 18 (13), 3879-3890) and are considered to be much less toxic than glymes within DMPEG (Garcia J I et al. Green Chemistry 2015, 17 (8), 4326-4333).

U.S. Pat. No. 11,452,969 relates to the use of glycerol-derived compounds in gas treating processes. Through combined experimental and computational efforts, glycerol-derived compounds have been studied and it has been demonstrated that they are superior to DMPEG as physical solvents for CO₂ absorption with respect to CO₂ capacity and key physical properties (e.g., viscosity) (Qian S et al. Fluid Phase Equilib. 2020, 521, 112718; Qian S et al. Industrial & Engineering Chemistry Research 2020, 59 (45), 20190-20200; Flowers B S et al. ACS Sustainable Chemistry & Engineering 2017, 5 (1), 911-921; Qian S et al. Chem. Eng. Sci. 2022, 248, 117150; Qian S et al. Aiche J. 2022, 68 (3), e17533). Knowledge of glycerol-derived compounds as physical solvents is further expanded through new molecular designs and measurements on mixed gas streams.

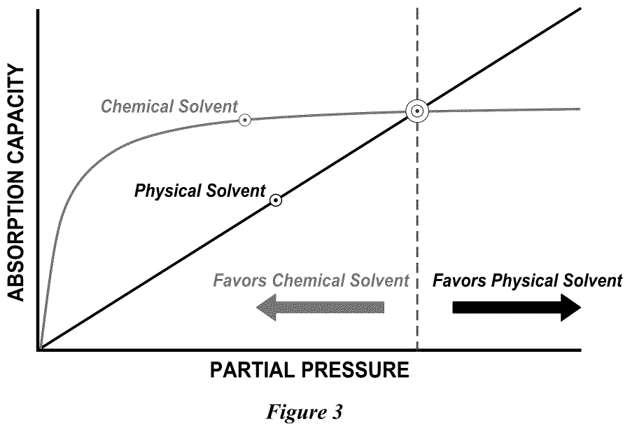

When the inlet partial pressure of CO₂ is low (e.g., power plant flue gas), then a reactive “chemical” solvent containing primary (1°) or secondary (2°) amines is required. The “classical” chemical solvent for CO₂ is 30 wt % aqueous monoethanolamine (aq. MEA). The CO₂ capacity of a chemical solvent is primarily governed by the amine concentration (e.g., molarity), and once the active species is saturated with CO₂, increased CO₂ pressure will result in only minimal additional absorption. As chemical are formed/broken in these solvents, elevated temperatures are required to release the CO₂ and regenerate the solvent. Thus, while chemical solvents can absorb large amounts of CO₂ even from low pressure streams, the regeneration process is more energy intensive, generally requiring steam stripping is generally required. FIG. 3 illustrates the general relationship between CO₂ partial pressure and CO₂ absorption capacity for chemical and physical solvents (NETL. DOE/NETL Advanced Carbon Dioxide Capture R & amp;D Program: May 2013 Update. 2013. https://www.netl.doe.gov/File % 20Library/Research/Coal/carbon % 20capture/handbook/CO₂-Capture-Tech-Update-2013.pdf (accessed 15 Sep. 2016). Under certain conditions, hybrid solvents containing amines, water and a polar organic solvent (e.g., sulfolane) are also employed in CO₂ capture processes, such as Shell's Sulfinol® (Murrieta-Guevara F et al. Fluid Phase Equilib. 1988, 44 (1), 105-115). In recent years, “water-lean solvents” have emerged as highly promising candidates for post-combustion CO₂ capture (Heldebrant D J et al. Chem. Rev. (Washington, DC, U. S.) 2017, 117 (14), 9594-9624; Leclaire J et al. Green Chem. 2018, 20 (22), 5058-5081; Heldebrant D J et al. Energy Procedia 2017, 114, 756-763).

Water-lean solvents offer an alternative to the energy intensive aq. amine solvents, as the general absence of water imparts a benefit of lower specific heat, reducing energy inputs and the overall capture costs (Heldebrant D J et al. Chem. Rev. (Washington, DC, U. S.) 2017, 117 (14), 9594-9624). Examples of water-lean solvents systems include ionic liquids (ILs) (Ramdin M et al. Industrial & Engineering Chemistry Research 2012, 51 (24), 8149-8177; Bara J E. 11-Ionic liquids for post-combustion CO₂ capture A2-Feron, Paul H. M. In Absorption-Based Post-combustion Capture of Carbon Dioxide, Woodhead Publishing, 2016; pp 259-282; Cantu D C et al. The Journal of Physical Chemistry Letters 2016, 7 (9), 1646-1652; Camper D et al. Industrial & Engineering Chemistry Research 2008, 47 (21), 8496-8498), aminosilicones (Perry R J et al. Energy Fuels 2012, 26 (4), 2512-2517; Perry R J et al. Energy & Fuels 2011, 25 (4), 1906-1918; Perry R J et al. ChemSusChem 2010, 3 (8), 919-930), siloxy- and silylamines (Blasucci V M et al. Fluid Phase Equilib. 2010, 294 (1-2), 1-6; Blasucci V et al. Fuel 2010, 89, 1315-1319; Blasucci V et al. Chemical Communications 2009, (1), 116-118), non-aqueous solvents (Shannon M S et al. Separation Science and Technology 2012, 47 (2), 178-188; Shannon M S et al. Industrial & Engineering Chemistry Research 2011, 50 (14), 8665-8677), and “binding” organic liquids (Heldebrant D J et al. Energy & Environmental Science 2008, 1 (4), 487-493; Cantu D C et al. Green Chemistry 2016, 18 (22), 6004-6011; Malhotra D et al. ChemSusChem 2017, 10 (3), 636-642). While promising in the lab, the challenges of managing multi-component solvents in continuous process has directed focus towards single-component water-lean solvents.

Heldebrant and co-workers have developed a highly promising single component water-lean solvent, N-(2-ethoxyethyl)-3-morpholinopropan-1-amine (EEMPA) (Scheme 6), thoroughly characterizing its properties and CO₂ capture performance thorough series of studies, and developed performance and cost estimates through process modeling (Zheng R et al. Energy & Environmental Science 2020, 13 (11), 4106-4113). Technoeconomic reports indicate that implementation of EEMPA for CO₂ capture processes will dramatically reduce total capture cost. While this validates the viability of single-component water-lean solvents, EEMPA has little ability to be modified or tailored. Given the promise of EEMPA and water-lean solvents for post-combustion CO₂ capture, the versatility of glycerol-derived compounds are utilized herein to study new water-lean solvents with superior physical properties, performances, and economics that will greatly expand capabilities for enhancing decarbonization.

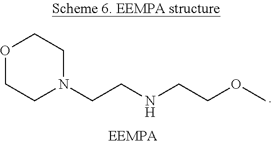

Negative emissions technologies, such as reforestation and direct air capture, can reduce net CO₂ emissions as well as eventually serve to reverse climate change once emissions are stabilized. A recent National Academies report concluded that direct air capture and carbon mineralization have high potential capacity for removing carbon, but direct air capture is currently limited by high cost and carbon mineralization by a lack of fundamental understanding (National Academies of Sciences, E. a. M. Negative Emissions Technologies and Reliable Sequestration: A Research Agenda; The National Academies Press, 2019. DOI: 10.17226/25259). The difficulty is that although the partial pressure of CO₂ is relatively high for its impact on the greenhouse effect, it is still relatively low when considering conventional engineering separation processes such as absorption and adsorption. Successful deployment of direct air capture will require improved materials and a convergent research approach. Glycerol-derived compounds for direct air capture are investigated herein.

Amine-containing glycerol-derived compounds as Water-Lean Solvents for CO₂ Capture: Glycerol-derived compound precursors are provided from which new amine-containing glycerol-derived compound molecules are developed. Recently, the synthesis of glycerol-derived compound diether-chlorides via reaction of diether-alcohols with SOCl₂ was reported (Chatterjee S et al. Industrial & Engineering Chemistry Research 2023. DOI: 10.1021/acs.iecr.2c03441), which creates the opportunity to impart amine functionalities to glycerol-derived compounds for use as water-lean solvents for CO₂ capture from low pressure streams.

Felder utilized glycerol-derived compound 1,3-diether-2-chlorides for the large-scale industrial manufacture of serinol (2-amino-1,3-propanediol) and serinol derivatives (U.S. Pat. No. 4,503,252 A). Serinol is an amino alcohol and structurally analogous to serine (an α-amino acid) (Andreeßen B et al. AMB Express 2011, 1 (1), 12). Serinol is identical to glycerol except that-NH₂ replaces the —OH at the central carbon of glycerol. While serinol shows promise for CO₂ capture processes (Bougie F et al. Journal of Chemical & Engineering Data 2014, 59 (2), 355-361; Abdul Samat N F N et al. J. Mol. Liq. 2019, 277, 207-216), its high price and limited availability make it impractical for large scale use. Reaction of 2-chloro-1,3-diether intermediates with excess ammonia (or organic amines) yields 2-amino-1,3-diethers (Scheme 7), which form the basis of new water-lean solvents for CO₂ capture. These water-lean solvents will be studied along the same lines of prior work with imidazole-amine solvents (Shannon M S et al. Separation Science and Technology 2012, 47 (2), 178-188; Shannon M S et al. Industrial & Engineering Chemistry Research 2011, 50 (14), 8665-8677), as well as the water-lean solvents developed by Heldebrant and co-workers (Heldebrant D J et al. Chem. Rev. (Washington, DC, U. S.) 2017, 117 (14), 9594-9624; Leclaire J et al. Green Chem. 2018, 20 (22), 5058-5081; Heldebrant D J et al. Energy Procedia 2017, 114, 756-763; Zhang J et al. Energy Procedia 2013, 37, 285-291; Mathias P M et al. Energy Environ. Sci. 2013, 6 (7), 2233-2242; Koech P K et al. RSC Adv. 2013, 3 (2), 566-572; Jessop P G et al. Energy Environ. Sci. 2012, 5 (6), 7240-7253; Rainbolt J E et al. Energy & Environmental Science 2011, 4 (2), 480-484; Heldebrant D J et al. Energy Procedia 2011, 4, 216-223; Heldebrant D J et al. Chemical Engineering Journal 2011, 171 (3), 794-800) and others (Blasucci V et al. Fuel 2010, 89, 1315-1319; Jessop P G et al. Energy Environ. Sci. 2012, 5 (6), 7240-7253; Wang C et al. Green Chemistry 2010, 12 (5), 870-874; Wang C et al. Green Chemistry 2010, 12 (11), 2019-2023; Heldebrant D J et al. Chem.-Eur. J. 2009, 15 (31), 7619-7627; Huang J et al. J. Mol. Catal. A: Chem. 2008, 279 (2), 170-176; Yamada T et al. Chemistry of Materials 2007, 19 (5), 967-969; Yamada T et al. Chemistry of Materials 2007, 19 (19), 4761-4768). These amine-containing glycerol-derived compounds have great potential for CO₂ capture given their green origins, scalable manufacture, tunable chemical and physical properties, and ability to function without water. As the amine-containing glycerol-derived compounds are far more tunable than other water-lean solvents, low-cost, highly efficient, stable water-learn solvents can be produced for post-combustion CO₂ capture that maintain low viscosity (<20 cP) in CO₂-rich states.

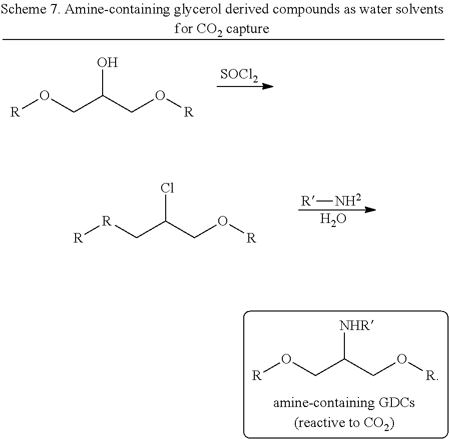

### EXEMPLARY ASPECTS

In view of the described compositions, devices, systems, and methods, herein below are described certain more particularly described aspects of the inventions. The particularly recited aspects should not, however, be interpreted to have any limiting effect on any different claims containing different or more general teachings described herein or that the “particular” aspects are somehow limited in some way other than the inherent meanings of the language and formulas literally used therein.

Example 1: A compound for capturing an acid gas from a fluid stream, wherein the compound is defined by Formula I:

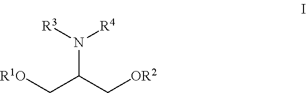

wherein R¹ and R² are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy; and R³ and R⁴ are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy, or wherein, as valence permits, R³ and R⁴, together with the atoms to which they are attached, form a 3-10 membered substituted or unsubstituted cyclic moiety, optionally including 1 to 3 additional heteroatoms; with the proviso that at least one of R¹-R⁴ is not H.

Example 2: The compound of any examples herein, particularly example 1, wherein: R¹ and R² are each independently substituted or unsubstituted C₁₋₂₀ alkyl, or substituted or unsubstituted C₁-C₂₀ alkoxy; and R³ and R⁴ are each independently H, substituted or unsubstituted C₁₋₂₀ alkyl, or substituted or unsubstituted C₁-C₂₀ alkoxy, or wherein, as valence permits, R³ and R⁴, together with the atoms to which they are attached, form a 3-10 membered substituted or unsubstituted cyclic moiety, optionally including 1 to 3 additional heteroatoms.

Example 3: The compound of any examples herein, particularly example 1 or example 2, wherein: R¹ and R² are each independently substituted or unsubstituted C₁₋₆ alkyl, or substituted or unsubstituted C₁-C₆ alkoxy; and R³ and R⁴ are each independently H, substituted or unsubstituted C₁₋₆ alkyl, or substituted or unsubstituted C₁-C₆ alkoxy, or wherein, as valence permits, R³ and R⁴, together with the atoms to which they are attached, form a 6 membered substituted or unsubstituted cyclic moiety, optionally including 1 additional heteroatom.

Example 4: The compound of any examples herein, particularly examples 1-3, wherein: R¹ and R² are each independently methyl, ethyl, isopropyl, —CH₂CF₃, —CH₂CH₂OCH₃; and R³ and R⁴ are each independently H, methyl, ethyl, isopropyl, or —CH₂CH₂OCH₃, or R³ and R⁴, together with the atoms to which they are attached, form a piperazine.

Example 5: The compound of any examples herein, particularly examples 1-4, wherein R³ and R⁴ are the same.

Example 6: The compound of any examples herein, particularly examples 1-4, wherein R³ and R⁴ are different.

Example 7: The compound of any examples herein, particularly examples 1-6, wherein R⁴ is hydrogen.

Example 8: The compound of any examples herein, particularly examples 1-7, wherein the compound is defined by Formula II:

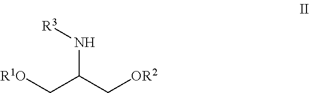

wherein R¹ and R² are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy; and R³ is H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy, or wherein, as valence permits, R³ and R⁴, together with the atoms to which they are attached, form a 3-10 membered substituted or unsubstituted cyclic moiety, optionally including 1 to 3 additional heteroatoms; with the proviso that at least one of R¹-R³ is not H.

Example 9: The compound of any examples herein, particularly examples 1-8, wherein R³ is methyl, ethyl, isopropyl, or —CH₂CH₂OCH₃.

Example 10: The compound of any examples herein, particularly examples 1-9, wherein the compound is selected from the group consisting of:

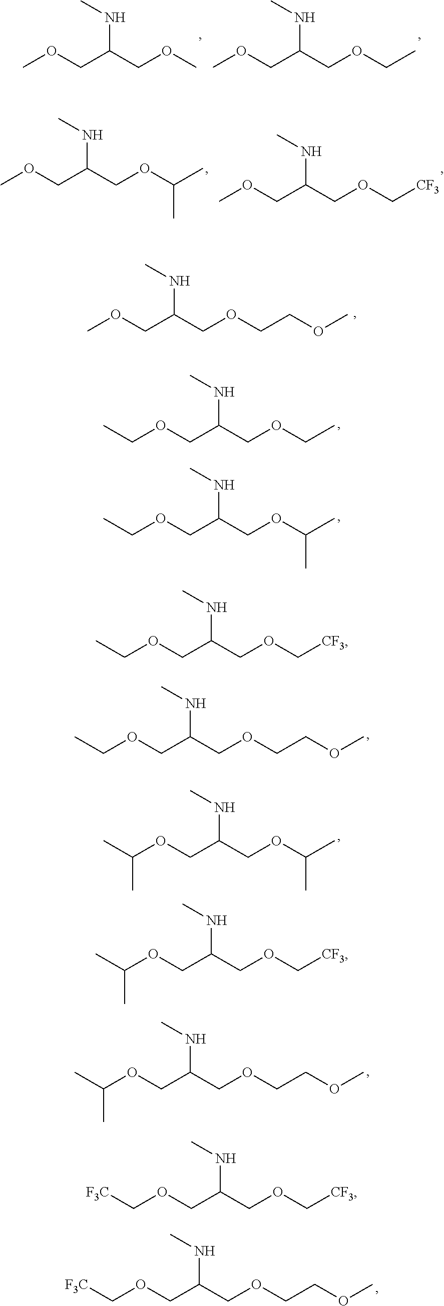

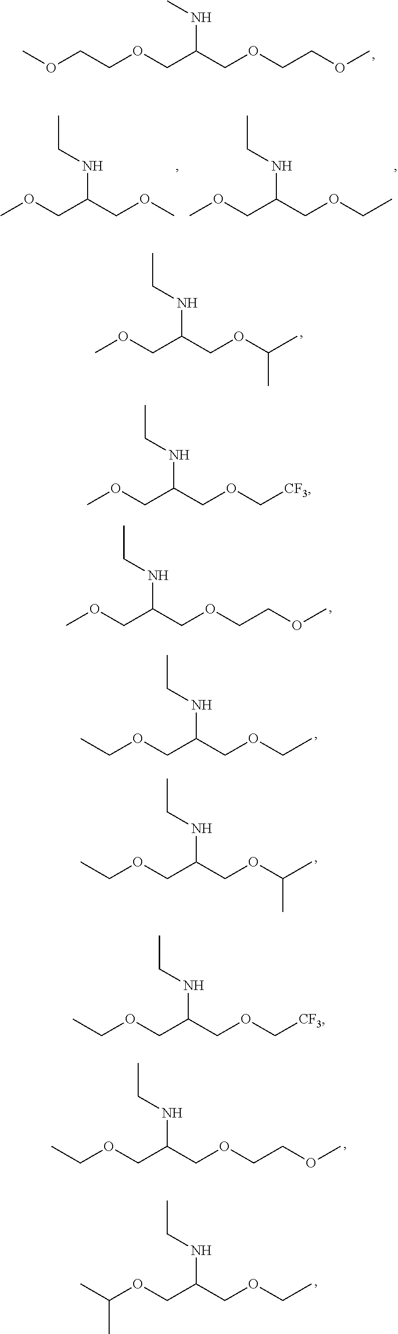

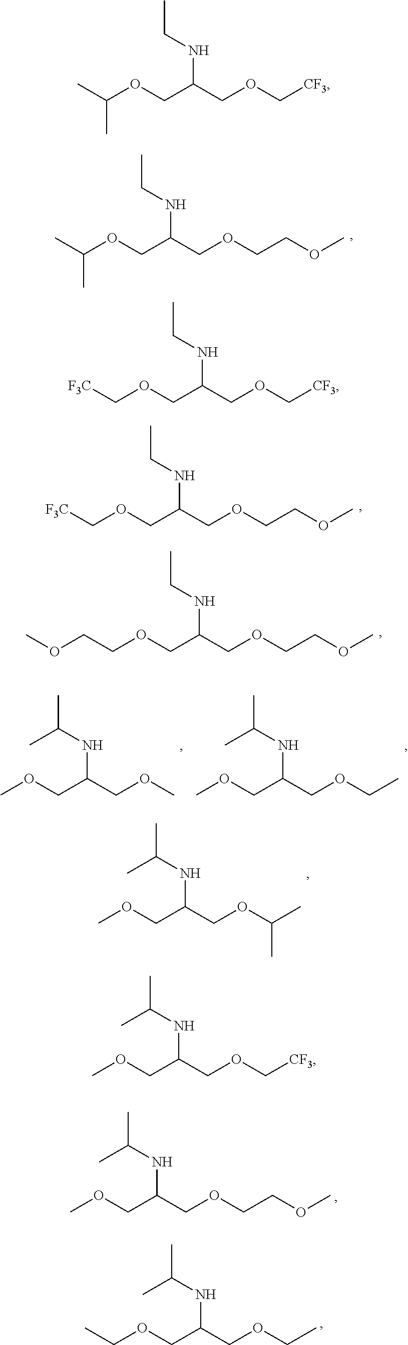

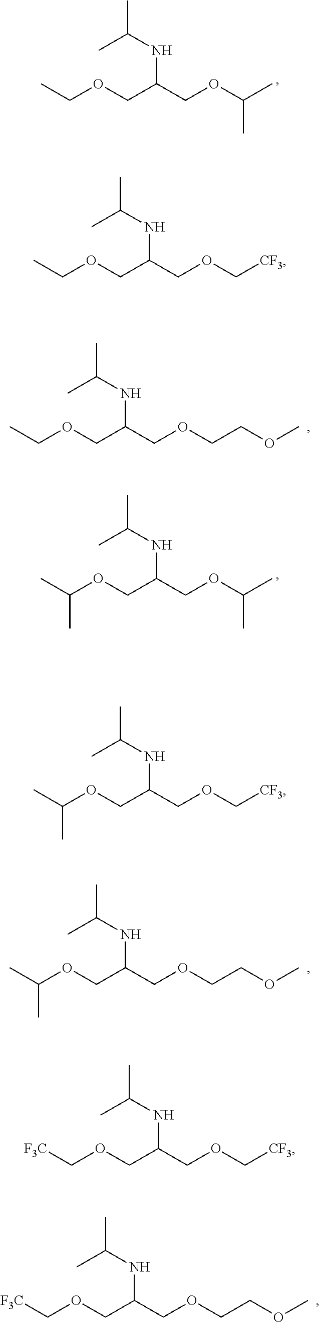

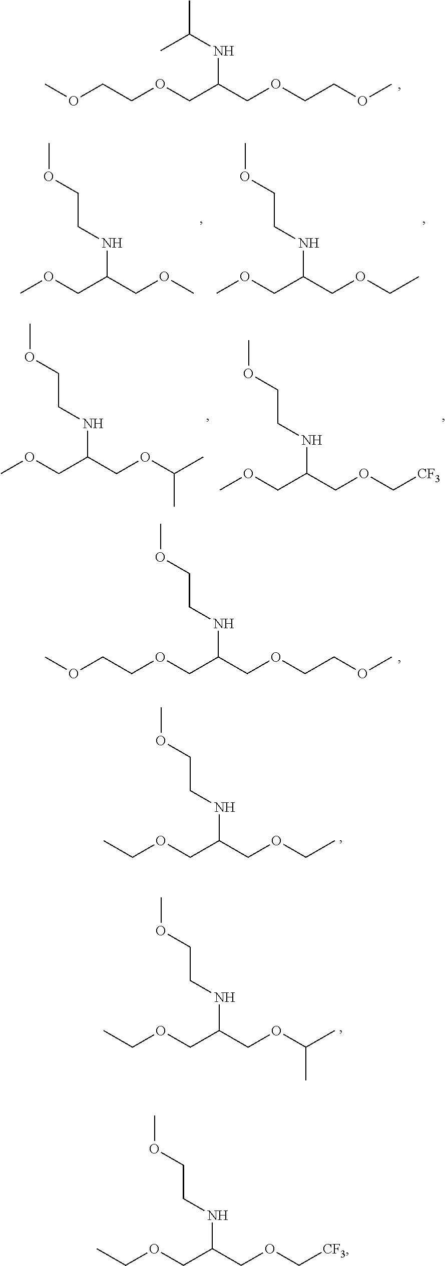

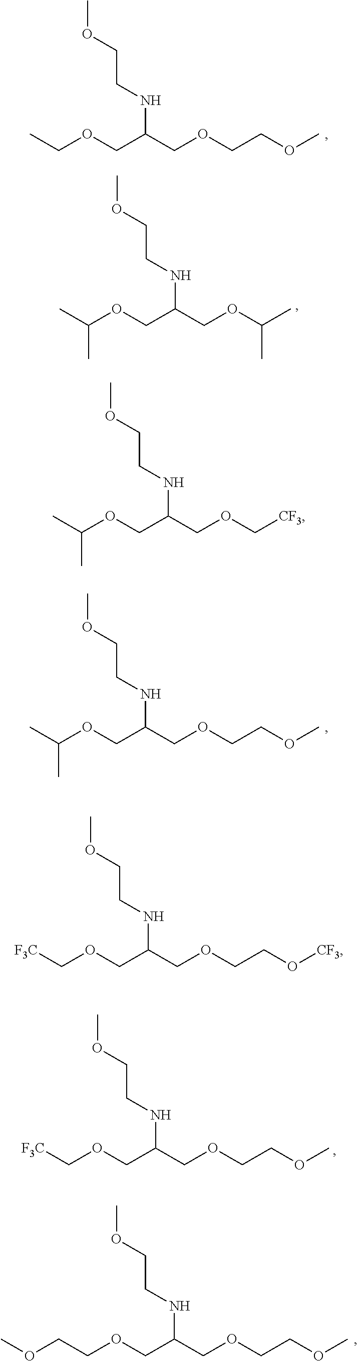

and combinations thereof.

Example 11: The compound of any examples herein, particularly examples 1-10, wherein the compound is selected from the group consisting of:

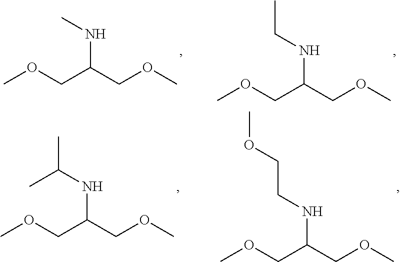

and combinations thereof.

Example 12: The compound of any examples herein, particularly examples 1-5, wherein R³ and R⁴ are each hydrogen.

Example 13: The compound of any examples herein, particularly examples 1-5 or 12, wherein the compound is defined by Formula III:

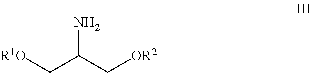

wherein R¹ and R² are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy; with the proviso that at least one of R¹-R² is not H.

Example 14: The compound of any examples herein, particularly examples 1-5 or 12-13, wherein the compound is selected from the group consisting of:

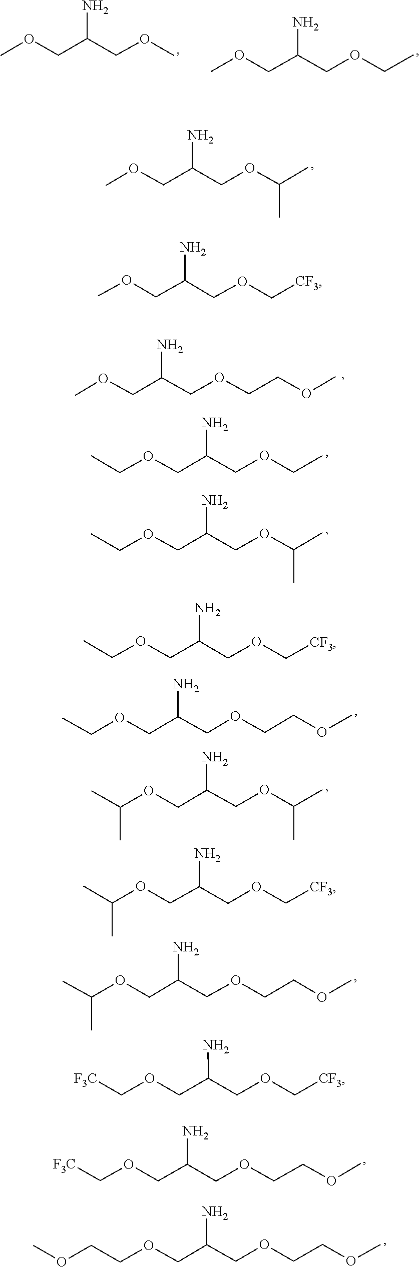

and combinations thereof.

Example 15: The compound of any examples herein, particularly examples 1-5 or 12-14, wherein the compound is selected from the group consisting of:

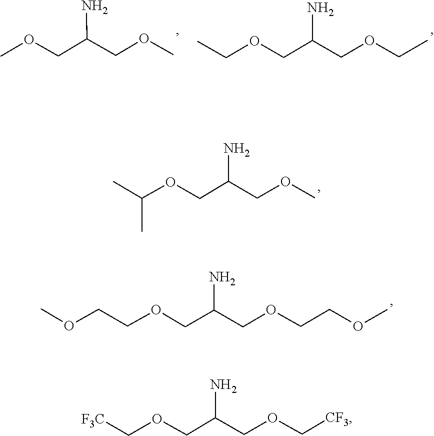

and combinations thereof.

Example 16: The compound of any examples herein, particularly examples 1-6, wherein R³ and R⁴ are each independently substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy, or wherein, as valence permits, R³ and R⁴, together with the atoms to which they are attached, form a 3-10 membered substituted or unsubstituted cyclic moiety, optionally including 1 to 3 additional heteroatoms.

Example 17: The compound of any examples herein, particularly examples 1-6 or 16, wherein R³ and R⁴ are each independently substituted or unsubstituted C₁₋₂₀ alkyl, or substituted or unsubstituted C₁-C₂₀ alkoxy, or wherein, as valence permits, R³ and R⁴, together with the atoms to which they are attached, form a 3-10 membered substituted or unsubstituted cyclic moiety, optionally including 1 to 3 additional heteroatoms.

Example 18: The compound of any examples herein, particularly examples 1-6 or 16-17, wherein R³ and R⁴ are each independently substituted or unsubstituted C₁₋₆ alkyl, or substituted or unsubstituted C₁-C₆ alkoxy, or wherein, as valence permits, R³ and R⁴, together with the atoms to which they are attached, form a 6 membered substituted or unsubstituted cyclic moiety, optionally including 1 additional heteroatom.

Example 19: The compound of any examples herein, particularly examples 1-6 or 16-18, wherein R³ and R⁴ are each independently methyl, ethyl, isopropyl, or —CH₂CH₂OCH₃, or R³ and R⁴, together with the atoms to which they are attached, form a piperazine.

Example 20: The compound of any examples herein, particularly examples 1-6 or 16-19, wherein R³ and R⁴ are each independently methyl, ethyl, isopropyl, or —CH₂CH₂OCH₃.

Example 21: The compound of any examples herein, particularly examples 1-6 or 16-19, wherein R³ and R⁴ together with the atoms to which they are attached, form a piperazine.

Example 22: The compound of any examples herein, particularly examples 1-6 or 16-21, wherein the compound is selected from the group consisting of:

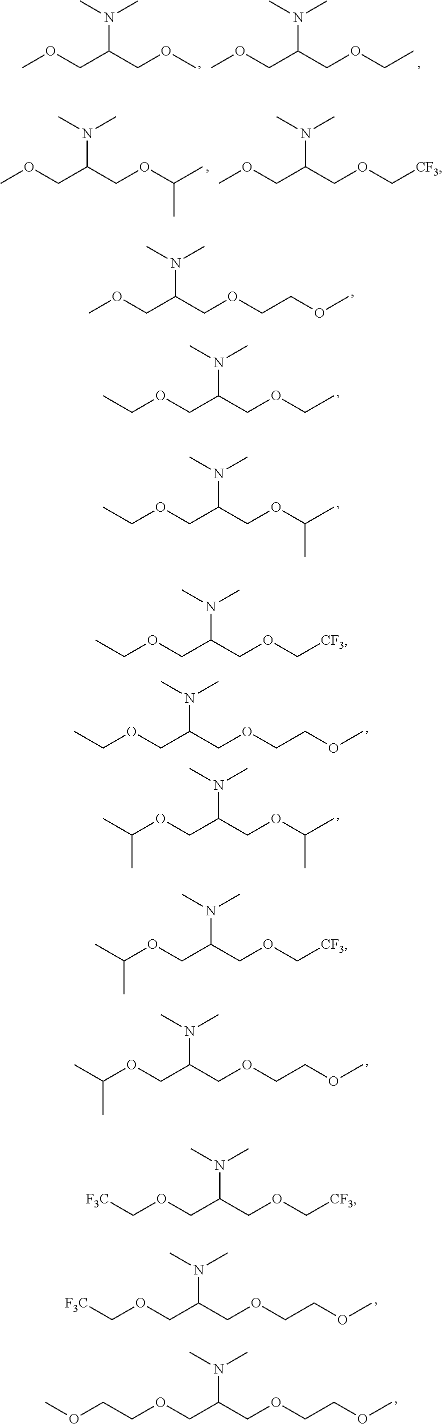

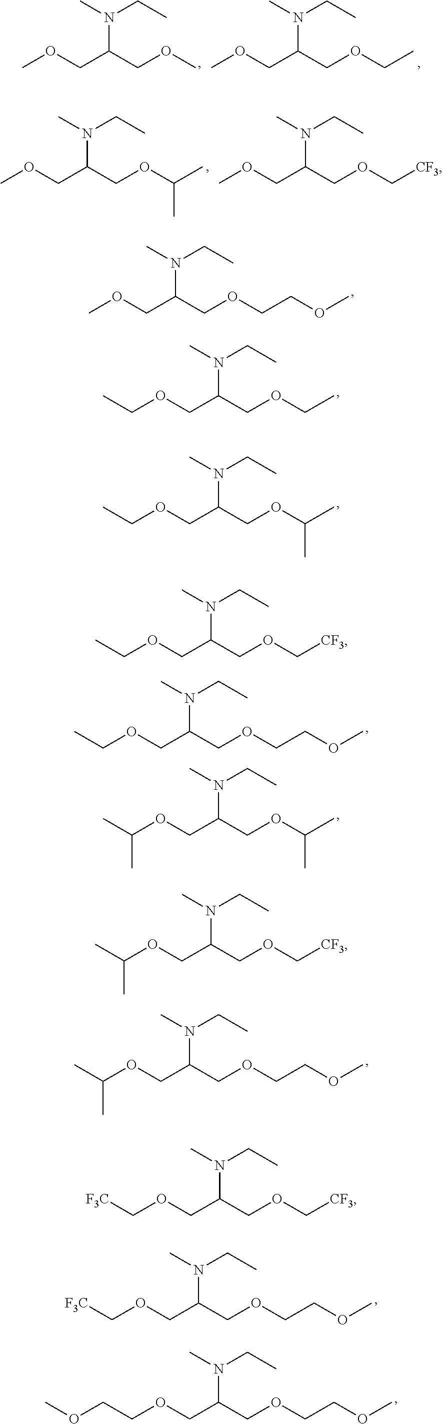

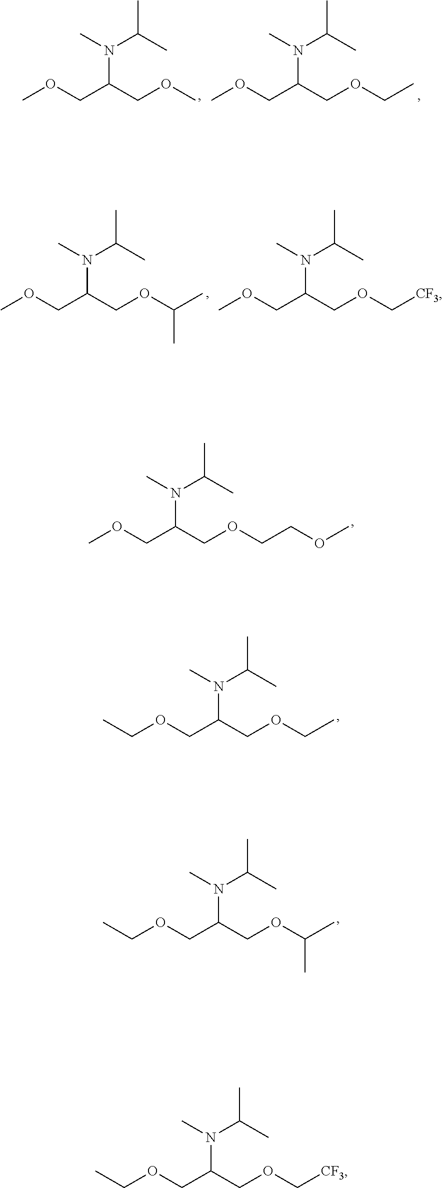

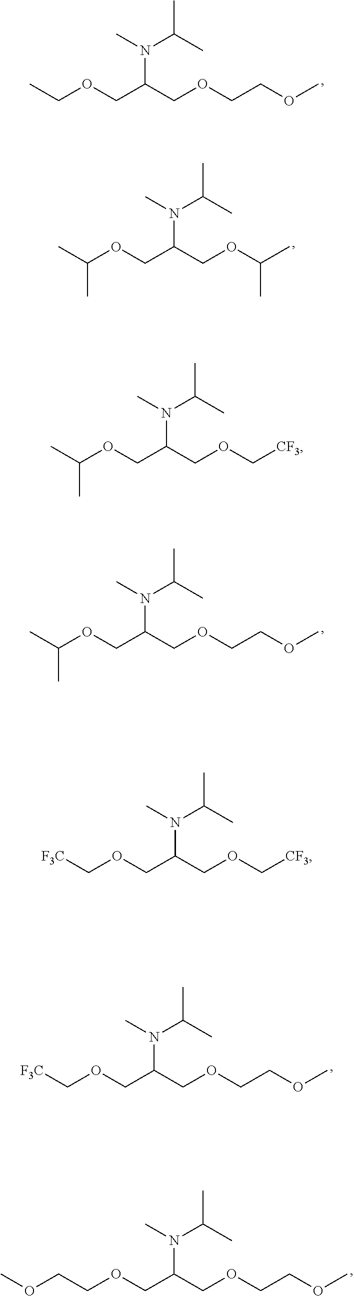

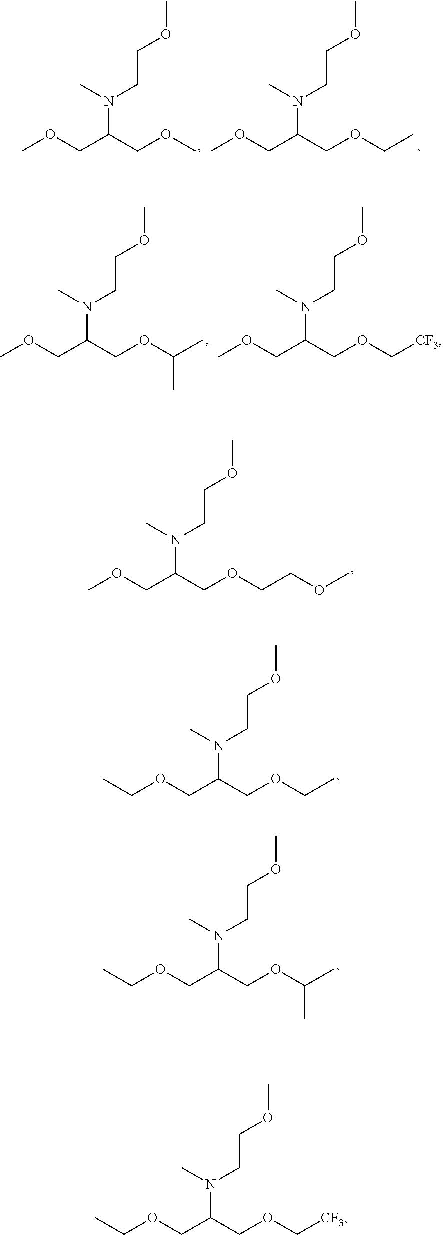

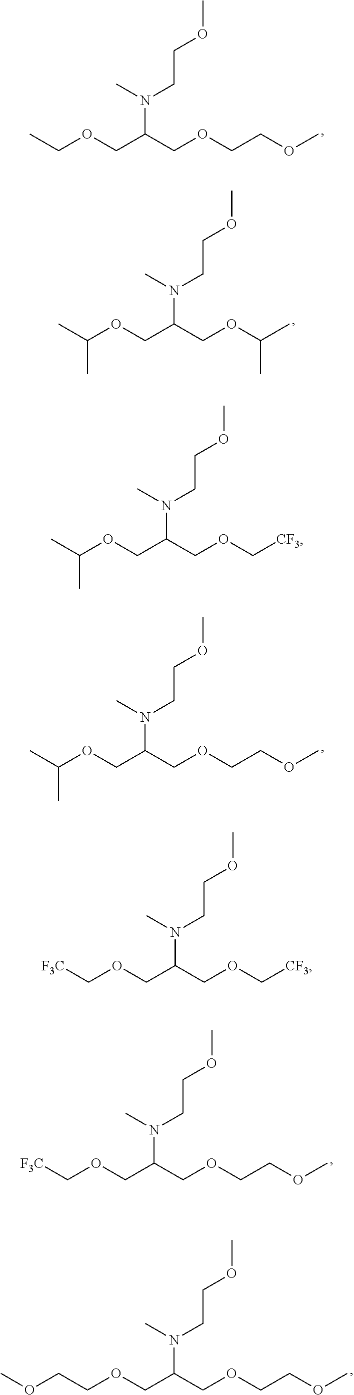

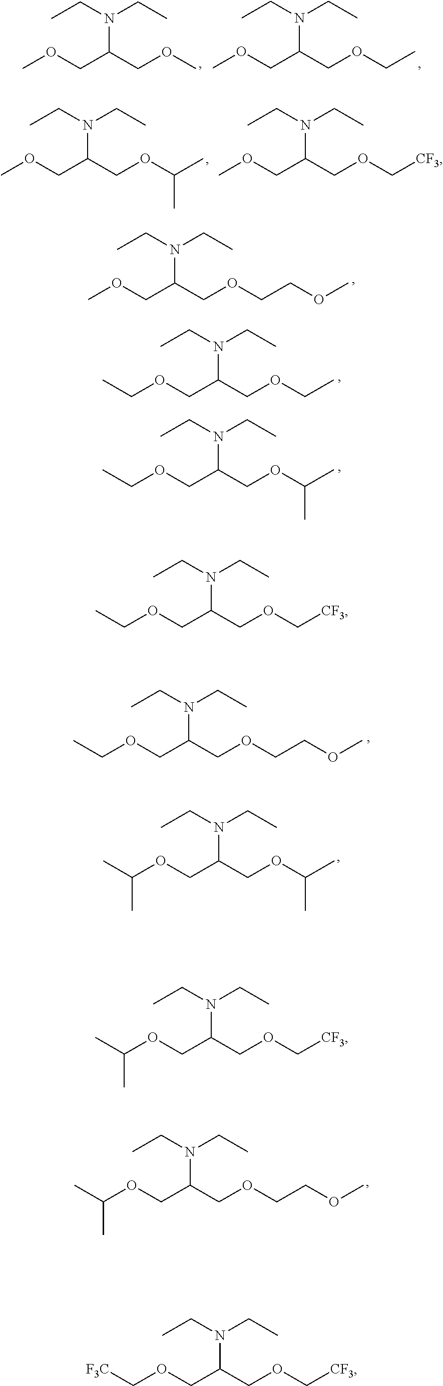

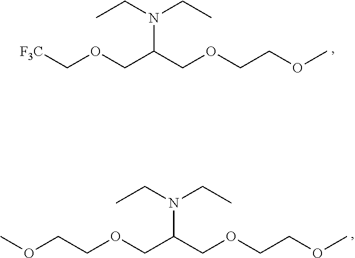

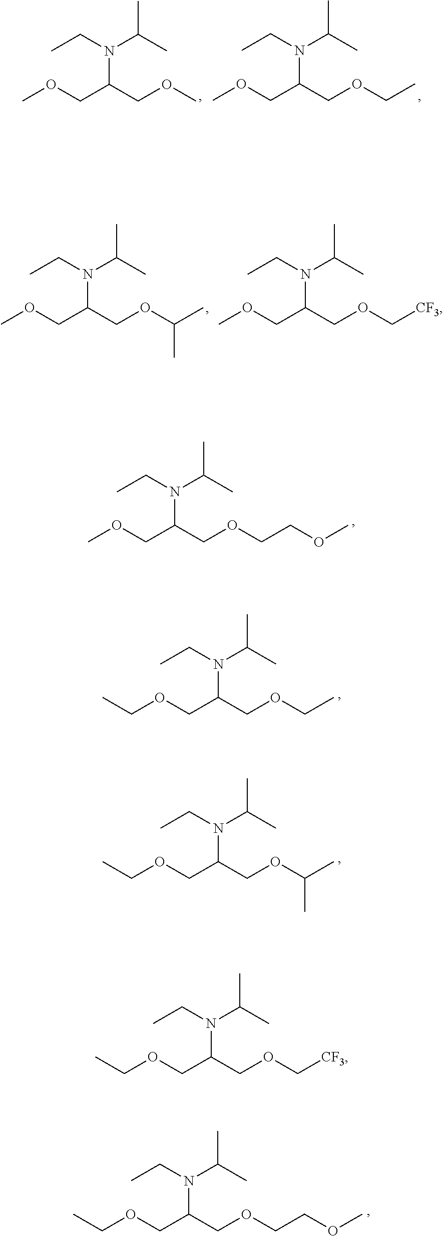

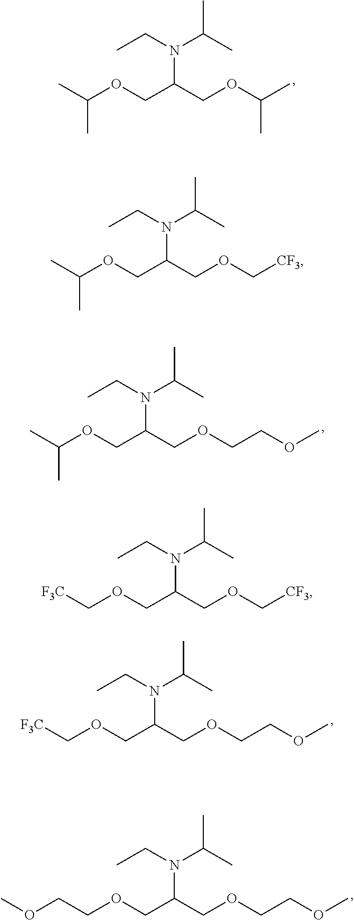

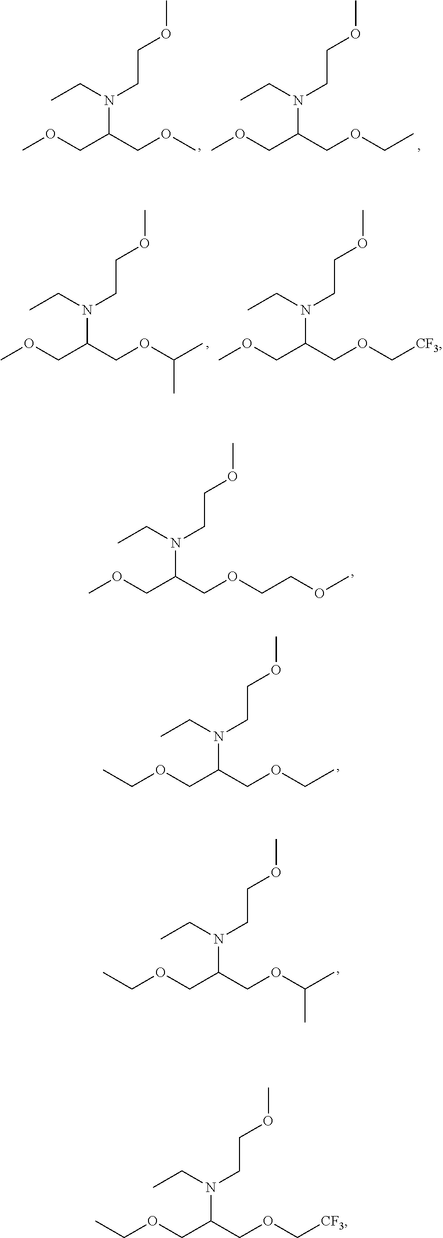

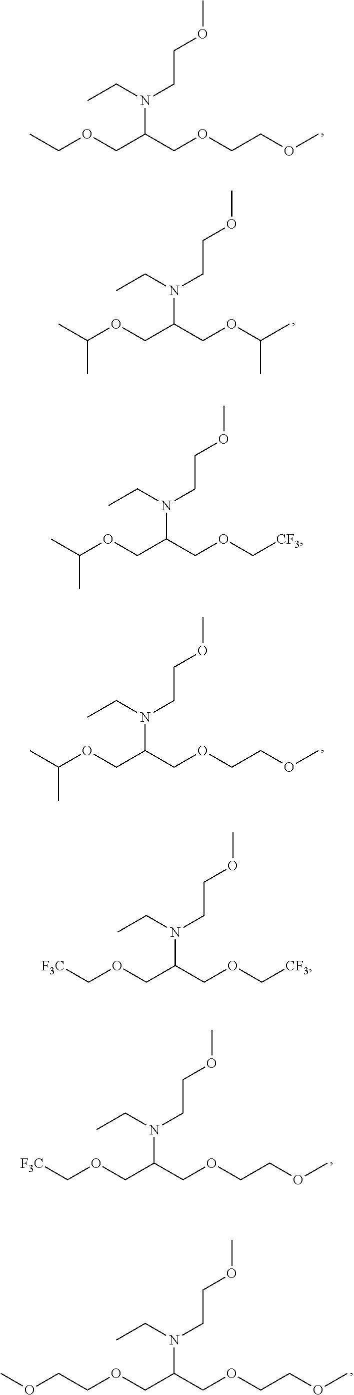

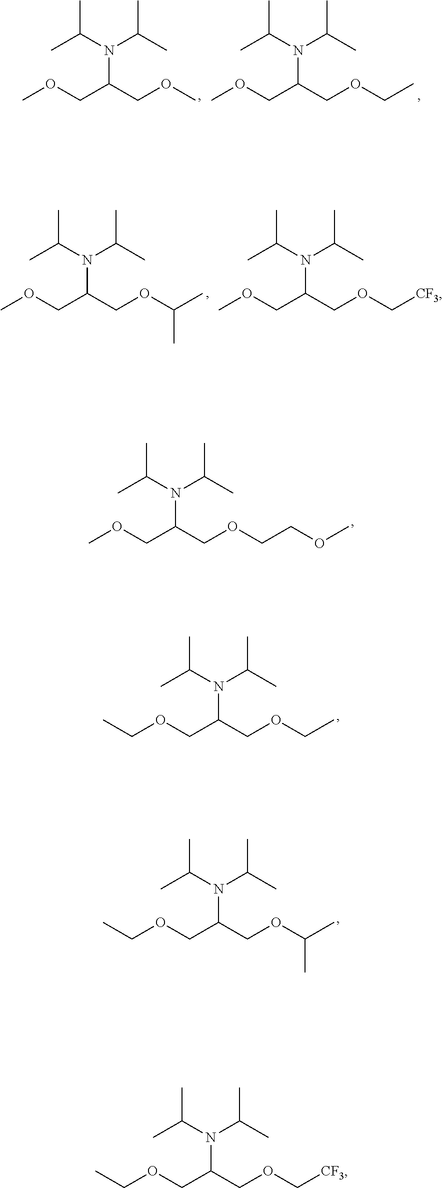

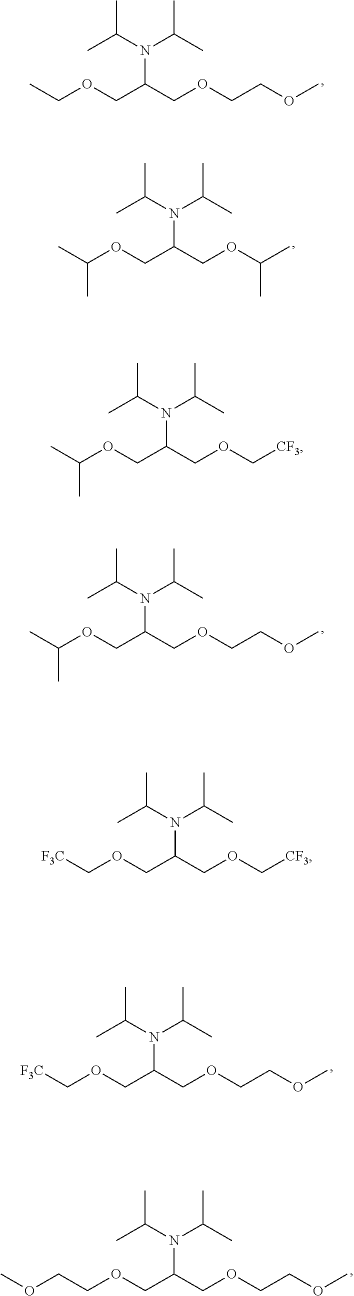

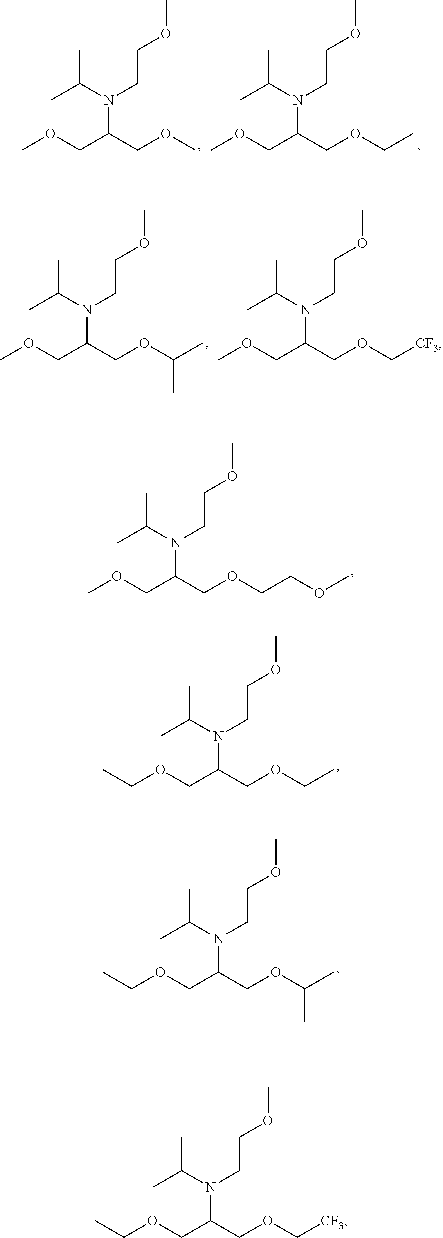

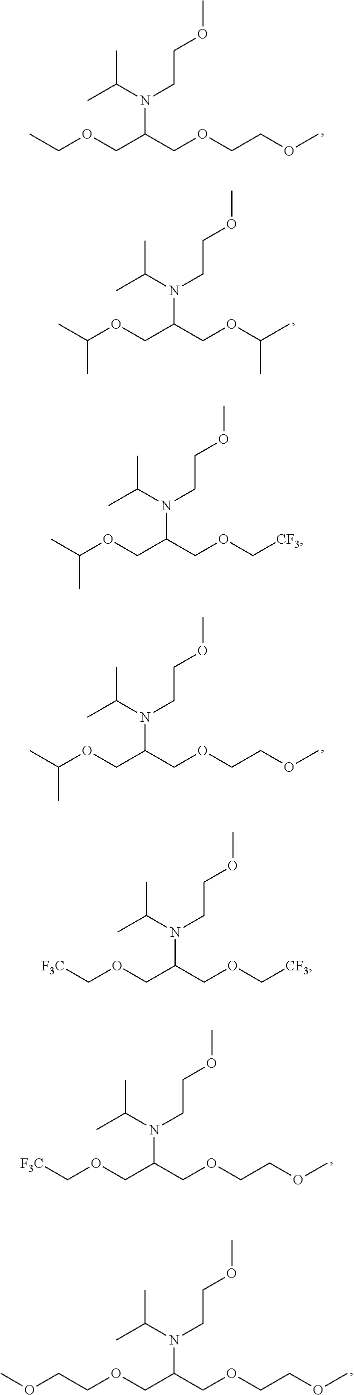

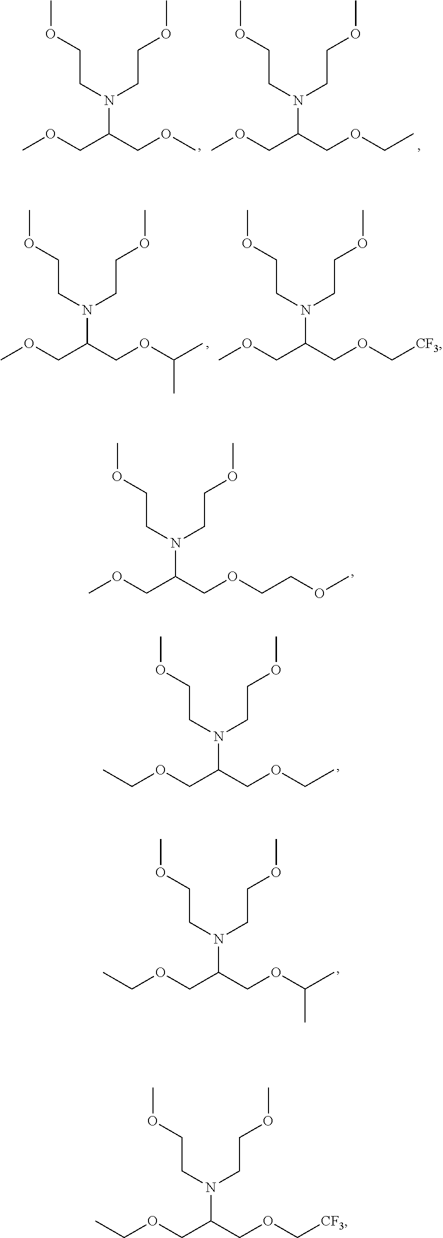

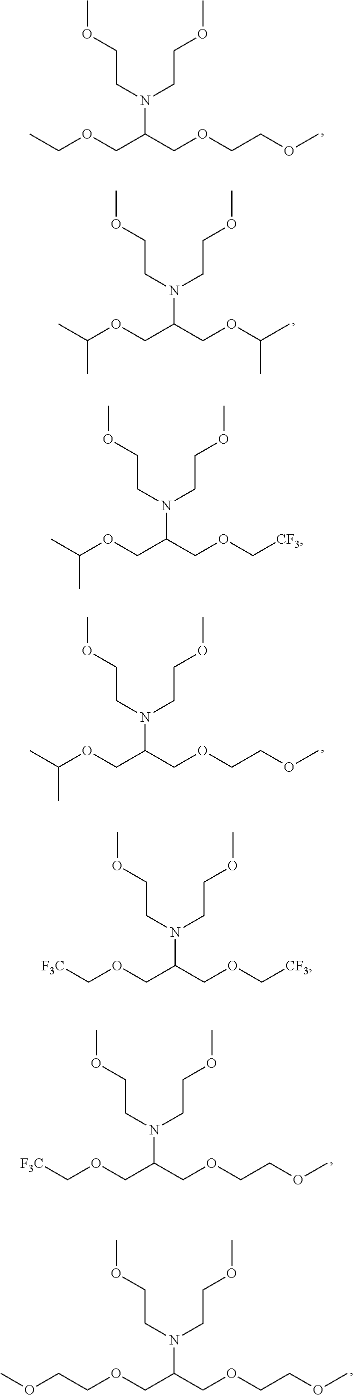

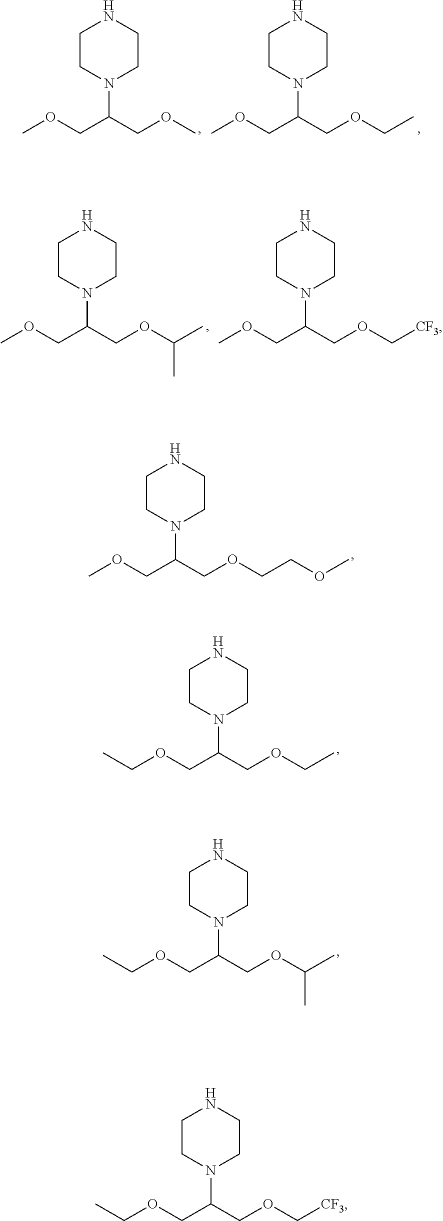

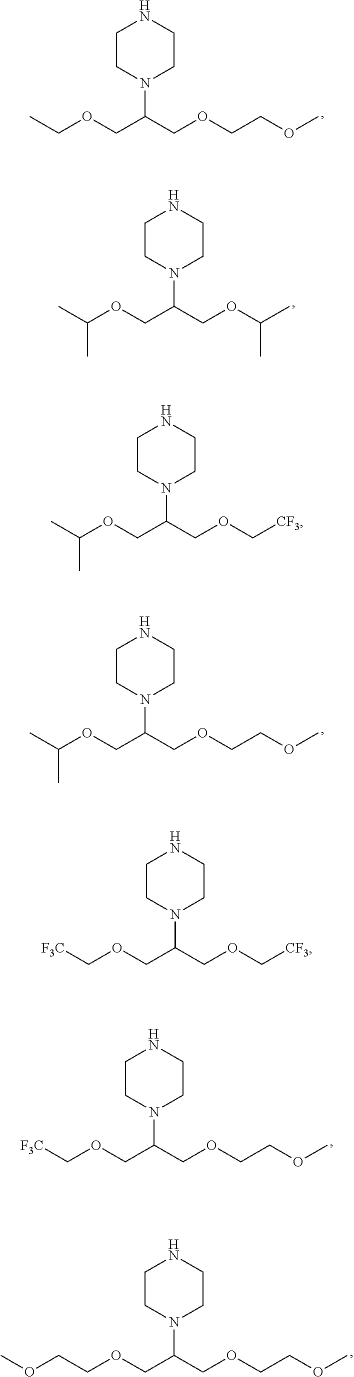

and combinations thereof.

Example 23: The compound of any examples herein, particularly examples 1-6 or 16-22, wherein the compound is selected from the group consisting of:

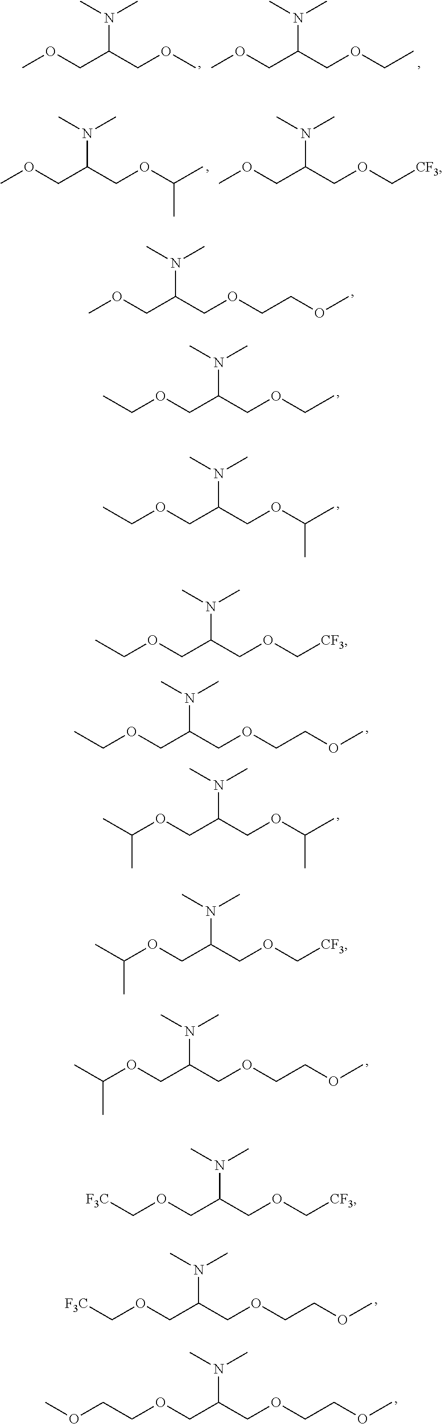

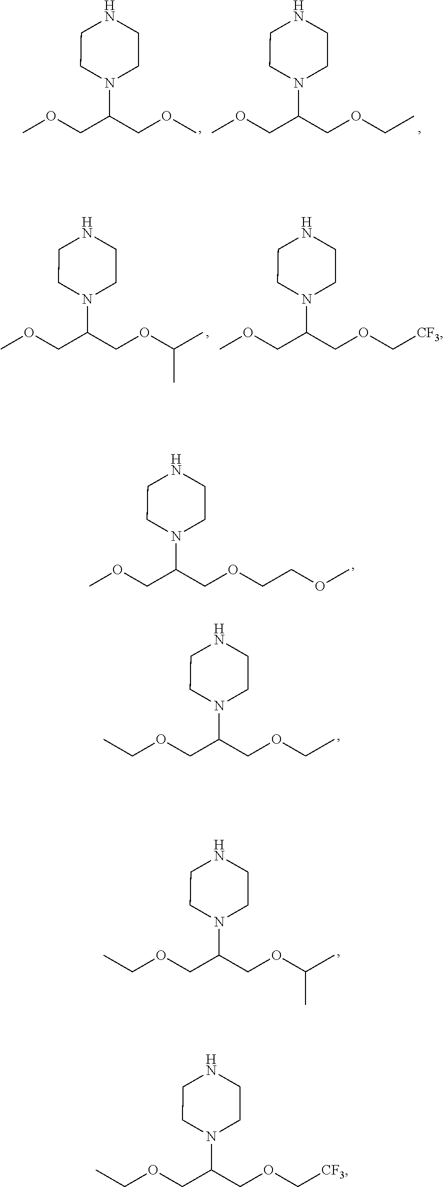

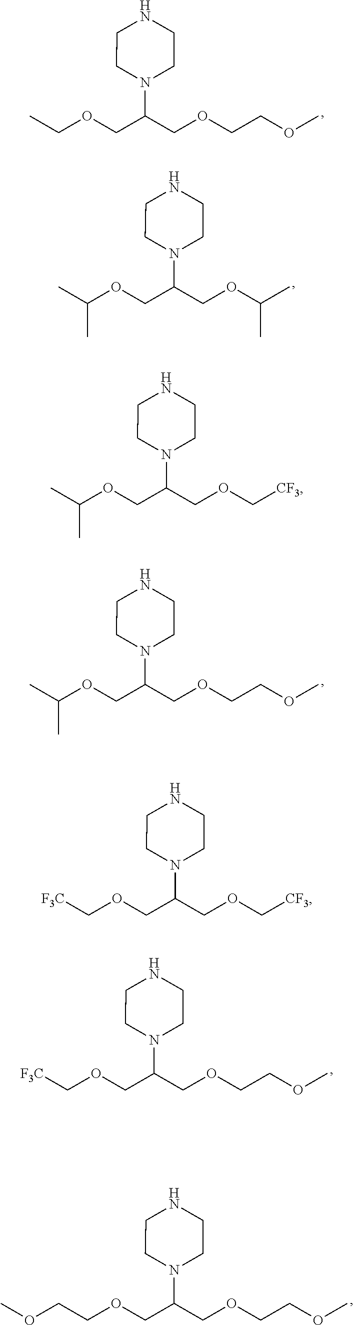

and combinations thereof.

Example 24: The compound of any examples herein, particularly examples 1-6 or 16-23, wherein the compound is selected from the group consisting of:

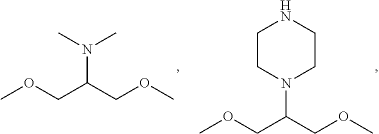

and combinations thereof.

Example 25: The compound of any examples herein, particularly examples 1-24, wherein R¹ and R² are the same.

Example 26: The compound of any examples herein, particularly examples 1-25, wherein R¹ and R² are different.

Example 27: The compound of any examples herein, particularly examples 1-26, wherein R¹ and R² are each independently substituted or unsubstituted C₁₋₂₀ alkyl, or substituted or unsubstituted C₁-C₂₀ alkoxy.

Example 28: The compound of any examples herein, particularly examples 1-27, wherein R¹ and R² are each independently substituted or unsubstituted C₁₋₆ alkyl, or substituted or unsubstituted C₁-C₆ alkoxy.

Example 29: The compound of any examples herein, particularly examples 1-28, wherein R¹ and R² are each independently C₁₋₆ alkyl optionally substituted with one or more substituents selected from the group consisting of ether, C₁-C₅ alkyl, halogen, amine, and C₁-C₅ alkoxy.

Example 30: The compound of any examples herein, particularly examples 1-29, wherein R¹ and R² are each independently C₁₋₆ alkyl optionally substituted with halogen (e.g., C₁-C₆ fluoroalkyl, such as trifluoromethyl terminated alkyl).

Example 31: The compound of any examples herein, particularly examples 1-30, wherein R¹ and R² are each independently methyl, ethyl, isopropyl, —CH₂CF₃, —CH₂CH₂OCH₃.

Example 32: The compound of any examples herein, particularly examples 1-31, wherein the compound is selected from the group consisting of:

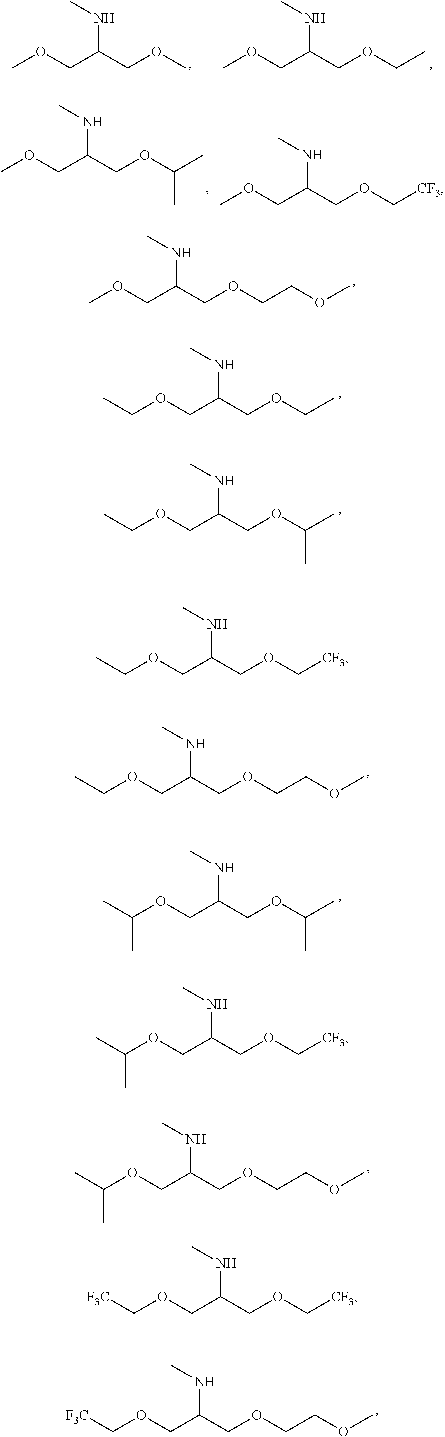

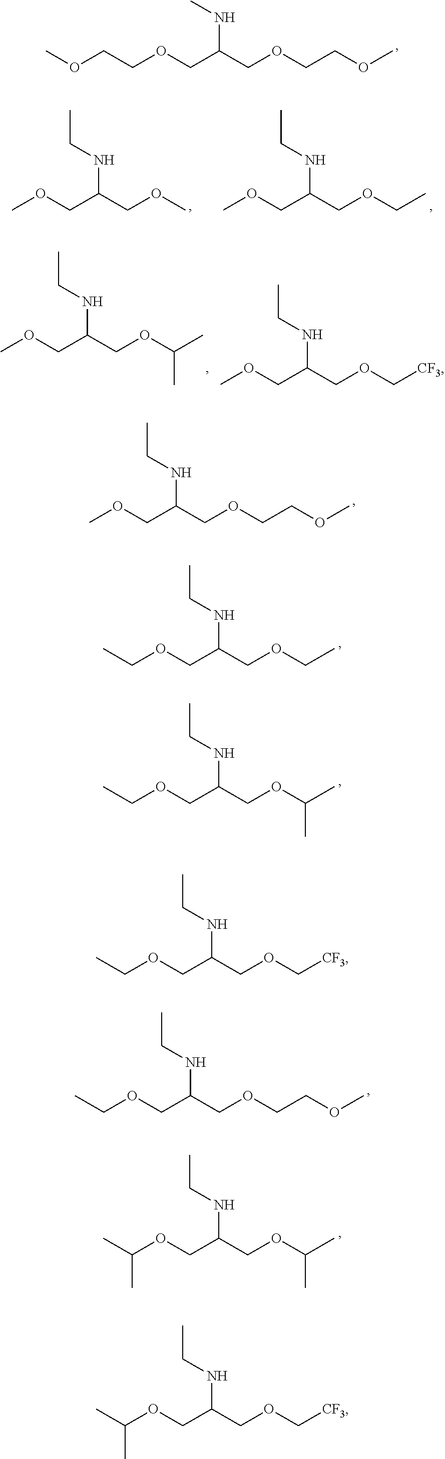

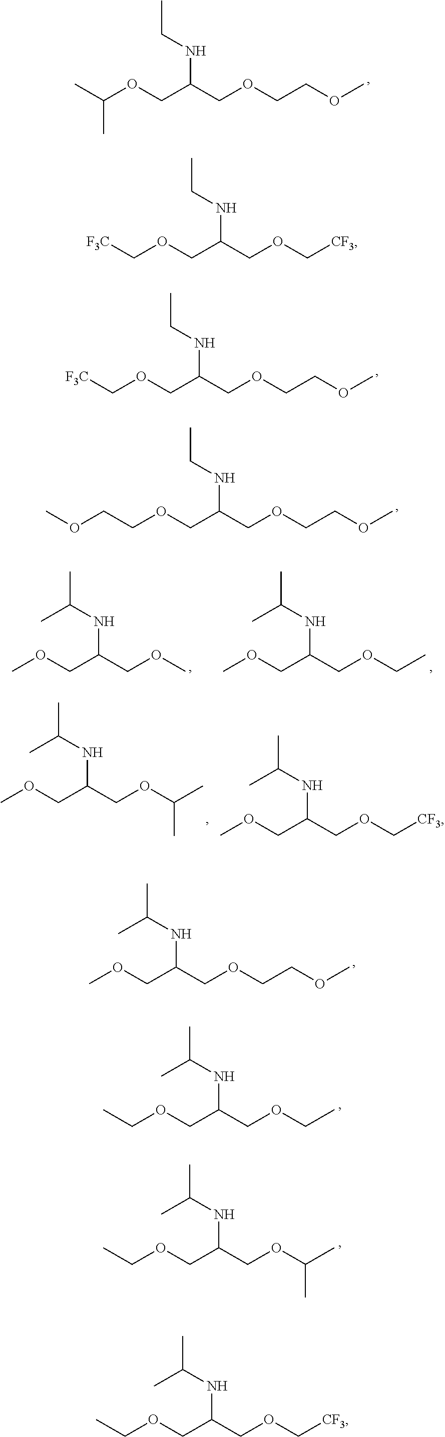

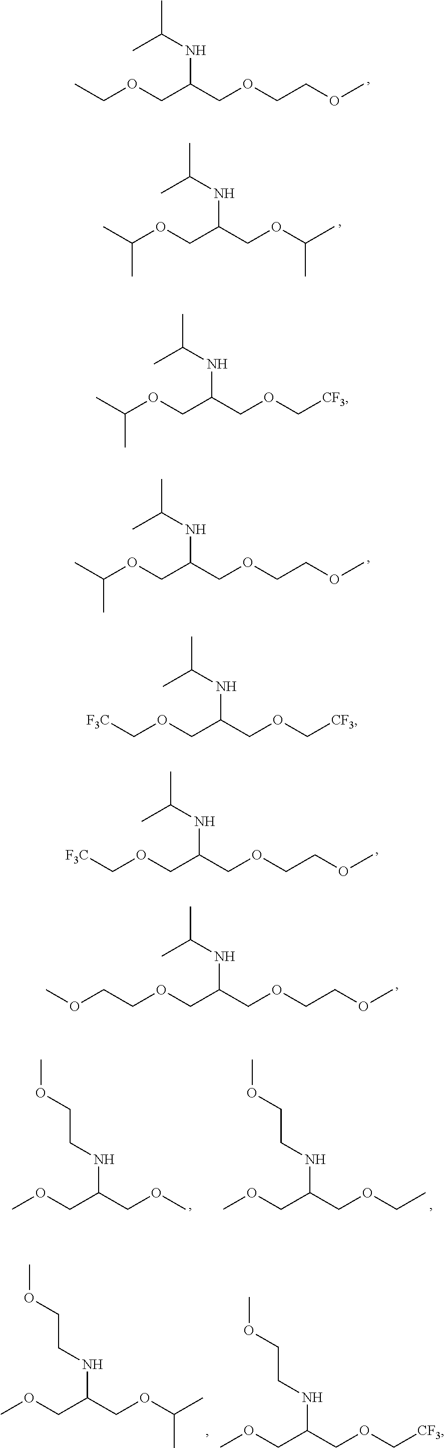

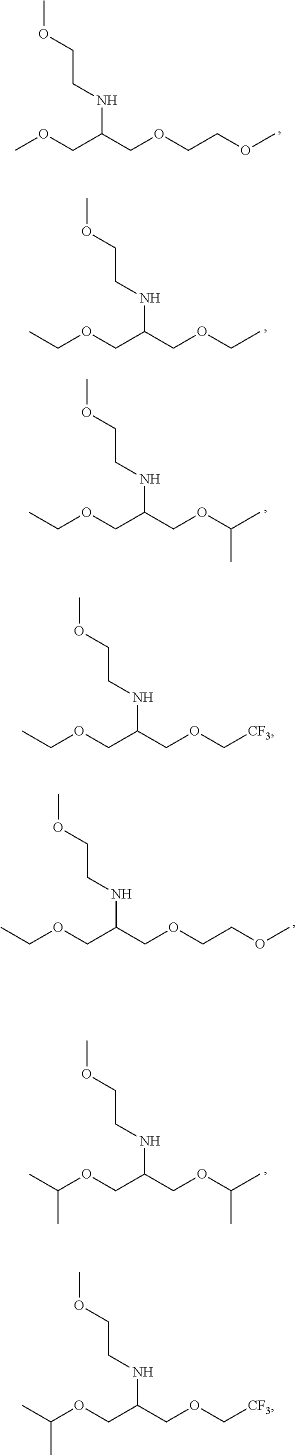

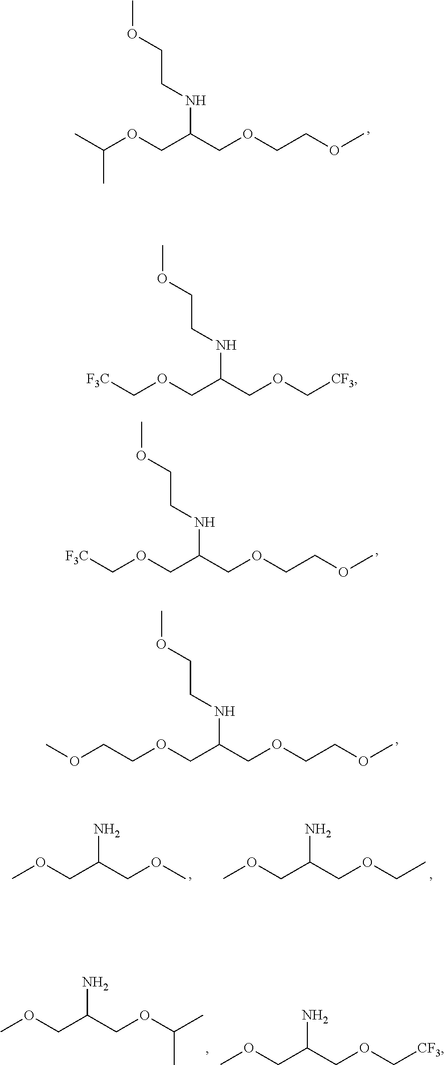

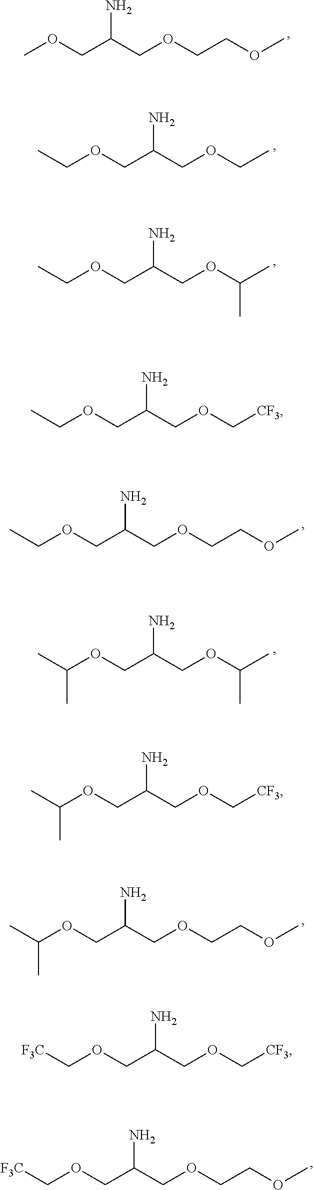

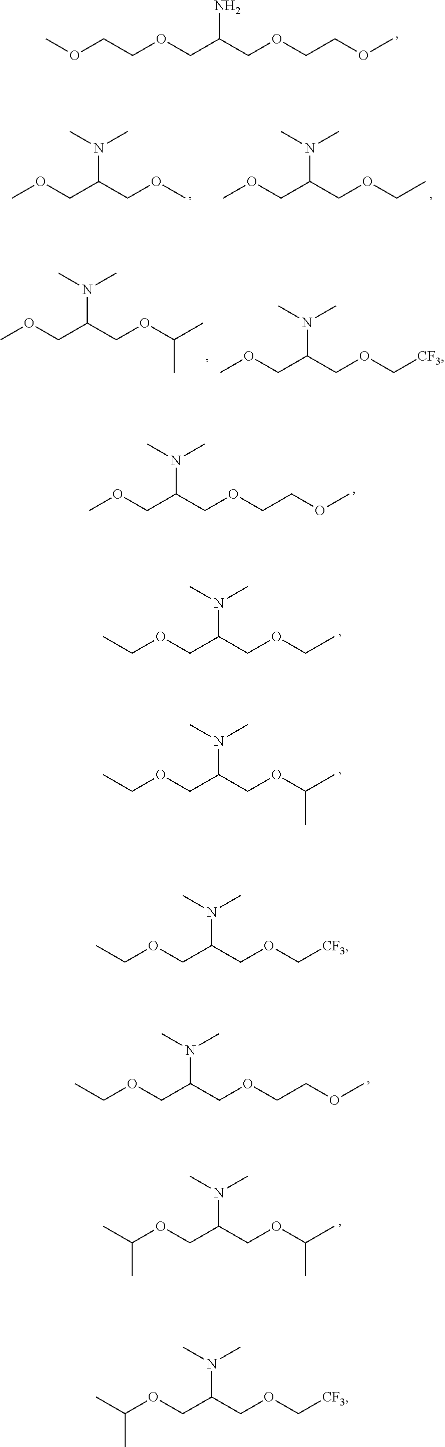

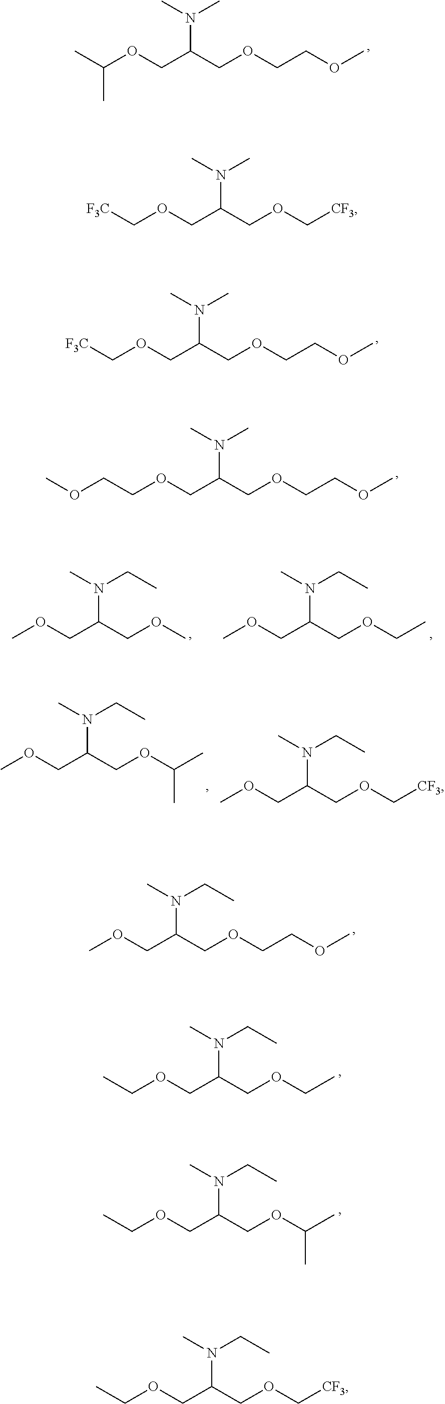

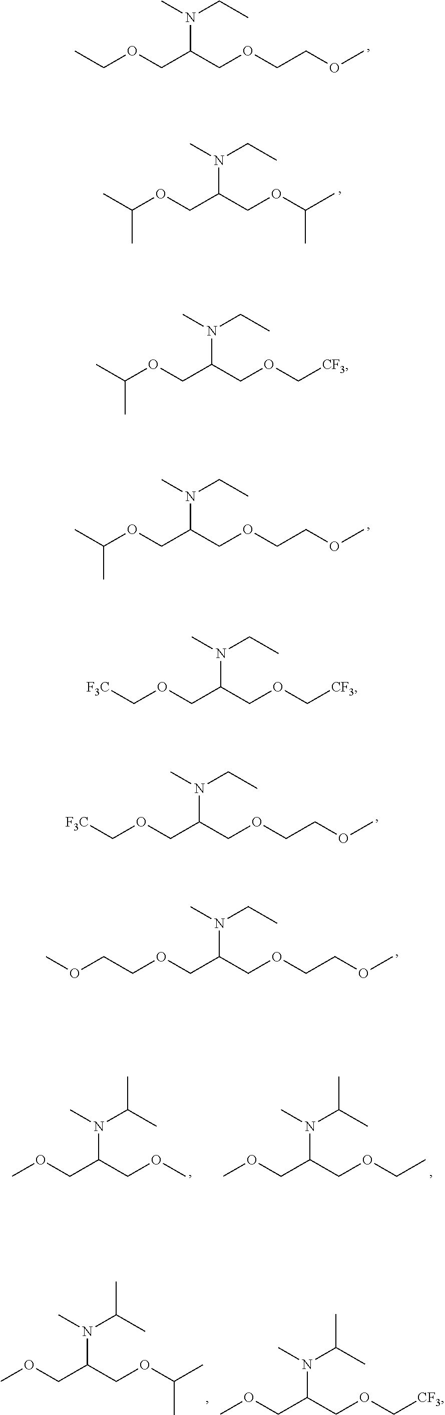

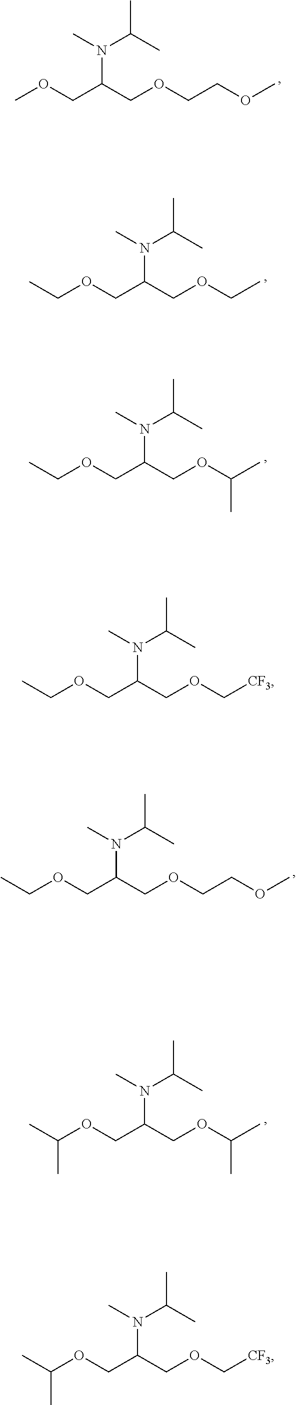

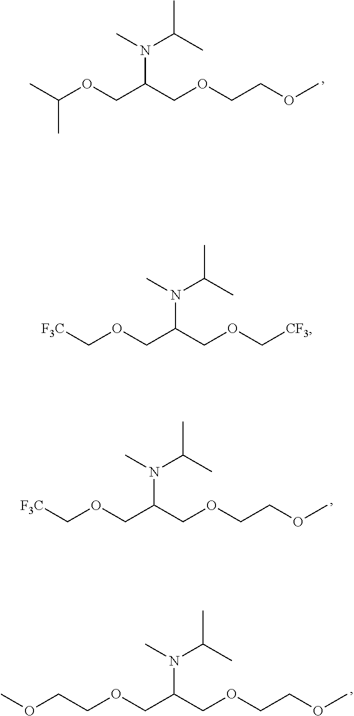

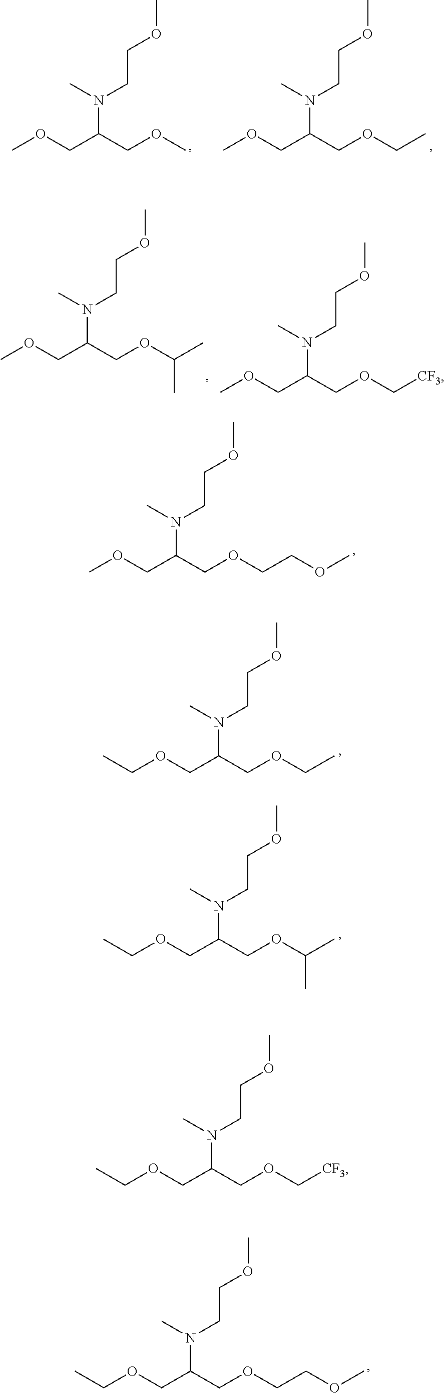

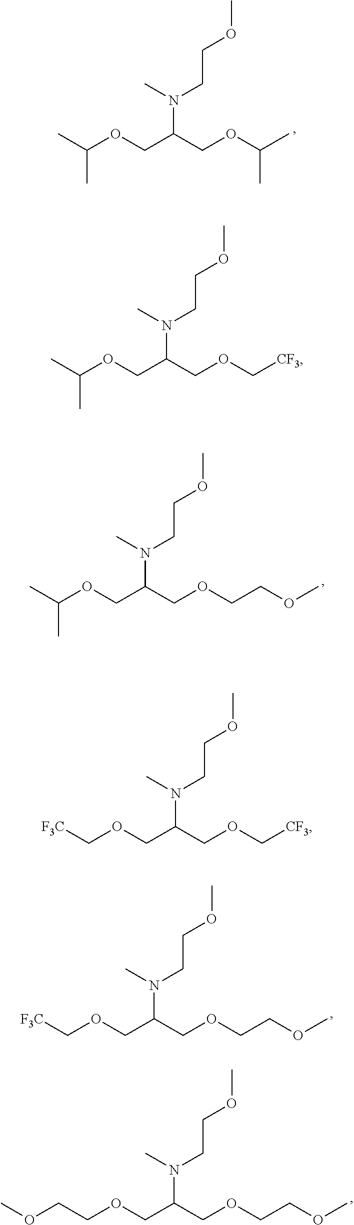

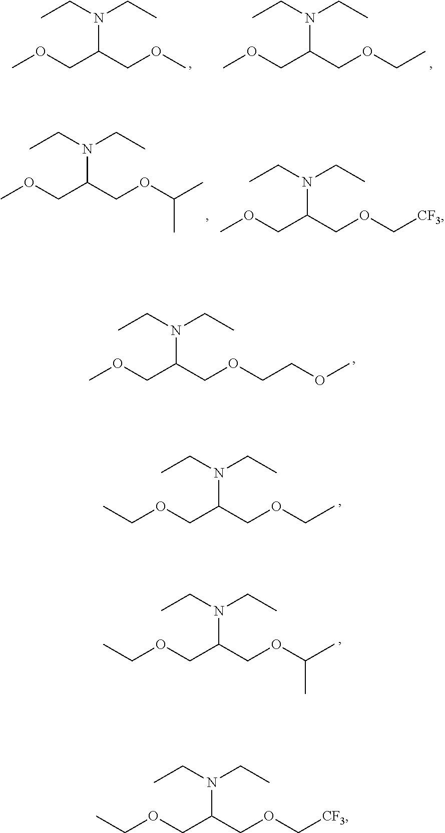

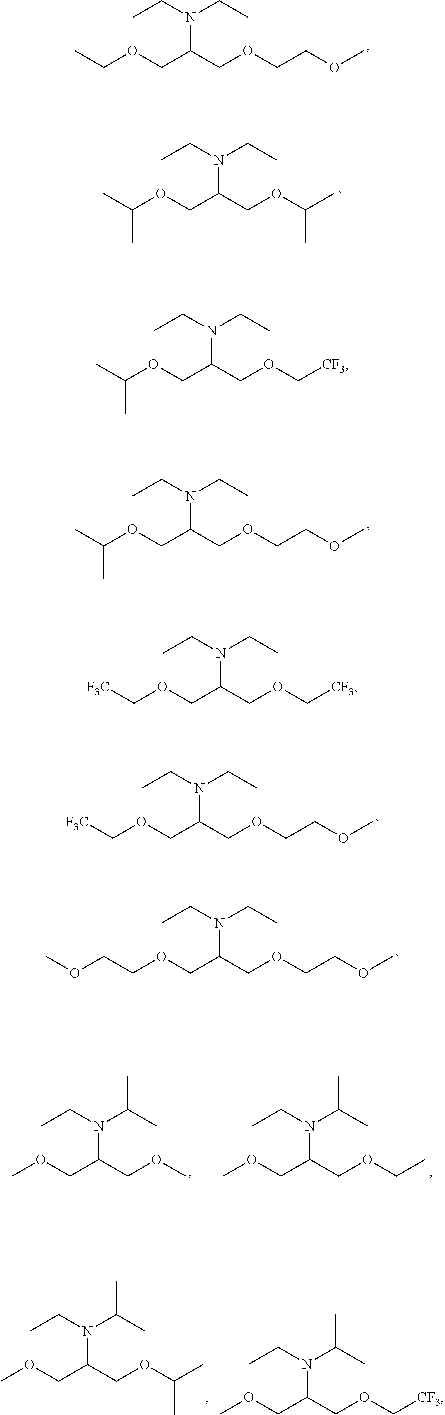

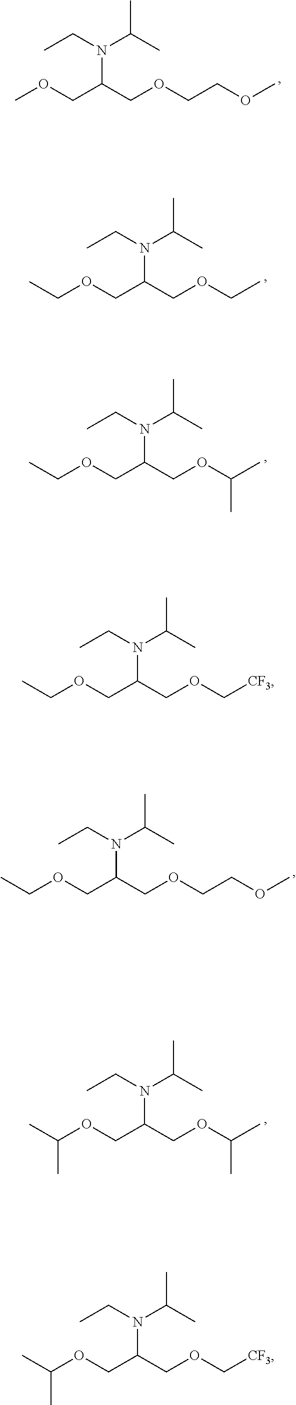

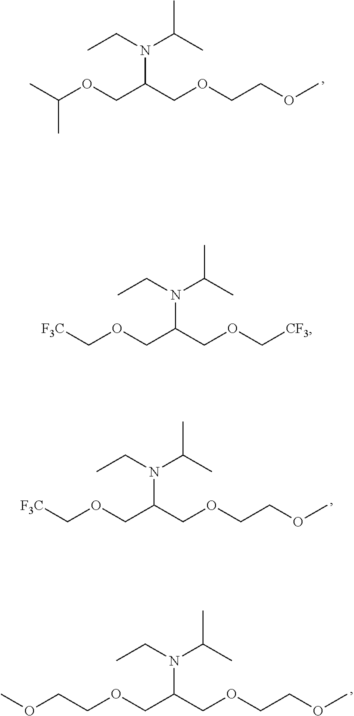

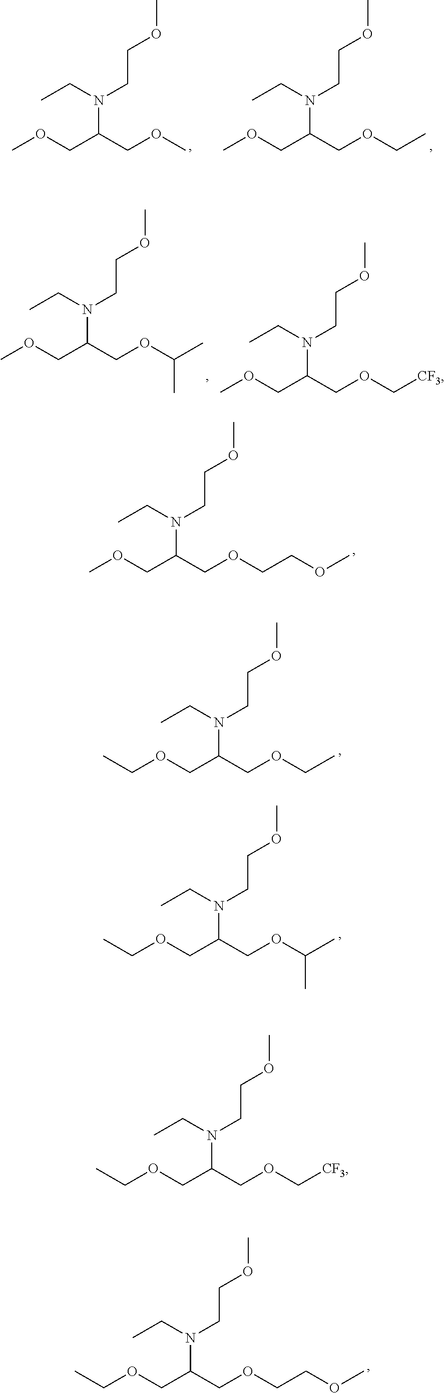

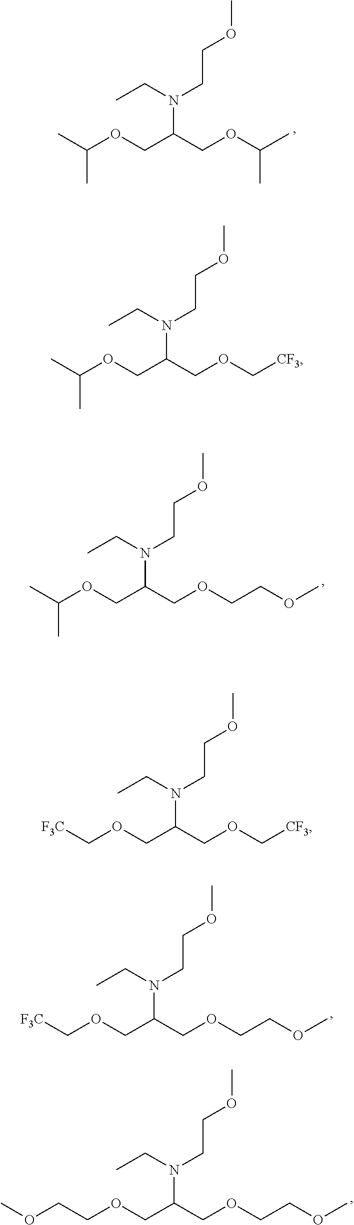

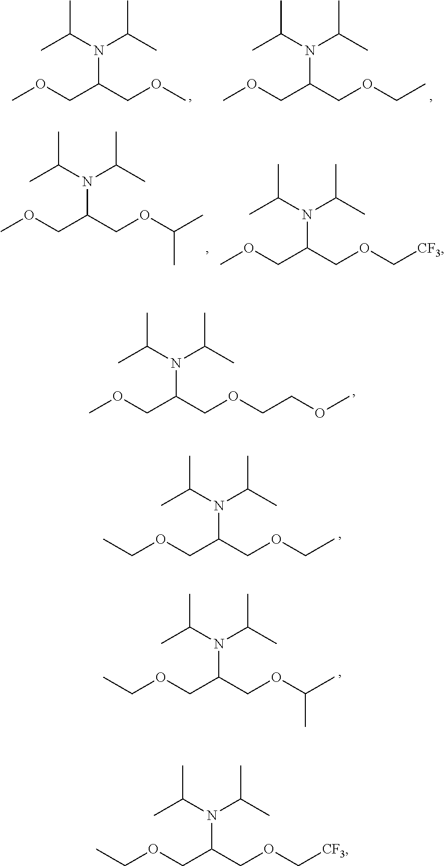

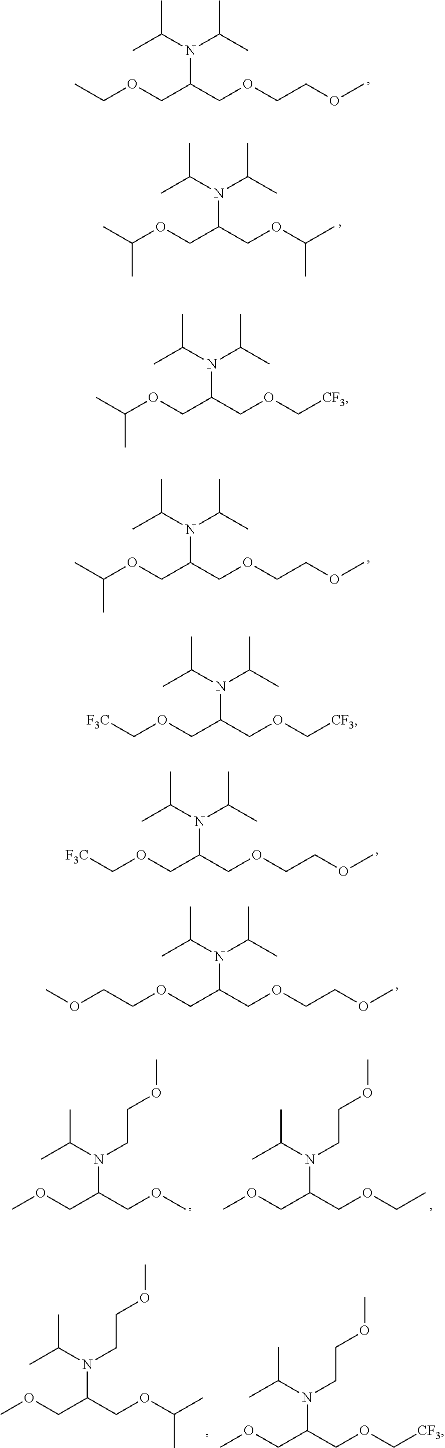

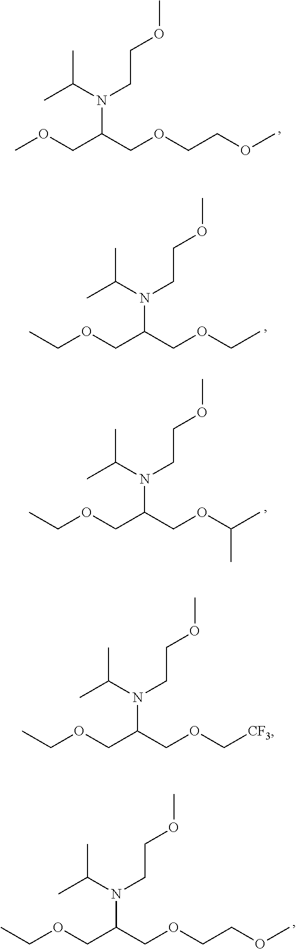

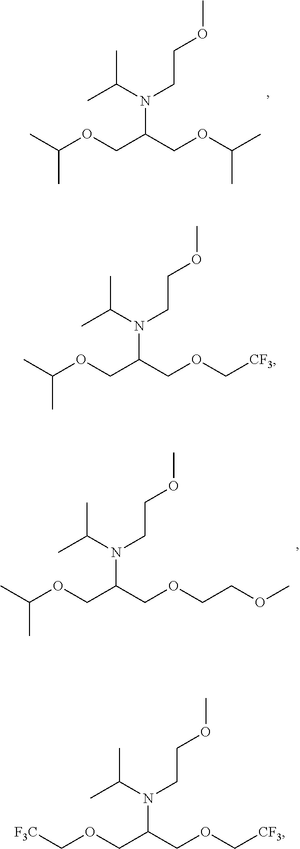

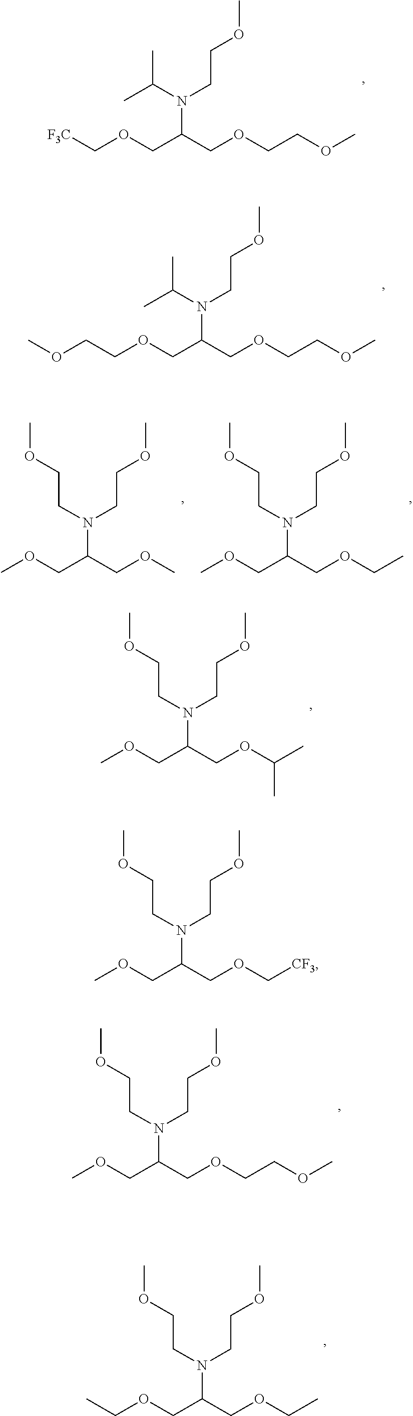

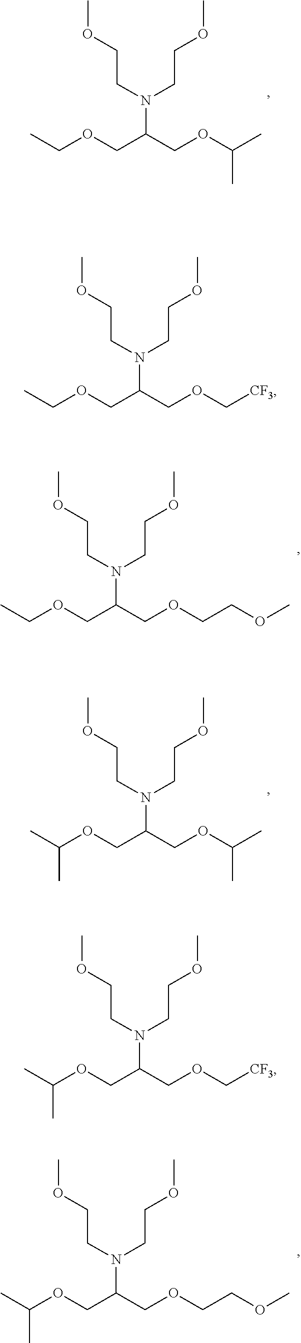

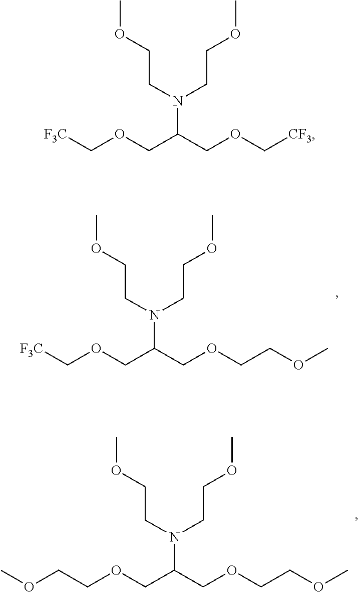

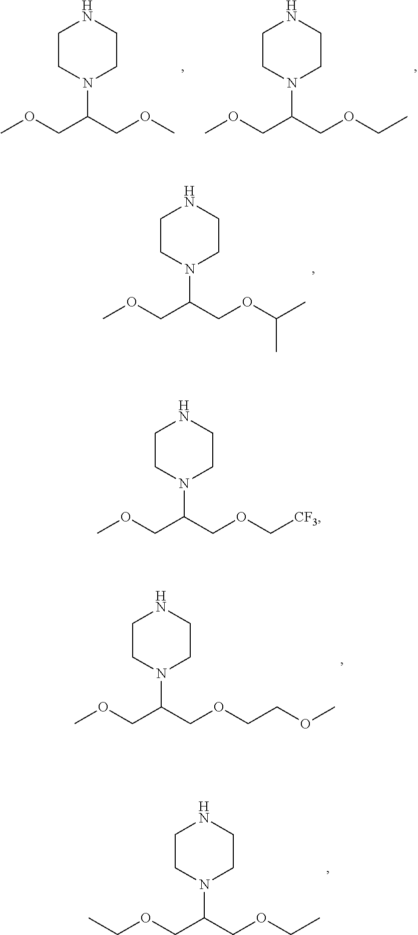

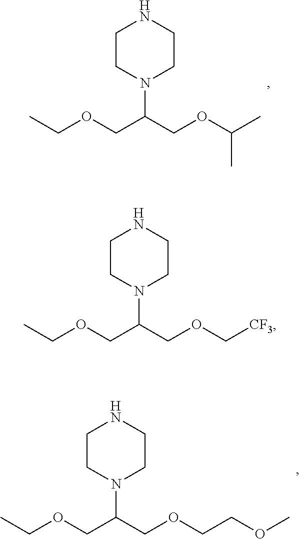

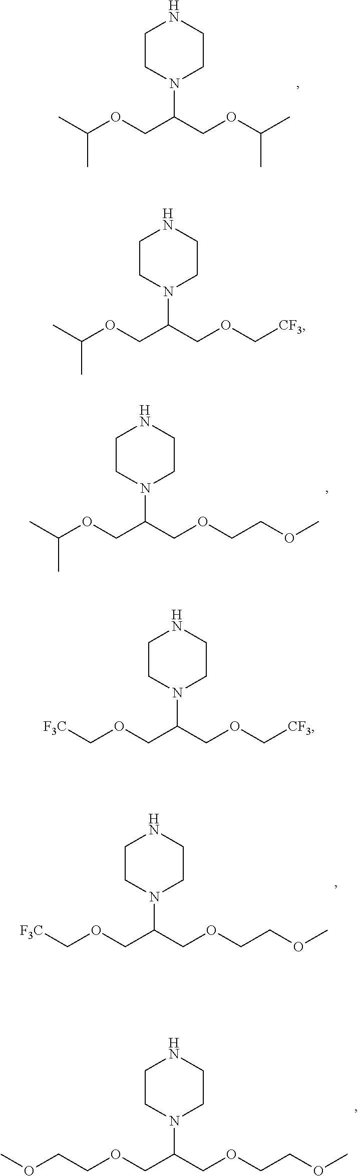

and combinations thereof.

Example 33: The compound of any examples herein, particularly examples 1-32, wherein the compound is selected from the group consisting of:

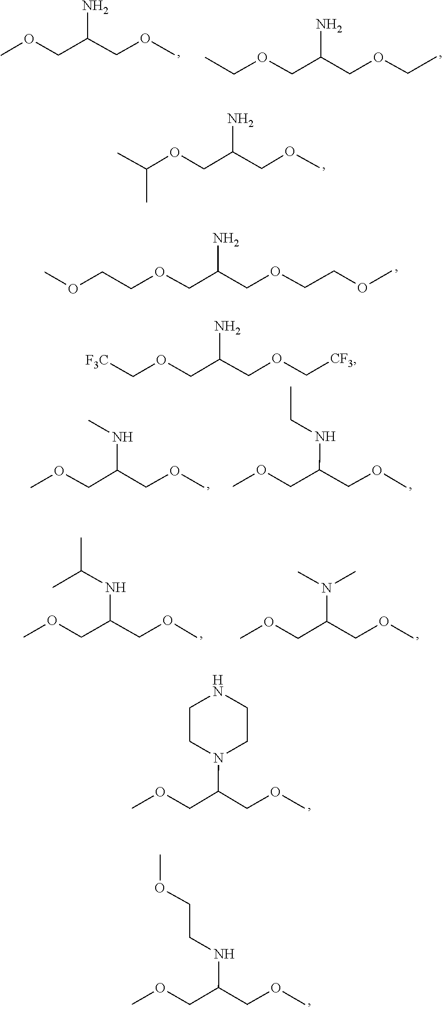

and combinations thereof.

Example 34: A solvent system for capturing an acid gas from a fluid stream, wherein the solvent system comprises the compound of any examples herein, particularly examples 1-33.

Example 35: The solvent system of any examples herein, particularly example 34, wherein the solvent system is water-lean.

Example 36: The solvent system of any examples herein, particularly example 34, wherein the solvent system further comprises an additional solvent, such as water.

Example 37: A method of use of the compound of any examples herein, particularly examples 1-33 or the solvent system of any examples herein, particularly examples 36-38 for capturing an acid gas from a fluid steam, the method comprising contacting the compound or solvent system with the fluid stream, wherein the fluid stream comprises the acid gas, thereby capturing the acid gas from the fluid stream.

Example 38: The method of any examples herein, particularly example 37, wherein the acid gas is captured by the compound by reacting with the compound to form a product.

Example 39: The method of any examples herein, particularly example 37 or example 38, wherein the method further comprises regenerating the compound.

Example 40: The method of any examples herein, particularly example 39, wherein regenerating the compound comprises exposing the product to steam and/or vacuum to strip the acid gas from the compound.

Example 41: The method of any examples herein, particularly example 40, further comprising collecting the acid gas after it has been stripped from the compound.

Example 42: The method of any examples herein, particularly examples 37-41, wherein the acid gas comprises carbon dioxide, sulfur dioxide, hydrogen sulfide, or a combination thereof.

Example 43: The method of any examples herein, particularly examples 37-42, wherein the acid gas comprises CO₂, hydrogen sulfide, or a combination thereof.

Example 44: The method of any examples herein, particularly examples 37-43, wherein the acid gas comprises CO₂.

Example 45: The method of any examples herein, particularly examples 37-44, wherein the fluid stream comprises an industrial gas stream such as a power plant emission (e.g., flue gas), ambient air, or a combination thereof.

Example 46: The method of any examples herein, particularly examples 37-45, wherein the fluid stream comprises ambient air.

Example 47: The method of any examples herein, particularly examples 37-46, wherein the fluid stream comprises ambient air and the acid gas comprises carbon dioxide (e.g., wherein the method comprises direct air capture).

Example 48: The method of any examples herein, particularly examples 37-45, wherein the fluid stream comprises natural gas, byproducts of a chemical reaction, post-combustion flue gas, or a combination thereof.

Example 49: A method of making the compound of any examples herein, particularly examples 1-33.

Example 50: The method of any examples herein, particularly example 49, wherein the compound is derived at least in part from natural products (e.g., biosourced).

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

What is claimed is:

1. A compound for capturing an acid gas from a fluid stream, wherein the compound is defined by Formula I: 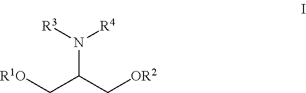
wherein
R¹ and R² are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy; and
R³ and R⁴ are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy, or wherein, as valence permits, R³ and R⁴, together with the atoms to which they are attached, form a 3-10 membered substituted or unsubstituted cyclic moiety, optionally including 1 to 3 additional heteroatoms;
with the proviso that at least one of R¹-R⁴ is not H.

2. The compound of claim 1, wherein:
R¹ and R² are each independently substituted or unsubstituted C₁₋₂₀ alkyl, or substituted or unsubstituted C₁-C₂₀ alkoxy; and
R³ and R⁴ are each independently H, substituted or unsubstituted C₁₋₂₀ alkyl, or substituted or unsubstituted C₁-C₂₀ alkoxy, or wherein, as valence permits, R³ and R⁴, together with the atoms to which they are attached, form a 3-10 membered substituted or unsubstituted cyclic moiety, optionally including 1 to 3 additional heteroatoms.

3. The compound of claim 1, wherein R³ and R⁴ are the same.

4. The compound of claim 1, wherein R³ and R⁴ are different.

5. The compound of claim 1, wherein R³ and R⁴ are each independently H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃-C₂₀ aryl, substituted or unsubstituted C₄-C₂₀ alkylaryl, substituted or unsubstituted C₂-C₂₀ cycloalkyl, substituted or unsubstituted C₁-C₂₀ acyl, substituted or unsubstituted C₁-C₂₀ alkoxy, or substituted or unsubstituted C₃-C₂₀ aryloxy, or wherein, as valence permits, R³ and R⁴, together with the atoms to which they are attached, form a 3-10 membered substituted or unsubstituted cyclic moiety, optionally including 1 to 3 additional heteroatoms.

6. The compound of claim 1, wherein R³ and R⁴ are each independently H, substituted or unsubstituted C₁₋₂₀ alkyl, or substituted or unsubstituted C₁-C₂₀ alkoxy, or wherein, as valence permits, R³ and R⁴, together with the atoms to which they are attached, form a 3-10 membered substituted or unsubstituted cyclic moiety, optionally including 1 to 3 additional heteroatoms.

7. The compound of claim 1, wherein R³ and R⁴ are each independently H, substituted or unsubstituted C₁₋₆ alkyl, or substituted or unsubstituted C₁-C₆ alkoxy, or wherein, as valence permits, R³ and R⁴, together with the atoms to which they are attached, form a 6 membered substituted or unsubstituted cyclic moiety, optionally including 1 additional heteroatom.

8. The compound of claim 1, wherein R³ and R⁴ are each independently H, methyl, ethyl, isopropyl, or —CH₂CH₂OCH₃, or R³ and R⁴, together with the atoms to which they are attached, form a piperazine.

9. The compound of claim 1, wherein R¹ and R² are the same.

10. The compound of claim 1, wherein R¹ and R² are different.

11. The compound of claim 1, wherein R¹ and R² are each independently substituted or unsubstituted C₁₋₂₀ alkyl, or substituted or unsubstituted C₁-C₂₀ alkoxy.

12. The compound of claim 1, wherein R¹ and R² are each independently substituted or unsubstituted C₁₋₆ alkyl, or substituted or unsubstituted C₁-C₆ alkoxy.

13. The compound of claim 1, wherein R¹ and R² are each independently C₁₋₆ alkyl optionally substituted with one or more substituents selected from the group consisting of ether, C₁-C₅ alkyl, halogen, amine, and C₁-C₅ alkoxy.

14. The compound of claim 1, wherein R¹ and R² are each independently C₁₋₆ alkyl optionally substituted with halogen.

15. The compound of claim 1, wherein R¹ and R² are each independently methyl, ethyl, isopropyl, —CH₂CF₃, —CH₂CH₂OCH₃.

16. The compound of claim 1, wherein the compound is selected from the group consisting of: 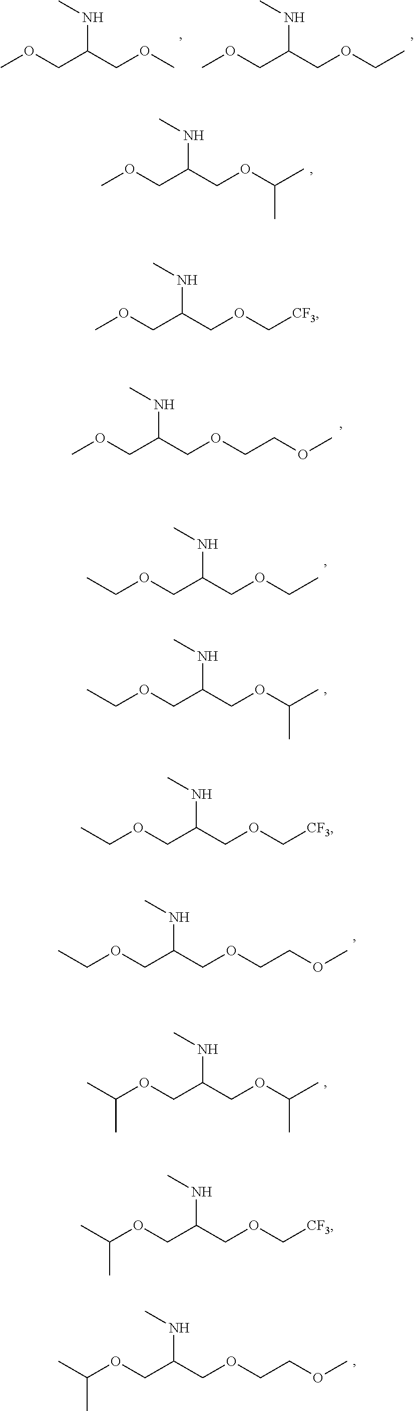 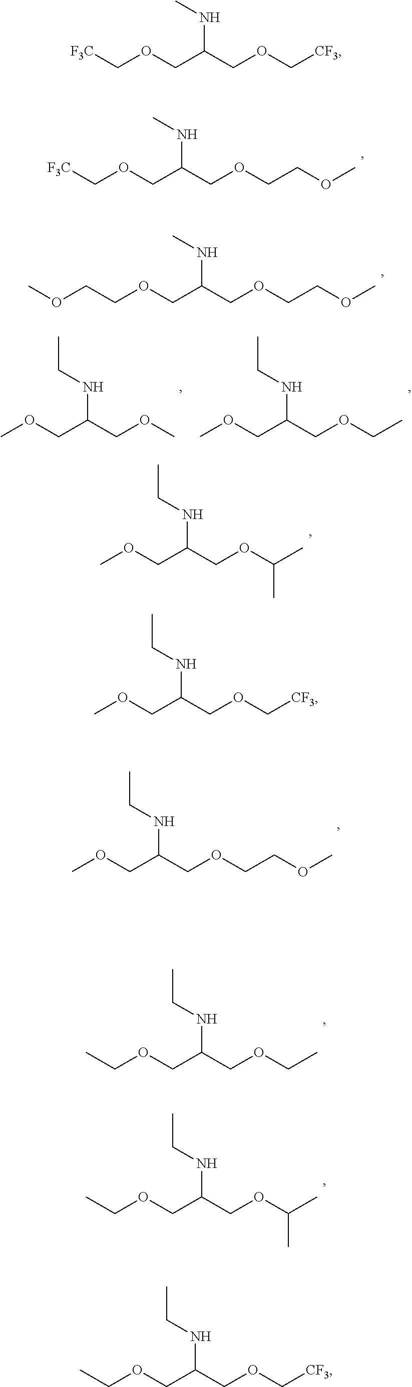 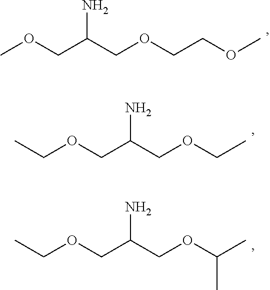 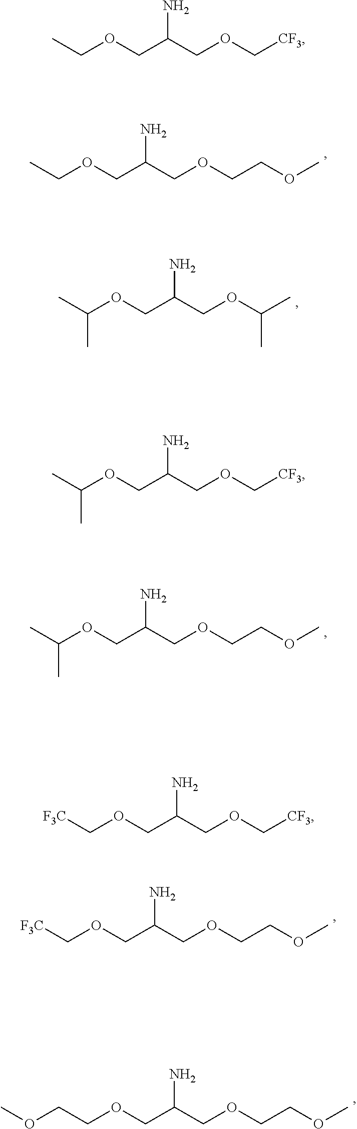 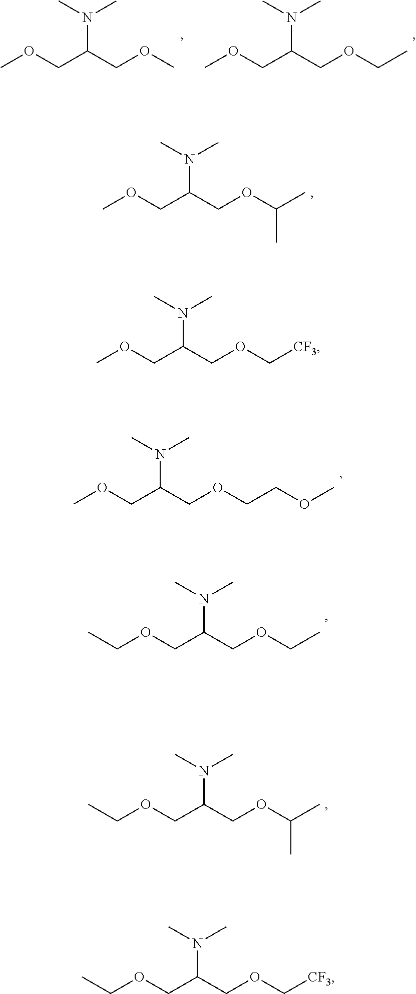 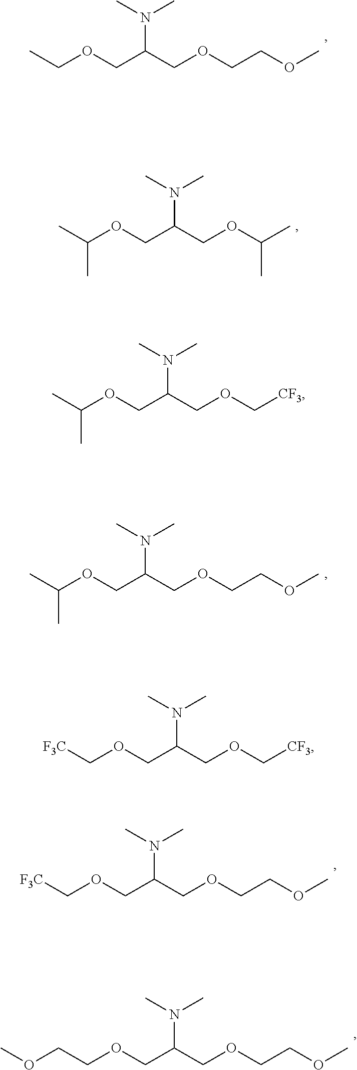 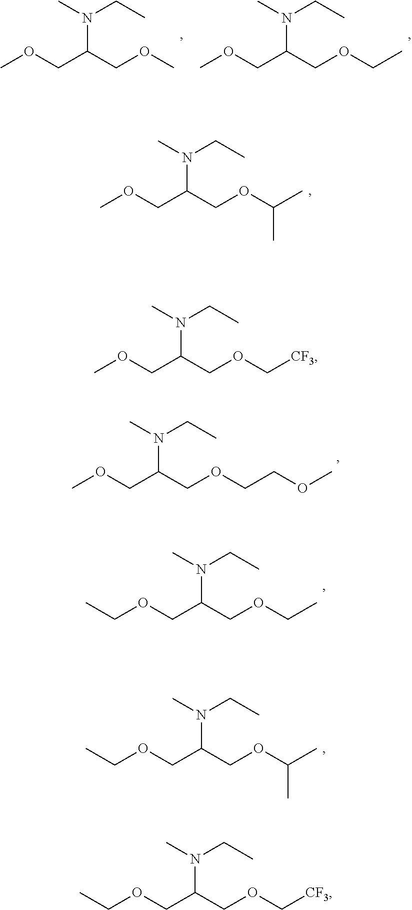 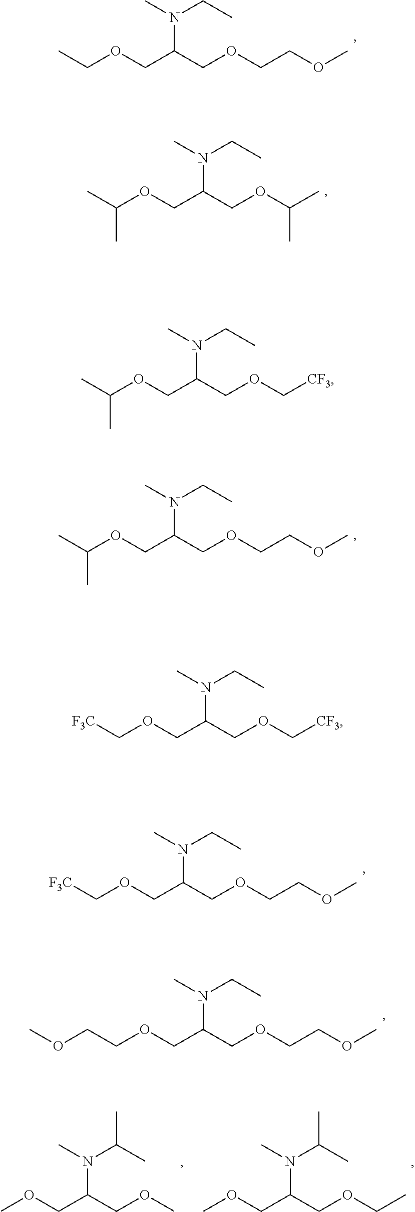 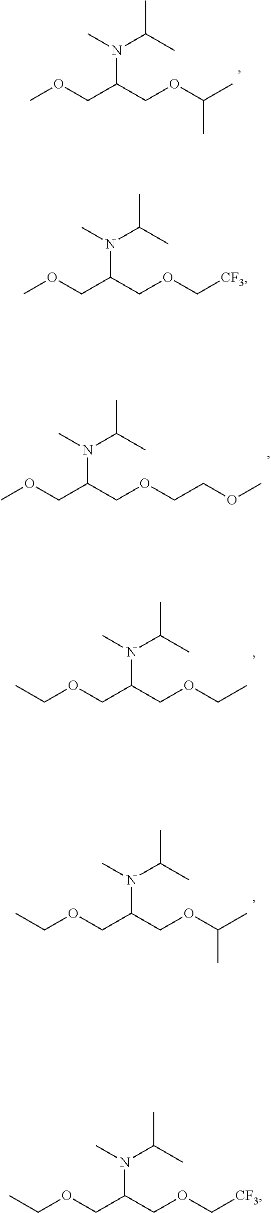 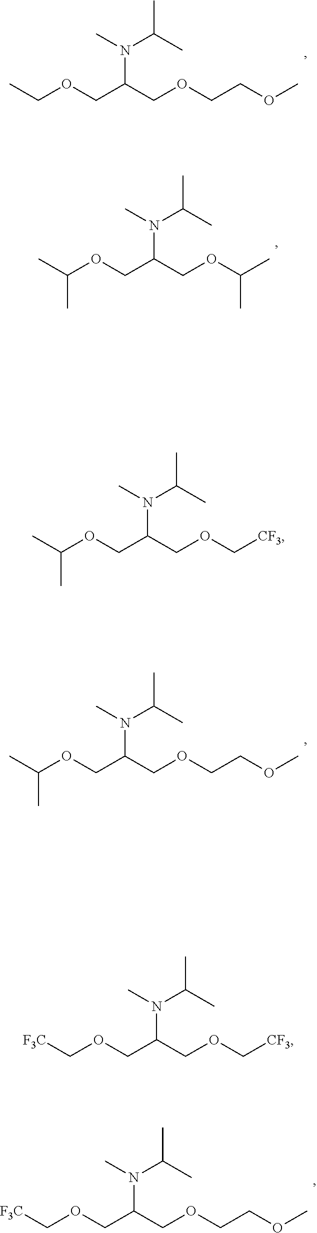 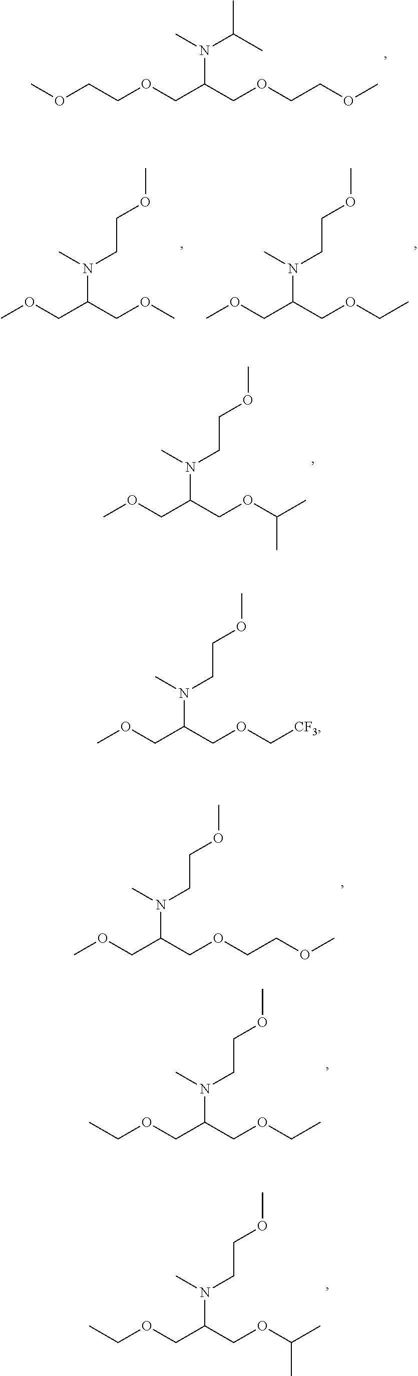 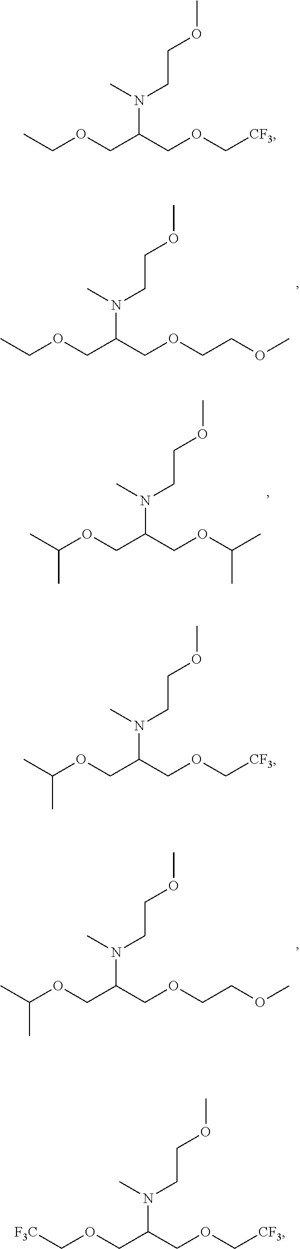 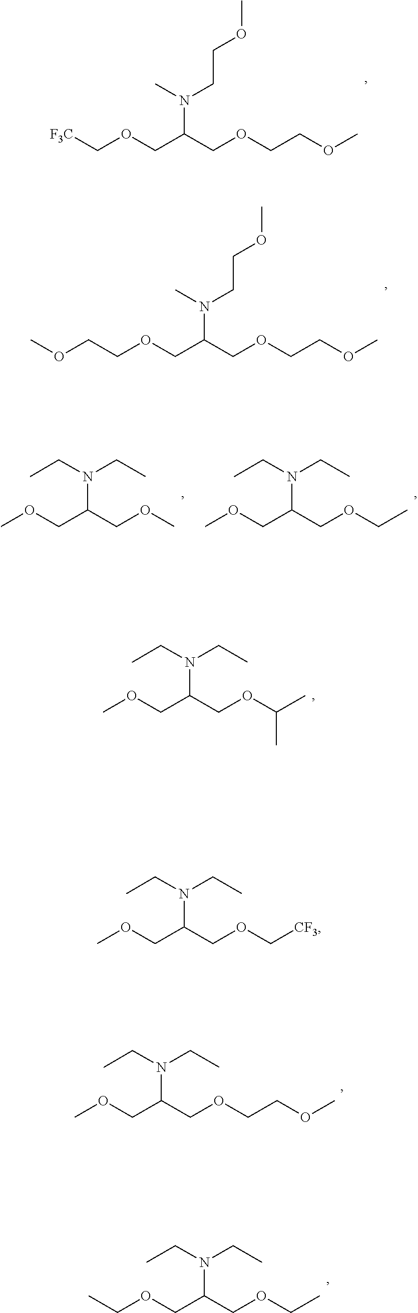 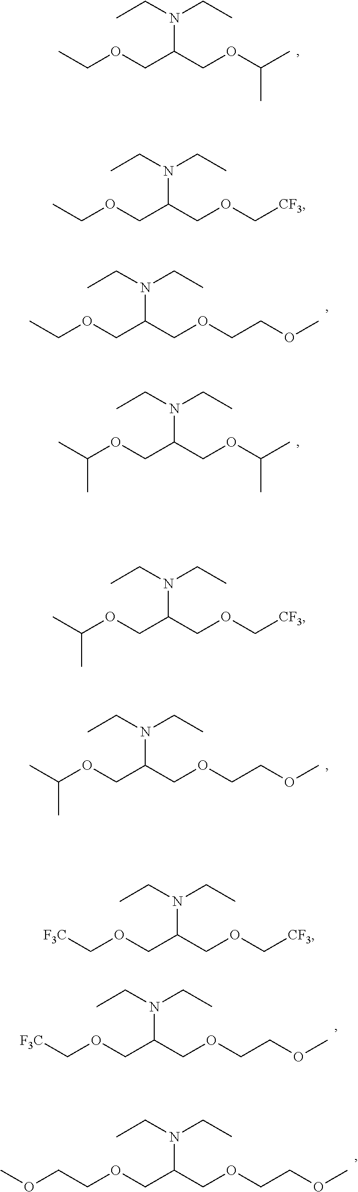 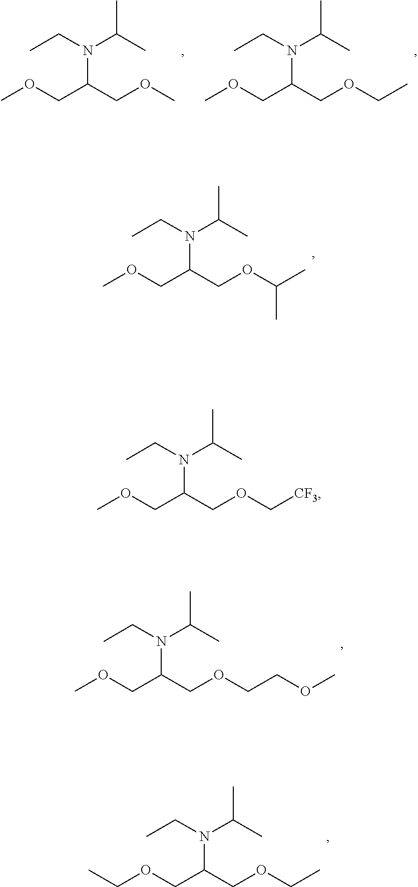 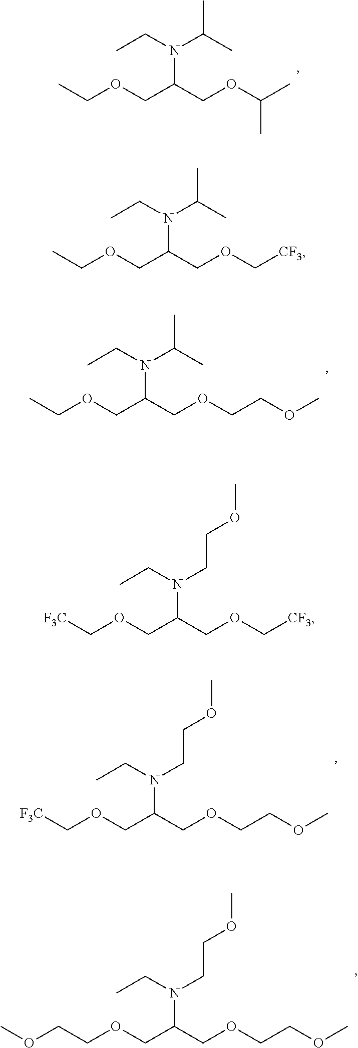 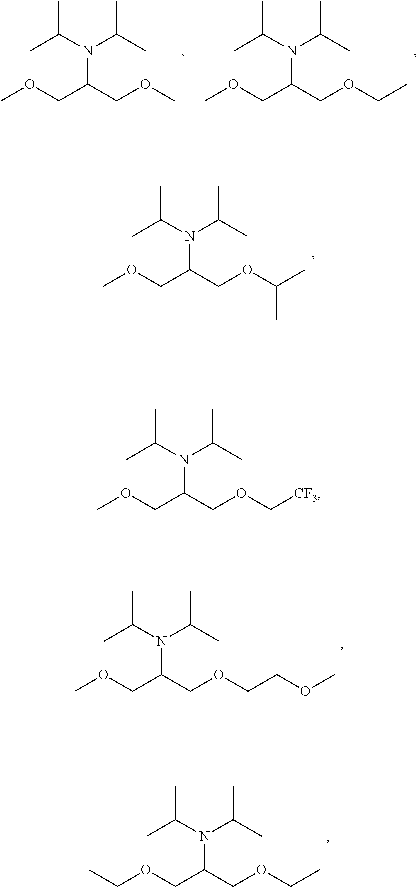 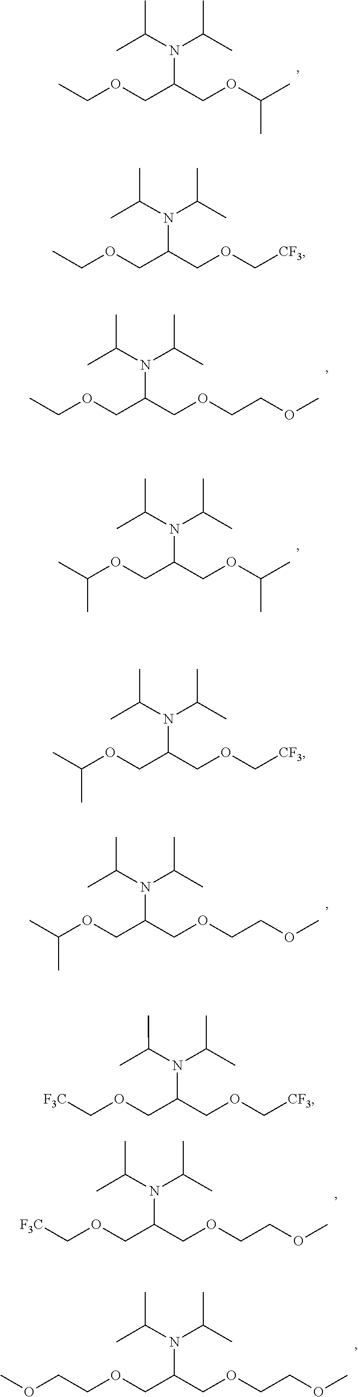 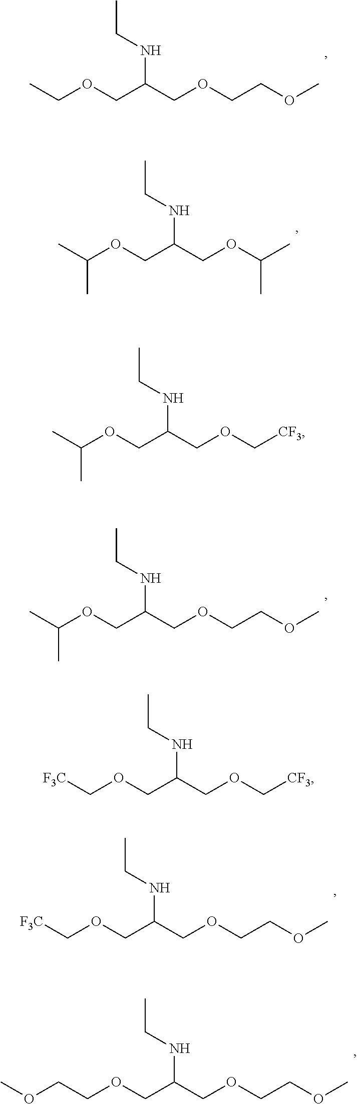 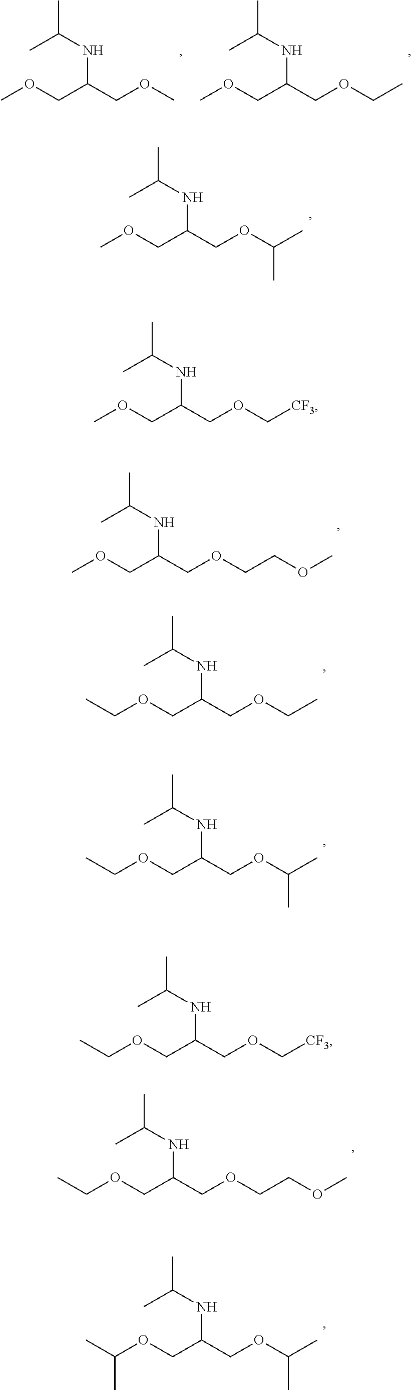 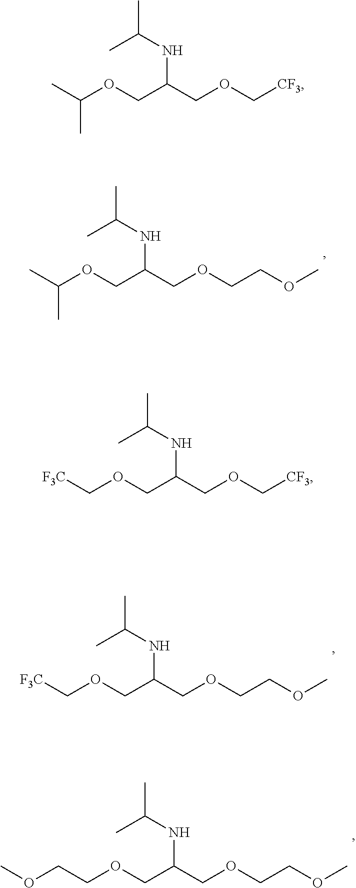 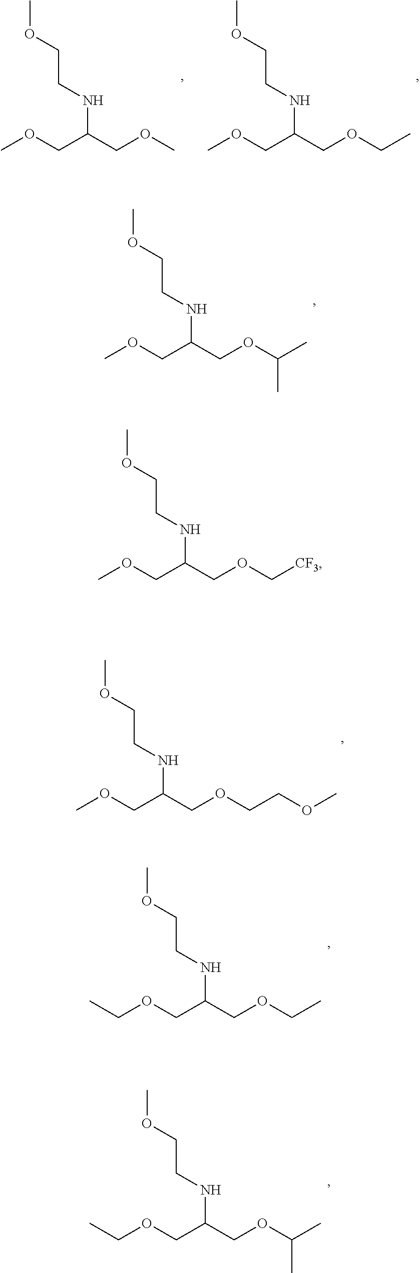 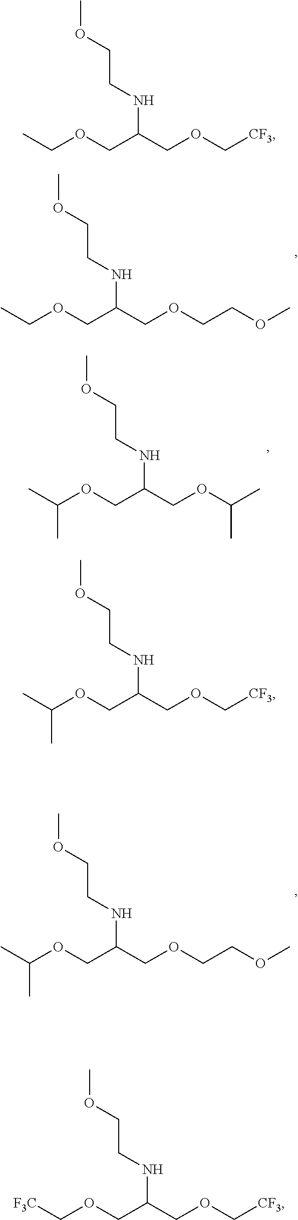 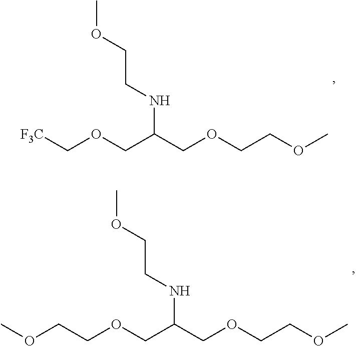 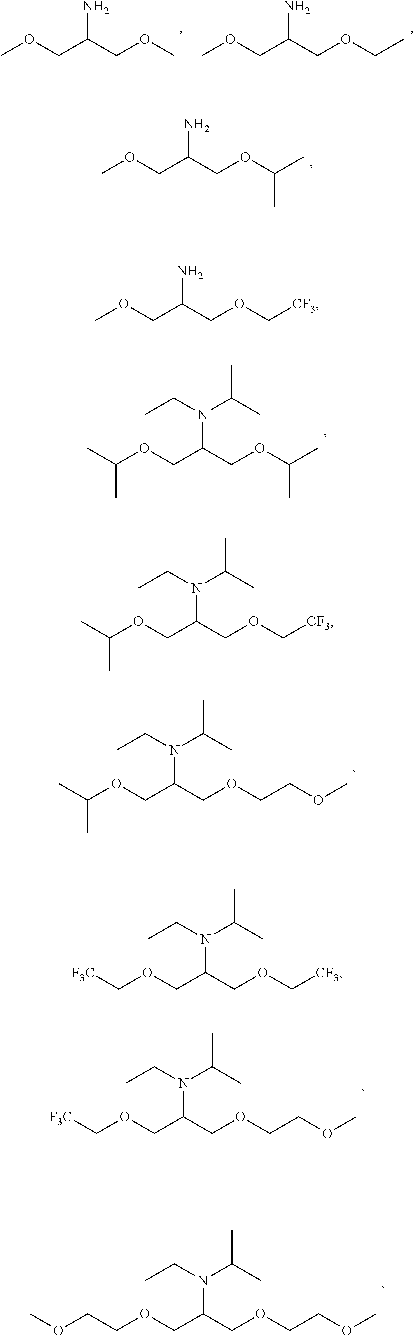 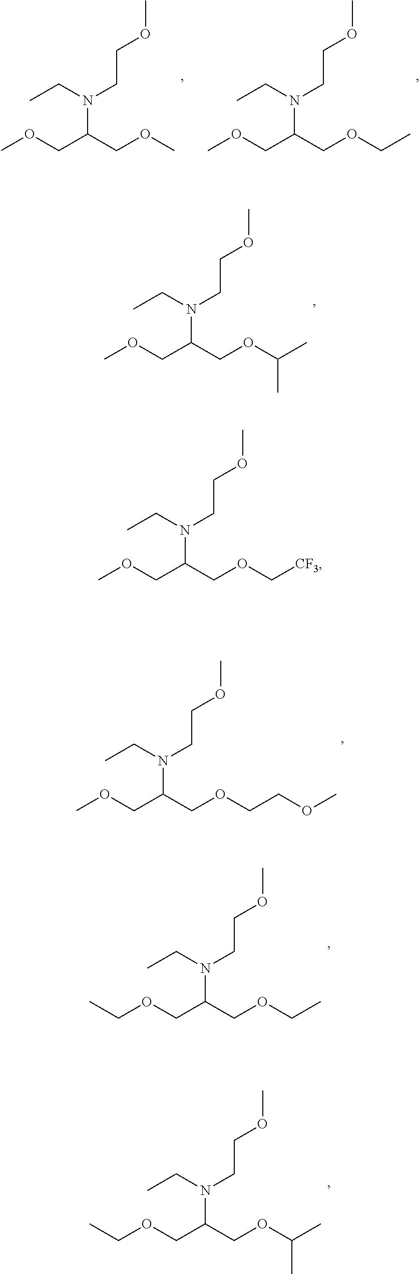 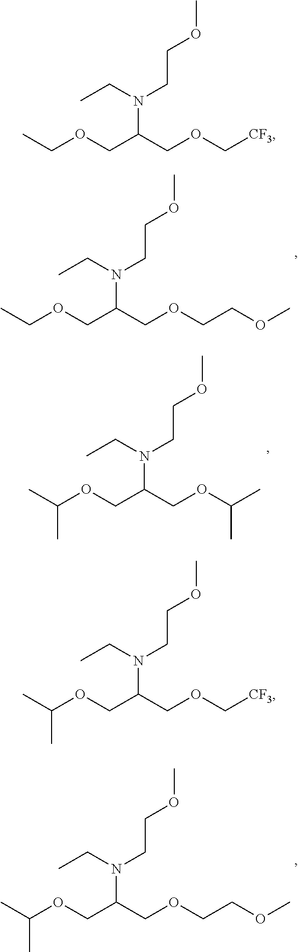 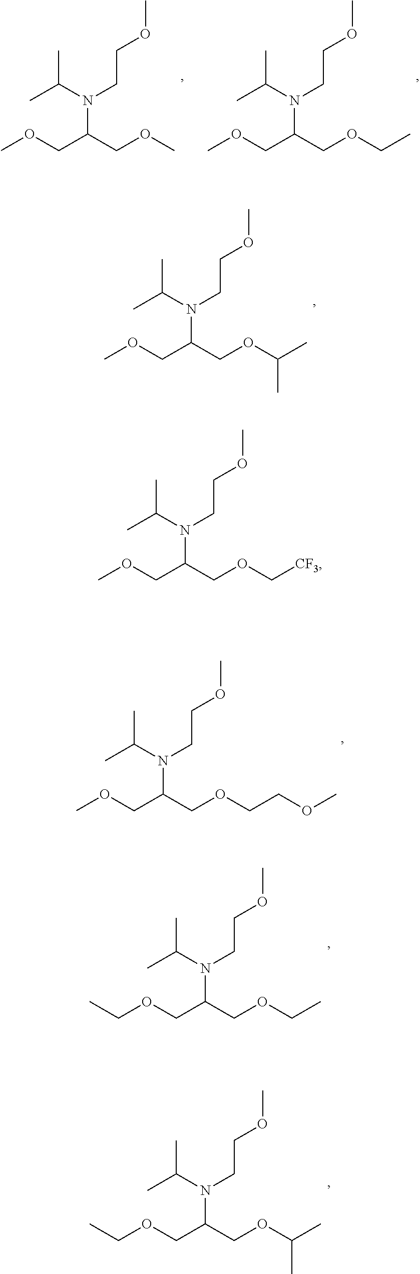 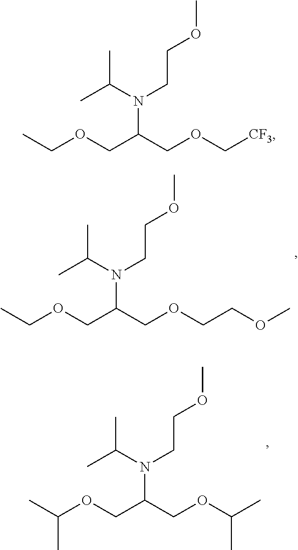 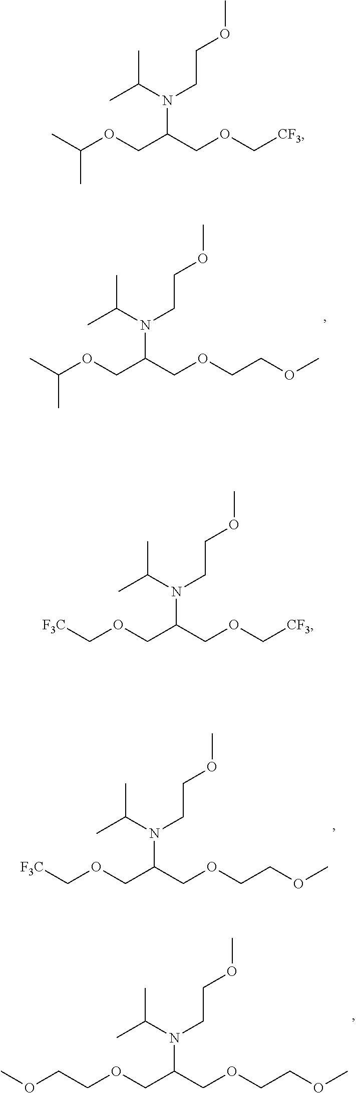 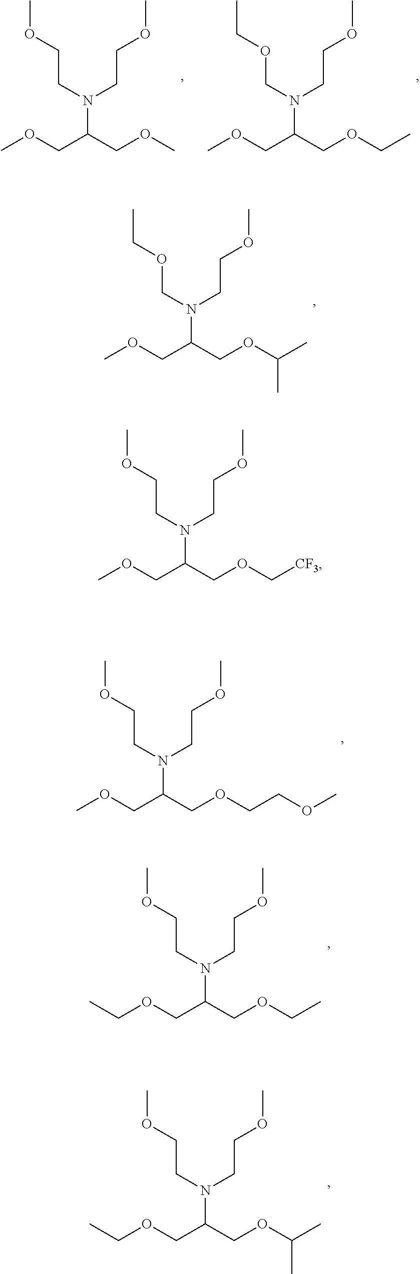 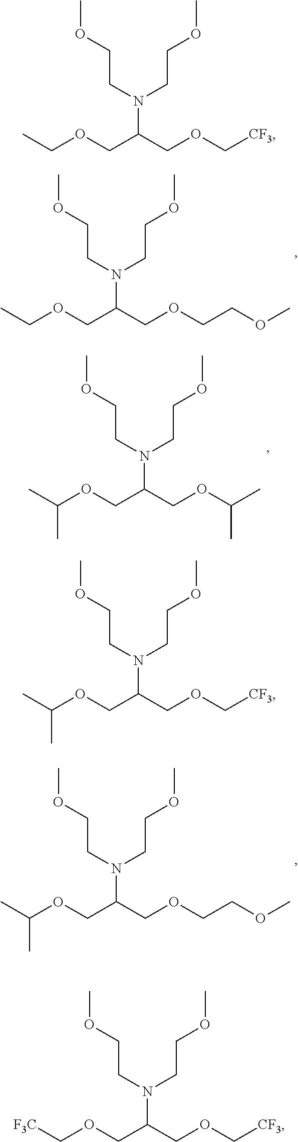 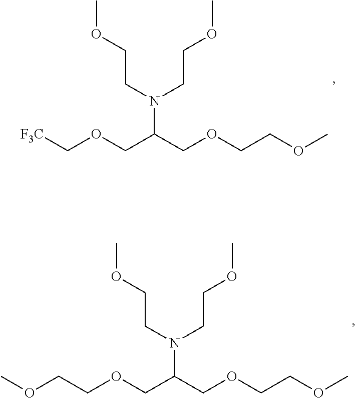 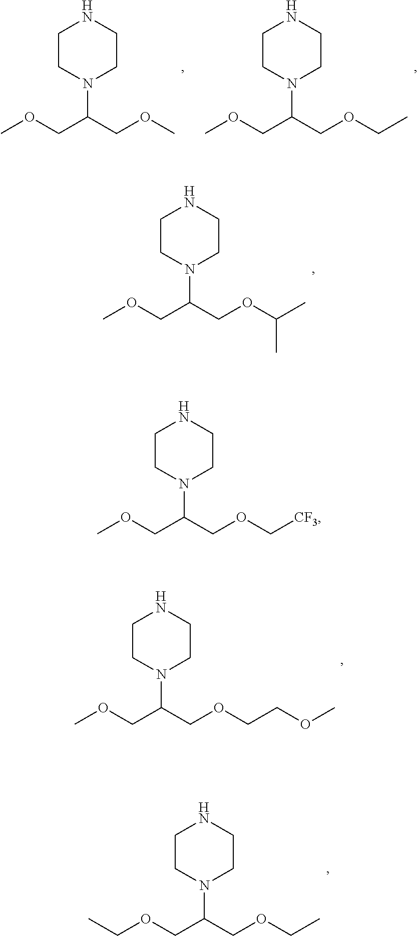 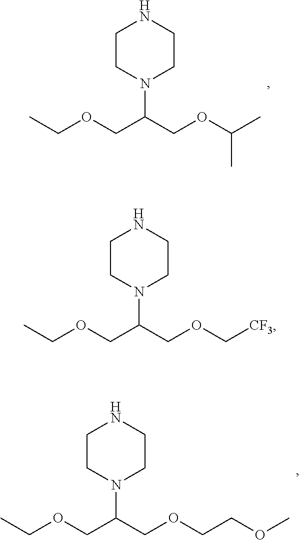 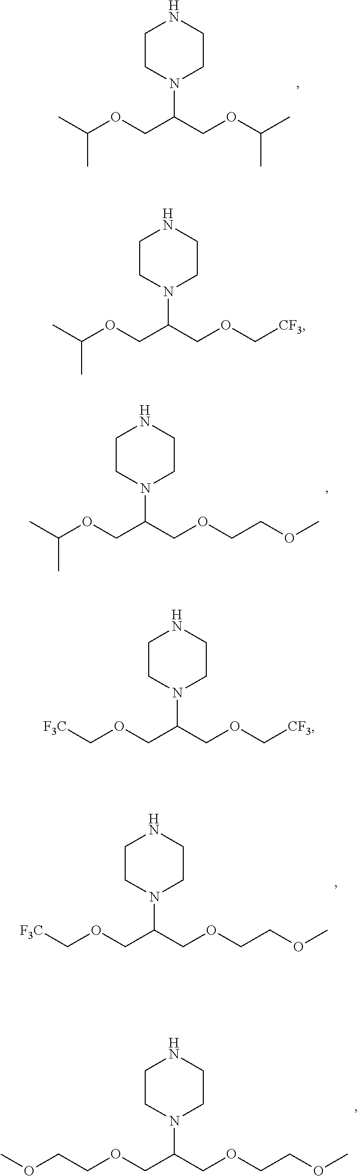
and combinations thereof.

17. The compound of claim 1, wherein the compound is selected from the group consisting of: 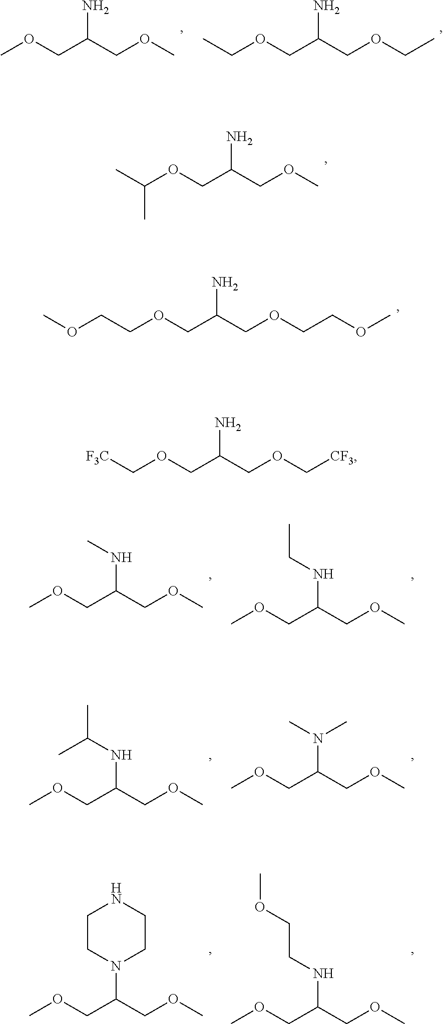
and combinations thereof.

18. A solvent system for capturing an acid gas from a fluid stream, wherein the solvent system comprises the compound of claim 1 and an additional solvent.

19. A method of use of the compound of claim 1 for capturing an acid gas from a fluid steam, the method comprising contacting the compound with the fluid stream, wherein the fluid stream comprises the acid gas, thereby capturing the acid gas from the fluid stream.

20. A method of making the compound of claim 1, wherein the compound is derived at least in part from natural products.
